(19) United States
(12) Patent Application Publication
(10) Pub. No.: US 2025/0179182 A1
(43) Pub. Date: Jun. 5, 2025

(54) METHODS OF TREATING CANCER WITH A COMBINATION OF AN ANTI-PD-1 ANTIBODY AND AN ANTI-CD30 ANTIBODY DRUG CONJUGATE

(71) Applicant: SEAGEN INC., Bothell, WA (US)

(72) Inventors: Ryan Heiser, Lake Stevens, WA (US); Scott Knowles, Seattle, WA (US); Markus Puhlmann, Seattle, WA (US)

(73) Assignee: SEAGEN INC., Bothell, WA (US)

(21) Appl. No.: 18/703,532

(22) PCT Filed: Oct. 27, 2022
(86) PCT No.: PCT/US2022/078767

§ 371 Date: Apr. 22, 2024

Related U.S. Application Data
(60) Provisional application No. 63/273,411, filed on Oct. 29, 2021.

(51) Int. Cl. C07K 16/28; A61K 39/00; A61K 47/68; A61P 35/04

(52) U.S. Cl. CPC C07K 16/2818; A61K 47/68031; A61K 47/6849; A61K 47/6851; A61P 35/04; C07K 16/2878; A61K 2039/507; A61K 2039/545

(57) ABSTRACT

The invention provides an anti-PD-1 antibody and an antibody-drug conjugate that binds to CD30 and their use in methods of treating solid tumors. The invention also provides compositions and kits comprising the anti-PD-1 antibody and the antibody-drug conjugate that binds to CD30 for use in treating solid tumors.

### CROSS REFERENCE TO RELATED APPLICATION

This application claims the benefit of U.S. Provisional Application No. 63/273,411, filed Oct. 29, 2021, which is incorporated by reference in its entirety for all purposes.

### REFERENCE TO A SEQUENCE LISTING

This application includes an electronic sequence listing in a file name 0030-02011PC_Sequence_Listing, created on Oct. 4, 2022 and containing 32 Kb, which is hereby incorporated by reference.

### TECHNICAL FIELD

The present invention relates to methods of treating solid tumors with a combination of an anti-PD-1 antibody and an anti-CD30 antibody-drug conjugate.

### BACKGROUND

CD30 is a 120 kilodalton membrane glycoprotein (Froese et al., 1987, J. Immunol. 139:2081-87) and a member of the TNF-receptor superfamily. CD30 is a proven marker of malignant cells in Hodgkin lymphoma and anaplastic large cell lymphoma (ALCL). CD30 was originally identified on cultured Hodgkin's-Reed Steinberg (H-RS) cells using the monoclonal antibody Ki-1 (Schwab et al., 1982, Nature 299:65-67).

CD30 has limited expression on normal tissues in humans. This makes CD30 an attractive target for cancer therapies. CD30 expression has been identified, however, on only a small number of cancers.

Human cancers harbor numerous genetic and epigenetic alterations, generating neoantigens potentially recognizable by the immune system (Sjoblom et al., 2006, Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities. Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of an antibody, ipilimumab (YERVOY®), that binds to and inhibits CTLA-4 for the treatment of patients with advanced melanoma (Hodi et al., 2010, N Engl J Med 363:711-23) and the development of an antibody, pembrolizumab (formerly lambrolizumab; USAN Council Statement, 2013), that binds specifically to the Programmed Death-1 (PD-1) receptor and block the inhibitory PD-1/PD-1 ligand pathway (Hamid and Carvajal, Expert Opin Biol Ther 13 (6): 847-61 (2013); and McDermott and Atkins, Cancer Med 2 (5): 662-73 (2013)).

A significant proportion of patients who initially demonstrate a clinical response to checkpoint inhibitors (CPIs), such as pembrolizumab, acquire resistance over time. The development of resistance is presumed to be due to compensatory mechanisms within the tumor microenvironment (TME) that develop in order to evade the antitumor effects of immunotherapy.

T regulatory cells (Tregs) are essential modulators of T cell immune responses, limiting chronic inflammation and protecting normal tissues from autoimmunity. T regulatory cells are also implicated in maintaining immune-suppressive conditions in the tumor microenvironment, abrogating cytotoxic anti-tumor immunosurveillance. Analysis of clinical tumor samples has shown increased densities of intratumoral Tregs associated with poor clinical outcomes in a number of cancer types (Fridman, 2012, Nature Reviews Cancer; Charoentong, 2017, Cell Reports 18:248-262). Recent transcriptomic analyses of intratumoral Tregs isolated from breast, lung, and colorectal cancer tissues showed TNFSFR8 (CD30) to be among transcripts differentially upregulated compared to Tregs isolated from adjacent normal tissue and circulating in blood (Plitas, 2016, Immunity, 45:1122-1134; De Simone, 2016, Immunity, 45:1135-1147). The functional significance of heightened CD30 transcript expression in Tregs remains unclear. Given the protective role of Tregs in promoting immune homeostasis in normal tissues, there is considerable interest in developing cancer therapeutics that preferentially target intratumoral Tregs, while sparing those in non-diseased tissues. Therefore, there appears to be a need for therapies that can selectively control the activity of immune cells that are involved in pathogenesis of cancer, such as the activity of T regulatory cells.

Targeted therapy of multiple non-redundant molecular pathways regulating immune responses can enhance antitumor immunotherapy. However, not all combinations have acceptable safety and/or efficacy. There remains a need for combination therapies with an acceptable safety profile and high efficacy that enhance antitumor immune responses compared to monotherapy and other immunotherapy combinations.

All references cited herein, including patent applications, patent publications, and scientific literature, are herein incorporated by reference in their entirety, as if each individual reference were specifically and individually indicated to be incorporated by reference.

### SUMMARY

Provided herein is a method of treating cancer in a subject, the method comprising administering to the subject an anti-PD-1 antibody or an antigen-binding fragment thereof and an anti-CD30 antibody-drug conjugate, wherein the cancer is a solid tumor selected from the group consisting of non-small cell lung cancer (NSCLC), melanoma, head and neck cancer, renal cell carcinoma, microsatellite instability-high or mismatch repair deficient cancer, bladder cancer, colon cancer, rectal cancer, gastric cancer, gastroesophageal junction carcinoma, esophageal cancer, cervical cancer, hepatocellular carcinoma, endometrial carcinoma, Merkel cell carcinoma, cutaneous squamous cell carcinoma, breast cancer, and tumor mutational burden-high cancer, wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and
   wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22. In some embodiments, the anti-PD-1 antibody or antigen-binding fragment thereof inhibits the activity of PD-1. In some embodiments, the anti-PD-1 antibody or antigen-binding fragment thereof comprises a heavy chain variable region comprising the amino acid sequence of SEQ ID NO: 31 and a light chain variable region comprising the amino acid sequence of SEQ ID NO: 32. In some embodiments, the anti-PD-1 antibody or antigen-binding fragment thereof is pembrolizumab or a biosimilar thereof. In some embodiments, the anti-PD-1 antibody or antigen-binding fragment thereof is pembrolizumab. In some embodiments, the anti-CD30 antibody-drug conjugate comprises an anti-CD30 antibody or antigen-binding fragment thereof conjugated to a therapeutic agent. In some embodiments, the anti-CD30 antibody or antigen-binding fragment thereof of the anti-CD30 antibody drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
   wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6. In some embodiments, the anti-CD30 antibody or antigen-binding fragment thereof of the anti-CD30 antibody drug conjugate comprises a heavy chain variable region comprising the amino acid sequence of SEQ ID NO:7 and a light chain variable region comprising the amino acid sequence of SEQ ID NO:8. In some embodiments, the anti-CD30 antibody or antigen-binding fragment thereof of the anti-CD30 antibody drug conjugate is cAC10 or a biosimilar thereof. In some embodiments, the anti-CD30 antibody or antigen-binding fragment thereof of the anti-CD30 antibody drug conjugate is cAC10. In some embodiments, the anti-CD30 antibody-drug conjugate further comprise a linker between the anti-CD30 antibody or antigen-binding fragment thereof and the therapeutic agent. In some embodiments, the linker is a cleavable peptide linker. In some embodiments, the cleavable peptide linker has a formula: -MC-vc-PAB-, wherein:
a) MC is:

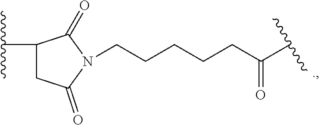 [SMILES: *C(=O)CCCCCN1C(=O)CC(*)C1=O]

b) vc is the dipeptide valine-citrulline, and
c) PAB is:

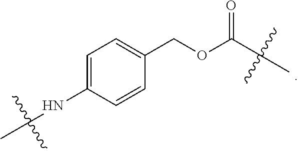 [SMILES: *Nc1ccc(COC(*)=O)cc1]

In some embodiments, the therapeutic agent is an auristatin. In some embodiments, the auristatin is monomethyl auristatin E. In some embodiments, the anti-CD30 antibody-drug conjugate is brentuximab vedotin or a biosimilar thereof. In some embodiments, the anti-CD30 antibody-drug conjugate is brentuximab vedotin. In some embodiments, the anti-PD-1 antibody or antigen-binding fragment thereof is administered at a flat dose ranging from about 50 mg to about 500 mg. In some embodiments, the anti-PD-1 antibody or antigen-binding fragment thereof is administered at a flat dose of about 200 mg. In some embodiments, the anti-PD-1 antibody or antigen-binding fragment thereof is administered at a flat dose of 200 mg. In some embodiments, the anti-PD-1 antibody or antigen-binding fragment thereof is administered at a flat dose of about 400 mg. In some embodiments, the anti-PD-1 antibody or antigen-binding fragment thereof is administered at a flat dose of 400 mg. In some embodiments, the anti-PD-1 antibody or antigen-binding fragment thereof is administered once about every 1 week, once about every 2 weeks, once about every 3 weeks, once about every 4 weeks, once about every 5 weeks, or once about every 6 weeks. In some embodiments, the anti-PD-1 antibody or antigen-binding fragment thereof is administered once about every 3 weeks. In some embodiments, the anti-PD-1 antibody or antigen-binding fragment thereof is administered once every 3 weeks. In some embodiments, the anti-PD-1 antibody or antigen-binding fragment thereof is administered once about every 6 weeks. In some embodiments, the anti-PD-1 antibody or antigen-binding fragment thereof is administered once every 6 weeks. In some embodiments, the anti-CD30 antibody-drug conjugate is administered at a dose of about 0.6 mg/kg to about 2.3 mg/kg of the subject's body weight. In some embodiments, the anti-CD30 antibody-drug conjugate is administered at a dose of about 1.8 mg/kg of the subject's body weight. In some embodiments, the anti-CD30 antibody-drug conjugate is administered at a dose of 1.8 mg/kg of the subject's body weight. In some embodiments, the anti-CD30 antibody-drug conjugate is administered once about every 1 week, once about every 2 weeks, once about every 3 weeks, once about every 4 weeks, once about every 5 weeks, or once about every 6 weeks. In some embodiments, the anti-CD30 antibody-drug conjugate is administered once about every 3 weeks. In some embodiments, the anti-CD30 antibody-drug conjugate is administered once every 3 weeks. In some embodiments, the subject has not been previously treated for the cancer. In some embodiments, the subject has been previously treated for the cancer and the subject failed treatment, did not respond to treatment, progressed on treatment, or relapsed after first-line treatment. In some embodiments, the subject failed treatment, did not respond to treatment, progressed on treatment, or relapsed after treatment with an anti-PD-1 monoclonal antibody. In some embodiments, the subject is currently on PD-1 checkpoint inhibitor therapy. In some embodiments, the subject was on a PD-1 checkpoint inhibitor therapy as the last previous line of therapy within 90 days of being administered the combination of the anti-PD-1 antibody or an antigen-binding fragment thereof and the anti-CD30 antibody-drug conjugate. In some embodiments, the subject was previously treated for the cancer with surgery. In some embodiments, the subject was treated with a PD-1 checkpoint inhibitor adjuvant therapy after surgery. In some embodiments, the cancer is metastatic. In some embodiments, the cancer is NSCLC. In some embodiments, the cancer is squamous NSCLC. In some embodiments, the cancer is nonsquamous NSCLC. In some embodiments, the cancer is primary refractory NSCLC. In some embodiments, the subject progressed without a prior objective response or has stable disease for less than 6 months. In some embodiments, the cancer is relapsed NSCLC. In some embodiments, the subject progressed after having developed a prior objective response of complete response or partial response (CR/PR) for at least 3 months or stable disease for at least 6 months. In some embodiments, the subject does not have a known targetable EGFR, ALK, ROS1, or BRAF mutation. In some embodiments, the cancer is melanoma. In some embodiments, the cancer is cutaneous melanoma. In some embodiments, the cancer is primary refractory melanoma. In some embodiments, the subject progressed without a prior objective response or has stable disease for less than 6 months. In some embodiments, the cancer is relapsed melanoma. In some embodiments, the subject progressed after having developed a prior objective response of complete response or partial response (CR/PR) for at least 3 months or stable disease for at least 6 months. In some embodiments, the subject does not have a targetable gene mutation. In some embodiments, the subject is a BRAF-V660E/V600K subject who failed targeted therapy. In some embodiments, the cancer is head and neck cancer. In some embodiments, the head and neck cancer is squamous cell carcinoma. In some embodiments, the cancer is renal cell carcinoma. In some embodiments, the cancer is microsatellite instability-high or mismatch repair deficient cancer. In some embodiments, the cancer is microsatellite instability-high or mismatch repair deficient colorectal cancer. In some embodiments, the cancer is bladder cancer. In some embodiments, the bladder cancer is urothelial cancer. In some embodiments, the cancer is high risk, non-muscle invasive bladder cancer. In some embodiments, the cancer is colon cancer. In some embodiments, the cancer is rectal cancer. In some embodiments, the cancer is gastric cancer. In some embodiments, the cancer is gastroesophageal junction carcinoma. In some embodiments, the cancer is gastroesophageal junction adenocarcinoma. In some embodiments, the cancer is esophageal cancer. In some embodiments, the cancer is cervical cancer. In some embodiments, the cancer is hepatocellular carcinoma. In some embodiments, the cancer is endometrial carcinoma. In some embodiments, the cancer is Merkel cell carcinoma. In some embodiments, the cancer is cutaneous squamous cell carcinoma. In some embodiments, the cancer is breast cancer. In some embodiments, the breast cancer is triple-negative breast cancer. In some embodiments, the cancer is tumor mutational burden-high cancer. In some embodiments, the subject has not been previously treated with the anti-CD30 antibody-drug conjugate. In some embodiments, the subject does not have known active central nervous system metastases and/or carcinomatous meningitis. In some embodiments, the route of administration for the anti-PD-1 antibody or antigen-binding fragment thereof is intravenous or subcutaneous. In some embodiments, the route of administration for the anti-PD-1 antibody or antigen-binding fragment thereof is intravenous. In some embodiments, the route of administration for the anti-PD-1 antibody or antigen-binding fragment thereof is subcutaneous. In some embodiments, the route of administration for the anti-CD30 antibody-drug conjugate is intravenous. In some embodiments, the anti-PD-1 antibody or antigen-binding fragment thereof and the anti-CD30 antibody-drug conjugate are administered sequentially. In some embodiments, the anti-PD-1 antibody or antigen-binding fragment thereof and the anti-CD30 antibody-drug conjugate are administered simultaneously. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1. In some embodiments, the subject has a tumor that expresses PD-L1 (TPS≥1). In some embodiments, the subject has a tumor that has high PD-L1 expression (TPS≥50). In some embodiments, the subject has a tumor that expresses PD-L1 (CPS≥1). In some embodiments, a tumor derived from the cancer comprises one or more cells that express PD-L1, PD-L2, or both PD-L1 and PD-L2. In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T cells from the subject express PD-1. In some embodiments, less than about 0.1%, less than about 0.5%, less than about 1%, less than about 2%, less than about 3%, less than about 4%, or less than about 5% of cancer cells from the subject express CD30. In some embodiments, the cancer cells from the subject do not express CD30. In some embodiments, the cancer cells from the subject have been determined to not express CD30. In some embodiments, the cancer cells from the subject have been assessed for CD30 expression. In some embodiments, the cancer cells from the subject have not been assessed for CD30 expression. In some embodiments, the cancer cells from the subject have been screened for CD30 expression and the cancer cells have been determined to not express CD30. In some embodiments, the cancer cells from the subject have not been screened for CD30 expression. In some embodiments, the cancer cells from the subject have been screened for CD30 expression and less than about 0.1%, less than about 0.5%, less than about 1%, less than about 2%, less than about 3%, less than about 4%, or less than about 5% of the cancer cells have been determined to express CD30. In some embodiments, one or more therapeutic effects in the subject is improved after administration of the anti-CD30 antibody-drug conjugate and the anti-PD-1 antibody or antigen-binding fragment thereof relative to a baseline. In some embodiments, the one or more therapeutic effects is selected from the group consisting of: size of a tumor derived from the cancer, objective response rate, duration of response, time to response, progression free survival, and overall survival. In some embodiments, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-CD30 antibody-drug conjugate and the anti-PD-1 antibody or antigen-binding fragment thereof. In some embodiments, the objective response rate is at least about 20%, at least about 25%, at least about 30%, at least about 35%, at least about 40%, at least about 45%, at least about 50%, at least about 60%, at least about 70%, or at least about 80%. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate and the anti-PD-1 antibody or antigen-binding fragment thereof. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate and the anti-PD-1 antibody or antigen-binding fragment thereof. In some embodiments, the duration of response to the antibody-drug conjugate is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate and the anti-PD-1 antibody or antigen-binding fragment thereof. In some embodiments, the subject has one or more adverse events and is further administered an additional therapeutic agent to eliminate or reduce the severity of the one or more adverse events. In some embodiments, the subject is at risk of developing one or more adverse events and is further administered an additional therapeutic agent to prevent or reduce the severity of the one or more adverse events. In some embodiments, the one or more adverse events is a grade 3 or greater adverse event. In some embodiments, the one or more adverse events is a serious adverse event. In some embodiments, administering the anti-CD30 antibody-drug conjugate decreases the number of CD30+T regulatory cells (Tregs) in the subject. In some embodiments, administration of the anti-PD-1 antibody or an antigen-binding fragment thereof results in an upregulation in the amount of CD30 expressed by Tregs by at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the amount of CD30 expressed by Tregs before administration of the anti-PD-1 antibody or an antigen-binding fragment thereof. In some embodiments, administering the anti-PD-1 antibody or an antigen-binding fragment thereof and the anti-CD30 antibody-drug conjugate decreases the number of myeloid-derived suppressor cells (MDSCs) in the subject. In some embodiments, administering the anti-PD-1 antibody or an antigen-binding fragment thereof and the anti-CD30 antibody-drug conjugate increases the proliferation of T cells in the subject. In some embodiments, administering the anti-PD-1 antibody or an antigen-binding fragment thereof and the anti-CD30 antibody-drug conjugate increases the infiltration of T cells in the cancer. In some embodiments, the subject is a human. In some embodiments, the anti-CD30 antibody-drug conjugate is in a pharmaceutical composition comprising the anti-CD30 antibody-drug conjugate and a pharmaceutical acceptable carrier. In some embodiments, the anti-PD-1 antibody or antigen-binding fragment thereof is in a pharmaceutical composition comprising the anti-PD-1 antibody or antigen-binding fragment thereof and a pharmaceutical acceptable carrier.

### BRIEF DESCRIPTION OF THE DRAWINGS

FIG. 1A-1H is a series of graphs showing the percent change from baseline in tumor measurement for subjects treated with a combination of brentuximab vedotin and pembrolizumab in various indications. FIG. 1A and FIG. 1E refractory metastatic cutaneous melanoma. FIG. 1B and FIG. 1F relapsed metastatic cutaneous melanoma. FIG. 1C and FIG. 1G refractory non-small cell lung cancer (NSCLC). FIG. 1D and FIG. 1H relapsed NSCLC.

FIG. 2 is a series of plots showing CD30 expression in various T cell populations.

FIG. 3 is a graph showing the change in tumor volume over time in huCD30+/− mice implanted with CT26 syngeneic colon carcinoma tumor cells after treatment with a control antibody-drug conjugate or an anti-CD30 antibody-drug conjugate.

FIG. 4A-4C is a series of graphs showing that CD30 expression is enriched on T regulatory cells in dissociated NSCLC tumors and is upregulated following treatment with an anti-PD-1 blocking antibody.

### DETAILED DESCRIPTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. Unless stated otherwise, the numbering of amino acid residues in the constant region is according to the EU-index as described in Kabat et al., Sequences of Proteins of Immunological Interest, 5th Ed. Public Health Service, National Institutes of Health, Bethesda, MD. (1991). An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “variable region” or “variable domain” refers to the domain of an antibody heavy or light chain that is involved in binding the antibody to antigen. The variable regions of the heavy chain and light chain (V_{H} and V_{L}, respectively) of a native antibody may be further subdivided into regions of hypervariability (or hypervariable regions, which may be hypervariable in sequence and/or form of structurally defined loops), also termed complementarity-determining regions (CDRs), interspersed with regions that are more conserved, termed framework regions (FRs). The terms “complementarity determining regions” and “CDRs,” synonymous with “hypervariable regions” or “HVRs” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each V_{H} and V_{L}, three CDRs and four FRs are typically arranged from amino-terminus to carboxy-terminus in the following order: FR1, CDR1, FR2, CDR2, FR3, CDR3, FR4 (See also Chothia and Lesk J. Mot. Biol., 195, 901-917 (1987)).

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half-life of significant periods of time, such as at least about 30 min, at least about 45 min, at least about one hour (h), at least about two hours, at least about four hours, at least about eight hours, at least about 12 hours (h), about 24 hours or more, about 48 hours or more, about three, four, five, six, seven or more days, etc., or any other relevant functionally-defined period (such as a time sufficient to induce, promote, enhance, and/or modulate a physiological response associated with antibody binding to the antigen and/or time sufficient for the antibody to recruit an effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, multispecific antibody or similar molecule.

The term “monoclonal antibody” as used herein refers to a preparation of antibody molecules that are recombinantly produced with a single primary amino acid sequence. A monoclonal antibody composition displays a single binding specificity and affinity for a particular epitope. Accordingly, the term “human monoclonal antibody” refers to antibodies displaying a single binding specificity which have variable and constant regions derived from human germline immunoglobulin sequences. The human monoclonal antibodies may be generated by a hybridoma which includes a B cell obtained from a transgenic or transchromosomal non-human animal, such as a transgenic mouse, having a genome comprising a human heavy chain transgene and a light chain transgene, fused to an immortalized cell.

An “isolated antibody” refers to an antibody that is substantially free of other antibodies having different antigenic specificities (e.g., an isolated antibody that binds specifically to CD30 or PD-1 is substantially free of antibodies that bind specifically to antigens other than CD30 or PD-1). An isolated antibody that binds specifically to CD30 or PD-1 can, however, have cross-reactivity to other antigens, such as CD30 or PD-1 molecules from different species. Moreover, an isolated antibody can be substantially free of other cellular material and/or chemicals. In one embodiment, an isolated antibody includes an antibody conjugate attached to another agent (e.g., small molecule drug). In some embodiments, an isolated anti-CD30 antibody includes a conjugate of an anti-CD30 antibody with a small molecule drug (e.g., MMAE or MMAF). In some embodiments, an isolated anti-PD-1 antibody includes a conjugate of an anti-PD-1 antibody with a small molecule drug (e.g., MMAE or MMAF).

A “human antibody” (HuMAb) refers to an antibody having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

The term “chimeric antibody” as used herein, refers to an antibody wherein the variable region is derived from a non-human species (e.g. derived from rodents) and the constant region is derived from a different species, such as human. Chimeric antibodies may be generated by antibody engineering. “Antibody engineering” is a term used generic for different kinds of modifications of antibodies, and which is a well-known process for the skilled person. In particular, a chimeric antibody may be generated by using standard DNA techniques as described in Sambrook et al., 1989, Molecular Cloning: A laboratory Manual, New York: Cold Spring Harbor Laboratory Press, Ch. 15. Thus, the chimeric antibody may be a genetically or an enzymatically engineered recombinant antibody. It is within the knowledge of the skilled person to generate a chimeric antibody, and thus, generation of the chimeric antibody according to the invention may be performed by other methods than described herein. Chimeric monoclonal antibodies for therapeutic applications are developed to reduce antibody immunogenicity. They may typically contain non-human (e.g. murine) variable regions, which are specific for the antigen of interest, and human constant antibody heavy and light chain domains. The terms “variable region” or “variable domains” as used in the context of chimeric antibodies, refers to a region which comprises the CDRs and framework regions of both the heavy and light chains of the immunoglobulin.

An “anti-antigen antibody” refers to an antibody that binds to the antigen. For example, an anti-CD30 antibody is an antibody that binds to the antigen CD30. In another example, an anti-PD-1 antibody is an antibody that binds to the antigen PD-1.

An “antigen-binding portion” or antigen-binding fragment” of an antibody refers to one or more fragments of an antibody that retain the ability to bind specifically to the antigen bound by the whole antibody. Examples of antibody fragments (e.g., antigen-binding fragment) include but are not limited to Fv, Fab, Fab′, Fab′-SH, F(ab′)₂; diabodies; linear antibodies; single-chain antibody molecules (e.g. scFv); and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y
where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % sequence identity of A to B will not equal the % sequence identity of B to A.

As used herein, the terms “binding”, “binds” or “specifically binds” in the context of the binding of an antibody to a pre-determined antigen typically is a binding with an affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less when determined by for instance BioLayer Interferometry (BLI) technology in a Octet HTX instrument using the antibody as the ligand and the antigen as the analyte, and wherein the antibody binds to the predetermined antigen with an affinity corresponding to a K_{D} that is at least ten-fold lower, such as at least 100-fold lower, for instance at least 1,000-fold lower, such as at least 10,000-fold lower, for instance at least 100,000-fold lower than its K_{D} of binding to a non-specific antigen (e.g., BSA, casein) other than the predetermined antigen or a closely related antigen. The amount with which the K_{D} of binding is lower is dependent on the K_{D} of the antibody, so that when the K_{D} of the antibody is very low, then the amount with which the K_{D} of binding to the antigen is lower than the K_{D} of binding to a non-specific antigen may be at least 10,000-fold (that is, the antibody is highly specific).

The term “K_{D}” (M), as used herein, refers to the dissociation equilibrium constant of a particular antibody-antigen interaction. Affinity, as used herein, and K_{D} are inversely related, that is that higher affinity is intended to refer to lower K_{D}, and lower affinity is intended to refer to higher K_{D}.

The term “ADC” refers to an antibody-drug conjugate.

The abbreviations “vc” and “val-cit” refer to the dipeptide valine-citrulline.

The abbreviation “PAB” refers to the self-immolative spacer:

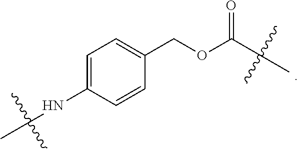 [SMILES: *Nc1ccc(COC(*)=O)cc1]

The abbreviation “MC” refers to the stretcher maleimidocaproyl:

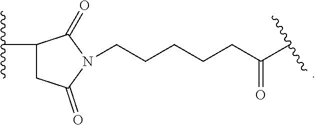 [SMILES: *C(=O)CCCCCN1C(=O)CC(*)C1=O]

The term “Ab-MC-vc-PAB-MMAE” refers to an antibody conjugated to the drug MMAE through a MC-vc-PAB linker.

The term “cAC10-MC-vc-PAB-MMAE” refers to a chimeric AC10 antibody conjugated to the drug MMAE through a MC-vc-PAB linker.

An “anti-CD30 vc-PAB-MMAE antibody-drug conjugate” refers to an anti-CD30 antibody conjugated to the drug MMAE via a linker comprising the dipeptide valine citrulline and the self-immolative spacer PAB as shown in Formula (I) of U.S. Pat. No. 9,211,319.

“CD30” or “TNFRSF8” refers to a receptor that is a member of the tumor necrosis factor receptor superfamily. CD30 is a transmembrane glycoprotein expressed on activated CD4+ and CD8+ T cells and B cells, and virally-infected lymphocytes. CD30 interacts with TRAF2 and TRAF3 to mediate signal transduction that leads to activation of NF-κB. CD30 acts as a positive regulator of apoptosis, and it has been shown to limit the proliferative potential of auto-reactive CD8 effector T cells. CD30 is also expressed by various forms of lymphoma, including Hodgkin lymphoma (CD30 is expressed by Reed-Sternberg cells) and non-Hodgkin lymphoma (e.g., diffuse large B-cell lymphoma (DLBCL), peripheral T-cell lymphoma (PTCL), and cutaneous T-cell lymphoma (CTCL).

“Programmed Death-1” (PD-1) refers to an immunoinhibitory receptor belonging to the CD28 family. PD-1 is expressed predominantly on previously activated T-cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. In some embodiments, hPD-1 comprises the amino acid sequence found under GenBank Accession No. U64863.

“Programmed Death Ligand-1” (PD-L1) is one of two cell surface glycoprotein ligands for PD-1 (the other being PD-L2) that downregulate T-cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. In some embodiments, hPD-L1 comprises the amino acid sequence found under GenBank Accession No. Q9NZQ7.

“Combined positive score” or “CPS” is the ratio of the number of PD-L1 positive tumor cells and PD-L1 positive mononuclear inflammatory cells (MIC) within the tumor nests and the adjacent supporting stroma (numerator) compared to the total number of tumor cells (denominator, i.e., the number of PD-L1 positive and PD-L1 negative tumor cells).

“Tumor proportion score” or “TPS” is the percentage of viable tumor cells showing partial or complete PD-L1 membrane staining in an immunohistochemical assay at any intensity.

A “cancer” refers to a broad group of various diseases characterized by the uncontrolled growth of abnormal cells in the body. A “cancer” or “cancer tissue” can include a tumor. Unregulated cell division and growth results in the formation of malignant tumors that invade neighboring tissues and can also metastasize to distant parts of the body through the lymphatic system or bloodstream. Following metastasis, the distal tumors can be said to be “derived from” the pre-metastasis tumor.

The term “immunotherapy” refers to the treatment of a subject afflicted with, at risk of contracting, or suffering a recurrence of a disease by a method comprising inducing, enhancing, suppressing, or otherwise modifying an immune response.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects).

In other embodiments of the disclosure, tumor regression can be observed and continue for a period of at least about 20 days, at least about 30 days, at least about 40 days, at least about 50 days, or at least about 60 days. Notwithstanding these ultimate measurements of therapeutic effectiveness, evaluation of immunotherapeutic drugs must also make allowance for “immune-related response patterns”.

A therapeutically effective amount of a drug (e.g., anti-CD30 antibody-drug conjugate or anti-PD-1 antibody) includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound (e.g., an anti-CD30 antibody-drug conjugate or anti-PD-1 antibody) that is lower than the usual or typical dose of the therapeutic compound when administered alone for the treatment of a hyperproliferative disease (e.g., cancer).

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “objective response rate” or “ORR” refers to the sum of complete response (CR) rate and partial response (PR) rate.

As used herein, “overall survival” or “OS” refers to the percentage of individuals in a group who are likely to be alive after a particular duration of time.

The term “weight-based dose”, as referred to herein, means that a dose administered to a subject is calculated based on the weight of the subject.

The use of the term “fixed dose” with regard to a method of the disclosure means that two or more different antibodies (e.g., anti-PD-1 antibody and anti-CD30 antibody-drug conjugate) are administered to a subject in particular (fixed) ratios with each other. In some embodiments, the fixed dose is based on the amount (e.g., mg) of the antibodies. In certain embodiments, the fixed dose is based on the concentration (e.g., mg/ml) of the antibodies. For example, a 3:1 ratio of an anti-PD-1 antibody to an anti-CD30 antibody-drug conjugate administered to a subject can mean about 240 mg of the anti-PD-1 antibody and about 80 mg of the anti-CD30 antibody-drug conjugate or about 3 mg/ml of the anti-PD-1 antibody and about 1 mg/ml of the anti-CD30 antibody-drug conjugate are administered to the subject.

The use of the term “flat dose” with regard to the methods and dosages of the disclosure means a dose that is administered to a subject without regard for the weight or body surface area (BSA) of the subject. The flat dose is therefore not provided as a mg/kg dose, but rather as an absolute amount of the agent (e.g., the anti-CD30 antibody-drug conjugate and/or anti-PD-1 antibody). For example, a subject with 60 kg body weight and a subject with 100 kg body weight would receive the same dose of an antibody or an antibody-drug conjugate.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Administering” or “administration” refer to the physical introduction of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion (e.g., intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom. The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

The term “monotherapy” as used herein means that the anti-CD30 antibody-drug conjugate or anti-PD-1 antibody is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event; it does not refer to an event which hypothetically might have caused death if it was more severe.
Results in persistent or significant disability/incapacity
Constitutes a congenital anomaly/birth defect
Is medically significant, i.e., defined as an event that jeopardizes the patient or may require medical or surgical intervention to prevent one of the outcomes listed above. Medical and scientific judgment must be exercised in deciding whether an AE is “medically significant”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±two days, i.e., every twelve days to every sixteen days. “Once about every three weeks” can include every twenty-one days±three days, i.e., every eighteen days to every twenty-four days. Similar approximations apply, for example, to once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

As described herein, any concentration range, percentage range, ratio range, or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Combination Therapy

One aspect of the invention provides anti-CD30 antibody-drug conjugates that binds to CD30 for use in the treatment of cancer wherein the antibody-drug conjugate is for administration, or to be administered in combination with an anti-PD-1 antibody or an antigen-binding fragment thereof, wherein the cancer is a solid tumor. In some embodiments, the solid tumor is selected from the group consisting of non-small cell lung cancer (NSCLC), melanoma, head and neck cancer, renal cell carcinoma, microsatellite instability-high or mismatch repair deficient cancer, bladder cancer, colon cancer, rectal cancer, gastric cancer, gastroesophageal junction carcinoma, esophageal cancer, cervical cancer, hepatocellular carcinoma, endometrial carcinoma, Merkel cell carcinoma, cutaneous squamous cell carcinoma, breast cancer, and tumor mutational burden-high cancer.

### A. Anti-CD30 Antibody

In one aspect, the therapy of the present disclosure utilizes an anti-CD30 antibody or an antigen-binding fragment thereof. CD30 receptors are members of the tumor necrosis factor receptor superfamily involved in limiting the proliferative potential of autoreactive CD8 effector T cells. Antibodies targeting CD30 can potentially be either agonists or antagonists of these CD30 mediated activities. In some embodiments, the anti-CD30 antibody is conjugated to a therapeutic agent (e.g., an anti-CD30 antibody-drug conjugate).

Murine anti-CD30 mAbs known in the art have been generated by immunization of mice with Hodgkin's disease (HD) cell lines or purified CD30 antigen. AC10, originally termed C10 (Bowen et al., 1993, J. Immunol. 151:58965906), is distinct in that this anti-CD30 mAb that was prepared against a hum an NK-like cell line, YT (Bowen et al., 1993, J. Immunol. 151:5896 5906). Initially, the signaling activity of this mAb was evidenced by the down regulation of the cell surface expression of CD28 and CD45 molecules, the up regulation of cell surface CD25 expression and the induction of homotypic adhesion following binding of C10 to YT cells. Sequences of the AC10 antibody are set out in SEQ ID NO: 1-16. See also U.S. Pat. No. 7,090,843, incorporated herein by reference.

Generally, anti-CD30 antibodies of the disclosure bind CD30, e.g., human CD30, and exert cytostatic and cytotoxic effects on cells expressing CD30. Anti-CD30 antibodies of the disclosure are preferably monoclonal, and may be multispecific, human, humanized or chimeric antibodies, single chain antibodies, Fab fragments, F(ab′) fragments, fragments produced by a Fab expression library, and CD30 binding fragments of any of the above. In some embodiments, the anti-CD30 antibodies of the disclosure specifically bind CD30. The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the anti-CD30 antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-CD30 antibodies or antigen-binding fragments thereof are human, murine (e.g., mouse and rat), donkey, sheep, rabbit, goat, guinea pig, camelid, horse, or chicken.

The anti-CD30 antibodies of the present disclosure may be monospecific, bispecific, trispecific or of greater multi specificity. Multispecific antibodies may be specific for different epitopes of CD30 or may be specific for both CD30 as well as for a heterologous protein. See, e.g., PCT publications WO 93/17715; WO 92/08802; WO 91/00360; WO 92/05793; Tutt, et al., 1991, J. Immunol. 147:6069; U.S. Pat. Nos. 4,474,893; 4,714,681; 4,925,648; 5,573,920; 5,601,819; Kostelny et al., 1992, J. Immunol. 148:15471553.

Anti-CD30 antibodies of the present disclosure may be described or specified in terms of the particular CDRs they comprise. In certain embodiments antibodies of the disclosure comprise one or more CDRs of AC10. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Martin et al., “Modeling antibody hypervariable loops: a combined algorithm,” PNAS, 1989, 86 (23): 9268-9272, (“AbM” numbering scheme). The boundaries of a given CDR may vary depending on the scheme used for identification. In some embodiments, a “CDR” or “complementarity determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given VH or VL region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

The disclosure encompasses an antibody or derivative thereof comprising a heavy or light chain variable domain, said variable domain comprising (a) a set of three CDRs, in which said set of CDRs are from monoclonal antibody AC10, and (b) a set of four framework regions, in which said set of framework regions differs from the set of framework regions in monoclonal antibody AC10, and in which said antibody or derivative thereof immunospecifically binds CD30.

In one aspect, the anti-CD30 antibody is AC10. In some embodiments, the anti-CD30 antibody is cAC10. cAC10 is a chimeric IgG1 monoclonal antibody that specifically binds CD30. cAC10 induces growth arrest of CD30⁺ cell lines in vitro and has pronounced antitumor activity in severe combined immunodeficiency (SCID) mouse xenograft models of Hodgkin disease. See Francisco et al., Blood 102 (4): 1458-64 (2003). AC10 antibody and cAC10 antibody are described in U.S. Pat. Nos. 9,211,319 and 7,090,843.

In one aspect, anti-CD30 antibodies that compete with AC10 antibody and/or cAC10 antibody binding to CD30 are provided. Anti-CD30 antibodies that bind to the same epitope as AC10 antibody and cAC10 antibody are also provided.

In one aspect, provided herein is an anti-CD30 antibody comprising 1, 2, 3, 4, 5, or 6 of the CDR sequences of the AC10 antibody. In one aspect, provided herein is an anti-CD30 antibody comprising 1, 2, 3, 4, 5, or 6 of the CDR sequences of the cAC10 antibody. In some embodiments, the CDR is a Kabat CDR or a Chothia CDR.

In one aspect, provided herein is an anti-CD30 antibody comprising a heavy chain variable region and a light chain variable region, wherein the heavy chain variable region comprises (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3; and/or wherein the light chain variable region comprises (i) CDR-L1 comprising the amino acid sequence of SEQ ID NO:4, (ii) CDR-L2 comprising the amino acid sequence of SEQ ID NO:5, and (iii) CDR-L3 comprising the amino acid sequence of SEQ ID NO:6.

An anti-CD30 antibody described herein may comprise any suitable framework variable domain sequence, provided that the antibody retains the ability to bind CD30 (e.g., human CD30). As used herein, heavy chain framework regions are designated “HC-FR1-FR4,” and light chain framework regions are designated “LC-FR1-FR4.” In some embodiments, the anti-CD30 antibody comprises a heavy chain variable domain framework sequence of SEQ ID NO:9, 10, 11, and 12 (HC-FR1, HC-FR2, HC-FR3, and HC-FR4, respectively). In some embodiments, the anti-CD30 antibody comprises a light chain variable domain framework sequence of SEQ ID NO: 13, 14, 15, and 16 (LC-FR1, LC-FR2, LC-FR3, and LC-FR4, respectively).

In one embodiment, an anti-CD30 antibody comprises a heavy chain variable domain comprising a framework sequence and hypervariable regions, wherein the framework sequence comprises the HC-FR1-HC-FR4 amino acid sequences of SEQ ID NO:9 (HC-FR1), SEQ ID NO:10 (HC-FR2), SEQ ID NO:11 (HC-FR3), and SEQ ID NO:12 (HC-FR4), respectively; the CDR-H1 comprises the amino acid sequence of SEQ ID NO:1; the CDR-H2 comprises the amino acid sequence of SEQ ID NO:2; and the CDR-H3 comprises the amino acid sequence of SEQ ID NO:3.

In one embodiment, an anti-CD30 antibody comprises a light chain variable domain comprising a framework sequence and hypervariable regions, wherein the framework sequence comprises the LC-FR1-LC-FR4 amino acid sequences of SEQ ID NO:13 (LC-FR1), SEQ ID NO: 14 (LC-FR2), SEQ ID NO:15 (LC-FR3), and SEQ ID NO:16 (LC-FR4), respectively; the CDR-L1 comprises the amino acid sequence of SEQ ID NO:4; the CDR-L2 comprises the amino acid sequence of SEQ ID NO:5; and the CDR-L3 comprises the amino acid sequence of SEQ ID NO:6.

In some embodiments of the anti-CD30 antibodies described herein, the heavy chain variable domain comprises the amino acid sequence of QIQLQQSGPEVVKPGASVKISCKASGYTFTDYYITWVKQKPGQGLEWIGWIYPGSGN TKYNEKFKGKATLTVDTSSSTAFMQLSSLTSEDTAVYFCANYGNYWFAYWGQGTQ VTVSA (SEQ ID NO:7) and the light chain variable domain comprises the amino acid sequence of

(SEQ ID NO: 8)
DIVLTQSPASLAVSLGQRATISCKASQSVDFDGDSYMNWYQQKPGQPPK
VLIYAASNLESGIPARFSGSGSGTDFTLNIHPVEEEDAATYYCQQSNED
PWTFGGGTKLEIK.

In some embodiments of the anti-CD30 antibodies described herein, the heavy chain CDR sequences comprise the following:

a) CDR-H1;
(DYYIT (SEQ ID NO: 1))
b) CDR-H2
(WIYPGSGNTKYNEKFKG (SEQ ID NO: 2));
and
c) CDR-H3
(YGNYWFAY (SEQ ID NO: 3)).

In some embodiments of the anti-CD30 antibodies described herein, the heavy chain FR sequences comprise the following:

a) HC-FR1
(QIQLQQSGPEVVKPGASVKISCKASGYTFT (SEQ ID NO: 9));
b) HC-FR2
(WVKQKPGQGLEWIG (SEQ ID NO: 10));
c) HC-FR3
(KATLTVDTSSSTAFMQLSSLTSEDTAVYFCAN (SEQ ID
NO: 11));
and
d) HC-FR4
(WGQGTQVTVSA (SEQ ID NO: 12)).

In some embodiments of the anti-CD30 antibodies described herein, the light chain CDR sequences comprise the following:

a) CDR-L1
(KASQSVDFDGDSYMN (SEQ ID NO: 4));
b) CDR-L2
(AASNLES (SEQ ID NO: 5));
and
c) CDR-L3
(QQSNEDPWT (SEQ ID NO: 6)).

In some embodiments of the anti-CD30 antibodies described herein, the light chain FR sequences comprise the following:

a) LC-FR1
(DIVLTQSPASLAVSLGQRATISC (SEQ ID NO: 13));
b) LC-FR2
(WYQQKPGQPPKVLIY (SEQ ID NO: 14));
c) LC-FR3
(GIPARFSGSGSGTDFTLNIHPVEEEDAATYYC (SEQ ID
NO: 15));
and
d) LC-FR4
(FGGGTKLEIK (SEQ ID NO: 16)).

In some embodiments, provided herein is an anti-CD30 antibody that binds to CD30 (e.g., human CD30), wherein the antibody comprises a heavy chain variable region and a light chain variable region, wherein the antibody comprises:
(a) heavy chain variable domain comprising:
   (1) an HC-FR1 comprising the amino acid sequence of SEQ ID NO:9;
   (2) an CDR-H1 comprising the amino acid sequence of SEQ ID NO:1;
   (3) an HC-FR2 comprising the amino acid sequence of SEQ ID NO:10;
   (4) an CDR-H2 comprising the amino acid sequence of SEQ ID NO:2;
   (5) an HC-FR3 comprising the amino acid sequence of SEQ ID NO:11;
   (6) an CDR-H3 comprising the amino acid sequence of SEQ ID NO:3; and
   (7) an HC-FR4 comprising the amino acid sequence of SEQ ID NO:12, and/or
(b) a light chain variable domain comprising:
   (1) an LC-FR1 comprising the amino acid sequence of SEQ ID NO:13;
   (2) an CDR-L1 comprising the amino acid sequence of SEQ ID NO:4;
   (3) an LC-FR2 comprising the amino acid sequence of SEQ ID NO:14;
   (4) an CDR-L2 comprising the amino acid sequence of SEQ ID NO:5;
   (5) an LC-FR3 comprising the amino acid sequence of SEQ ID NO:15;
   (6) an CDR-L3 comprising the amino acid sequence of SEQ ID NO:6; and
   (7) an LC-FR4 comprising the amino acid sequence of SEQ ID NO:16.

In one aspect, provided herein is an anti-CD30 antibody comprising a heavy chain variable domain comprising the amino acid sequence of SEQ ID NO:7 and/or comprising a light chain variable domain comprising the amino acid sequence of SEQ ID NO:8.

In some embodiments, provided herein is an anti-CD30 antibody comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:7 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:7. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a heavy chain variable domain sequence of SEQ ID NO:7 including post-translational modifications of that sequence. In a particular embodiment, the heavy chain variable domain comprises one, two or three CDRs selected from: (a) CDR-H1 comprising the amino acid sequence of SEQ ID NO: 1, (b) CDR-H2 comprising the amino acid sequence of SEQ ID NO:2, and (c) CDR-H3 comprising the amino acid sequence of SEQ ID NO:3.

In some embodiments, provided herein is an anti-CD30 antibody comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO: 8. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO: 8 contains substitutions (e.g., conservative substitutions), insertions, or deletions relative to the reference sequence and retains the ability to bind to a CD30 (e.g., human CD30). In certain embodiments, a total of 1 to 10 amino acids have been substituted, inserted and/or deleted in SEQ ID NO:8. In certain embodiments, substitutions, insertions, or deletions (e.g., 1, 2, 3, 4, or 5 amino acids) occur in regions outside the CDR s (i.e., in the FRs). In some embodiments, the anti-CD30 antibody comprises a light chain variable domain sequence of SEQ ID NO: 8 including post-translational modifications of that sequence. In a particular embodiment, the light chain variable domain comprises one, two or three CDRs selected from: (a) CDR-H1 comprising the amino acid sequence of SEQ ID NO:4, (b) CDR-H2 comprising the amino acid sequence of SEQ ID NO: 5, and (c) CDR-H3 comprising the amino acid sequence of SEQ ID NO: 6.

In some embodiments, the anti-CD30 antibody comprises a heavy chain variable domain as in any of the embodiments provided above, and a light chain variable domain as in any of the embodiments provided above. In one embodiment, the antibody comprises the heavy chain variable domain sequence of SEQ ID NO:7 and the light chain variable domain sequence of SEQ ID NO:8, including post-translational modifications of those sequences.

In some embodiments, the anti-CD30 antibody of the anti-CD30 antibody-drug conjugate comprises: i) a heavy chain CDR1 set out in SEQ ID NO: 1, a heavy chain CDR2 set out in SEQ ID NO: 2, a heavy chain CDR3 set out in SEQ ID NO: 3; and ii) a light chain CDR1 set out in SEQ ID NO: 4, a light chain CDR2 set out in SEQ ID NO: 5, and a light chain CDR3 set out in SEQ ID NO: 6.

In some embodiments, the anti-CD30 antibody of the anti-CD30 antibody-drug conjugate comprises: i) an amino acid sequence at least 85% identical to a heavy chain variable region set out in SEQ ID NO: 7, and ii) an amino acid sequence at least 85% identical to a light chain variable region set out in SEQ ID NO: 8.

In some embodiments, the anti-CD30 antibody of the anti-CD30 antibody-drug conjugate is a monoclonal antibody.

In some embodiments, the anti-CD30 antibody of the anti-CD30 antibody-drug conjugate is a chimeric AC10 antibody.

In some embodiments, the anti-CD30 antibody of the anti-CD30 antibody-drug conjugate is brentuximab or a biosimilar thereof. In some embodiments, the anti-CD30 antibody of the anti-CD30 antibody-drug conjugate is brentuximab.

Antibodies of the present invention may also be described or specified in terms of their binding affinity to CD30. Preferred binding affinities include those with a dissociation constant or Kd less than 5×10² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

In one aspect of the invention, polynucleotides encoding anti-CD30 antibodies, such as those anti-CD30 antibodies described herein, are provided. In certain embodiments, vectors comprising polynucleotides encoding anti-CD30 antibodies as described herein are provided. In certain embodiments, host cells comprising such vectors are provided. In another aspect of the invention, compositions comprising anti-CD30 antibodies described herein or polynucleotides encoding anti-CD30 antibodies described herein are provided.

The antibodies also include derivatives that are modified, i.e., by the covalent attachment of any type of molecule to the antibody such that covalent attachment does not prevent the antibody from binding to CD30 or from exerting a cytostatic or cytotoxic effect on HD cells. For example, but not by way of limitation, the antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### B. Antibody-Drug Conjugate Structure

In some embodiments, the anti-CD30 antibody is conjugated to a therapeutic agent (e.g., an anti-CD30 antibody-drug conjugate). In some embodiments, the therapeutic agent comprises an anti-neoplastic agent (e.g., an anti-mitotic agent). In certain embodiments, the therapeutic agent is an auristatin. In certain embodiments, the therapeutic agent is selected from the group consisting of monomethyl auristatin E (MMAE), monomethyl auristatin F (MMAF), auristatin drug analogues, cantansinoids, maytansinoids (e.g., maytansine; DMs), dolastatins, cryptophycin, duocarmycin, duocarmycin derivatives, esperamicin, calicheamicin, pyrolobenodiazepine (PBD), and any combination thereof. In some embodiments, the therapeutic agent is a substrate of an efflux pump which requires the expression of MDR1. MMAE is a substrate of an efflux pump which requires the expression of MDR1. In some embodiments, the therapeutic agent has increased cytotoxicity for T regulatory cells compared to other T cell populations. In one particular embodiment, the anti-CD30 antibody is conjugated to MMAE. The antibody can be conjugated to at least one, at least two, at least three, at least four, at least five, at least six, at least seven, at least eight, at least nine, or at least ten molecules of the therapeutic agent (e.g., MMAE). In one embodiment, the anti-CD30 antibody is conjugated to four molecules of the therapeutic agent, e.g., four molecules of MMAE. In one particular embodiment, the anti-CD30 antibody is conjugated to MMA The antibody can be conjugated to at least one, at least two, at least three, at least four, at least five, at least six, at least seven, at least eight, at least nine, or at least ten molecules of the therapeutic agent (e.g., MMAF). In one embodiment, the anti-CD30 antibody is conjugated to four molecules of the therapeutic agent, e.g., four molecules of MMAF.

In one embodiment, the auristatin is monomethyl auristatin E (MMAE):

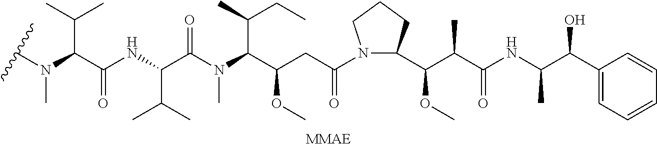 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the attachment site for the linker.

In one embodiment, the auristatin is monomethyl auristatin F (MMAF):

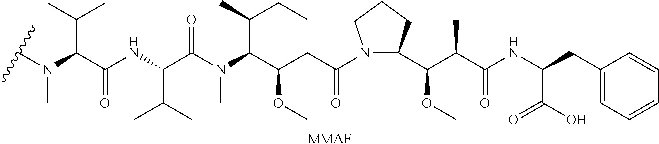

wherein the wavy line indicates the attachment site for the linker.

In some embodiments, the anti-CD30 antibody-drug conjugate further comprises a linker between the therapeutic agent and the antibody. In some embodiments, the linker comprises one or more naturally occurring amino acids, one or more non-naturally occurring (e.g., synthetic) amino acids, a chemical linker, or any combination thereof. In certain embodiments, the linker is a cleavable linker, e.g., a protease cleavable linker. In certain embodiments, the linker is specifically cleaved upon uptake by a target cell, e.g., upon uptake by a cell expressing CD30. In certain embodiments, the linker is a cleavable peptide linker having the formula: “-MC-vc-PAB-” or “-MC-val-cit-PAB-”, wherein “MC” refers to the stretcher maleimidocaproyl having the following structure:

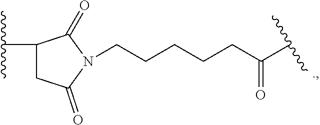 [SMILES: *C(=O)CCCCCN1C(=O)CC(*)C1=O]

“vc” and “val-cit” refer to the dipeptide valine-citrulline, and PAB refers to a self-immolative spacer having the following structure:

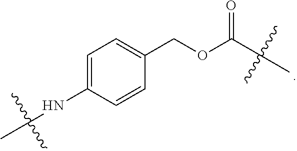 [SMILES: *Nc1ccc(COC(*)=O)cc1]

In some embodiments, cleavage of the linker activates a cytotoxic activity of the therapeutic agent. In certain embodiments, the linker is a non-cleavable linker. In certain embodiments, the non-cleavable linker has the formula: “-MC-”, wherein “MC” refers to the stretcher maleimidocaproyl having the following structure:

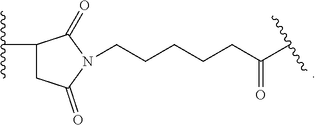 [SMILES: *C(=O)CCCCCN1C(=O)CC(*)C1=O]

In some embodiments, the antibody-drug conjugate comprises an anti-CD30 antibody, covalently linked to MMAE through a vc-PAB linker. In some embodiments, the antibody-drug conjugate is delivered to the subject as a pharmaceutical composition. In some embodiments, the CD30 antibody-drug conjugates contemplated herein are as described in U.S. Pat. No. 9,211,319, herein incorporated by reference.

In one embodiment, the anti-CD30 antibody-drug conjugate comprises brentuximab vedotin. In one particular embodiment, the anti-CD30 antibody-drug conjugate is brentuximab vedotin or a biosimilar thereof. In one particular embodiment, the anti-CD30 antibody-drug conjugate is brentuximab vedotin. Brentuximab vedotin (BV; also known as “ADCETRIS®”) is a CD30-directed antibody-drug conjugate (ADC) comprising a chimeric anti-CD30 antibody (cAC10), a therapeutic agent (MMAE), and a protease-cleavable linker between the cAC10 and the MMAE, as shown in the following structure:

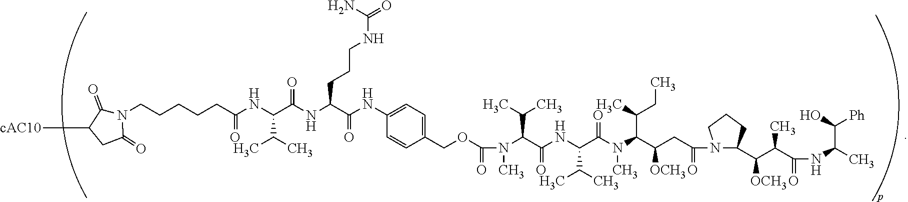

The drug to antibody ratio or drug loading is represented by “p” in the structure of brentuximab vedotin and ranges in integer values from 1 to 8. The average drug loading of brentuximab vedotin in a pharmaceutical composition is about 4. ADCETRIS® is approved by the FDA for treatment of patients with Hodgkin lymphoma after failure of autologous stem cell transplant (ASCT) or after failure of at least two prior multi-agent chemotherapy regimens in patients who are not ASCT candidates and for the treatment of patients with systemic anaplastic large cell lymphoma after failure of at least one prior multi-agent chemotherapy regimen. The mechanisms of action of brentuximab vedotin and MMAE, including the effects on various T cell populations and immunogenic cell death, such as T regulatory cells, have been reported. See WO2019/183438 and WO2019/075188.

In one embodiment, the anti-CD30 antibody is an anti-CD30 antibody or antigen binding fragment thereof that binds to the same epitope as cAC10, e.g., the same epitope as brentuximab vedotin. In certain embodiments, the anti-CD30 antibody is an antibody that has the same CDRs as cAC10, e.g., the same CDRs as brentuximab vedotin. Antibodies that bind to the same epitope are expected to have functional properties very similar to those of cAC10 by virtue of their binding to the same epitope region of CD30. These antibodies can be readily identified based on their ability to, for example, cross-compete with cAC10 in standard CD30 binding assays such as Biacore analysis, ELISA assays, or flow cytometry.

In certain embodiments, the antibodies that cross-compete for binding to human CD30 with or bind to the same epitope region of human CD30 as cAC10 are monoclonal antibodies. For administration to human subjects, these cross-competing antibodies can be chimeric antibodies, or can be humanized or human antibodies. Such chimeric, humanized, or human monoclonal antibodies can be prepared and isolated by methods well known in the art. Anti-CD30 antibodies usable in the methods of the disclosed disclosure also include antigen binding portions of the above antibodies.

In other embodiments, the anti-CD30 antibody or antigen-binding portion thereof is a chimeric, humanized, or human monoclonal antibody or a portion thereof. In certain embodiments for treating a human subject, the antibody is a humanized antibody. In other embodiments for treating a human subject, the antibody is a human antibody. Antibodies of an IgG1, IgG2, IgG3, or IgG4 isotype can be used.

In one embodiment, the antibody-drug conjugate is brentuximab vedotin or a biosimilar thereof. In one embodiment, the antibody-drug conjugate is brentuximab vedotin.

### C. Anti-PD-1 Antibody

In one aspect, the therapy of the present disclosure utilizes an anti-PD-1 antibody or an antigen binding fragment thereof.

Generally, anti-PD-1 antibodies or antigen-binding fragments thereof of the disclosure bind to PD-1, e.g., human PD-1. Anti-PD-1 antibodies of the disclosure are preferably monoclonal and may be multispecific, human, humanized or chimeric antibodies, single chain antibodies, Fab fragments, F(ab′) fragments, fragments produced by a Fab expression library, and PD-1 binding fragments of any of the above. In some embodiments, an anti-PD-1 antibody described herein comprises the CDRs of pembrolizumab and binds specifically to PD-1 (e.g., human PD-1). The immunoglobulin molecules of the disclosure can be of any type (e.g., IgG, IgE, IgM, IgD, IgA and IgY), class (e.g., IgG1, IgG2, IgG3, IgG4, IgA1 and IgA2) or subclass of immunoglobulin molecule.

In certain embodiments of the disclosure, the antibodies are antigen-binding fragments (e.g., human antigen-binding fragments) as described herein and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains. In some embodiments, the anti-PD-1 antibodies or antigen-binding fragments thereof are human, murine (e.g., mouse and rat), donkey, sheep, rabbit, goat, guinea pig, camelid, horse, or chicken

The anti-PD-1 antibodies of the present disclosure may be monospecific, bispecific, trispecific or of greater multi specificity. Multispecific antibodies may be specific for different epitopes of PD-1 or may be specific for both PD-1 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; Kostelny et al., 1992, J. Immunol. 148:1547 1553.

Anti-PD-1 antibodies of the present disclosure may be described or specified in terms of the particular CDRs they comprise. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Martin et al., “Modeling antibody hypervariable loops: a combined algorithm,” PNAS, 1989, 86 (23): 9268-9272, (“AbM” numbering scheme). The boundaries of a given CDR may vary depending on the scheme used for identification. In some embodiments, a “CDR” or “complementary determining region,” or individual specified CDRs (e.g., CDR-H1, CDR-H2, CDR-H3), of a given antibody or region thereof (e.g., variable region thereof) should be understood to encompass a (or the specific) CDR as defined by any of the aforementioned schemes. For example, where it is stated that a particular CDR (e.g., a CDR-H3) contains the amino acid sequence of a corresponding CDR in a given V_{H} Or V_{L} region amino acid sequence, it is understood that such a CDR has a sequence of the corresponding CDR (e.g., CDR-H3) within the variable region, as defined by any of the aforementioned schemes. The scheme for identification of a particular CDR or CDRs may be specified, such as the CDR as defined by the Kabat, Chothia, AbM or IMGT method.

Numbering of amino acid residues in CDR sequences of the anti-PD-1 antibodies and antigen-binging fragments provided herein are generally according to the Kabat numbering scheme as described in Kabat E. A., et al., 1991, Sequences of proteins of Immunological interest, In: NIH Publication No. 91-3242, US Department of Health and Human Services, Bethesda, MD.

In some embodiments, anti-PD-1 antibodies or antigen-binding fragments thereof of the present disclosure comprise the complementarity determining regions (CDRs) of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003, or a biosimilar thereof. In some embodiments, anti-PD-1 antibodies or antigen-binding fragments thereof of the present disclosure comprise the complementarity determining regions (CDRs) of an antibody or antigen-binding fragment selected from the group consisting of pembrolizumab, nivolumab, Amp-514, tislelizumab, cemiplimab, TSR-042, JNJ-63723283, CBT-501, PF-06801591, JS-001, camrelizumab, PDR001, BCD-100, AGEN2034, IBI-308, BI-754091, GLS-010, LZM-009, AK-103, MGA-012, Sym-021 and CS1003.

In some embodiments, anti-PD-1 antibodies or antigen-binding fragments thereof of the present disclosure comprise the CDRs of the antibody pembrolizumab. See U.S. Pat. Nos. 8,354,509 and 8,900,587. The present disclosure encompasses an anti-PD-1 antibody or derivative thereof comprising a heavy or light chain variable domain, said variable domain comprising (a) a set of three CDRs, in which said set of CDRs are from the monoclonal antibody pembrolizumab, and (b) a set of four framework regions, in which said set of framework regions differs from the set of framework regions in the monoclonal antibody pembrolizumab, and in which said anti-PD-1 antibody or derivative thereof binds to PD-1. In certain embodiments, the anti-PD-1 antibody is pembrolizumab. The antibody pembrolizumab is also known as KEYTRUDA®. (Merck & Co., Inc., Kenilworth, NJ, USA).

In one aspect, provided herein is an anti-PD-1 antibody or antigen-binding fragment thereof comprising a heavy chain variable region and a light chain variable region, wherein the heavy chain variable region comprises (i) CDR-H1 comprising the amino acid sequence of SEQ ID NO:17, (ii) CDR-H2 comprising the amino acid sequence of SEQ ID NO: 18, and (iii) CDR-H3 comprising the amino acid sequence of SEQ ID NO:19; and wherein the light chain variable region comprises (i) CDR-L1 comprising the amino acid sequence of SEQ ID NO:20, (ii) CDR-L2 comprising the amino acid sequence of SEQ ID NO: 21, and (iii) CDR-L3 comprising the amino acid sequence of SEQ ID NO:22, wherein the CDRs of the anti-PD-1 antibody are generally defined by the Kabat numbering scheme.

In one embodiment, an anti-PD-1 antibody or antigen-binding fragment thereof comprises a heavy chain variable domain comprising a framework sequence and hypervariable regions, wherein the framework sequence comprises the HC-FR1-HC-FR4 amino acid sequences of SEQ ID NO:23 (HC-FR1), SEQ ID NO:24 (HC-FR2), SEQ ID NO: 25 (HC-FR3), and SEQ ID NO:26 (HC-FR4), respectively; the CDR-H1 comprises the amino acid sequence of SEQ ID NO:17; the CDR-H2 comprises the amino acid sequence of SEQ ID NO:18; and the CDR-H3 comprises the amino acid sequence of SEQ ID NO:19.

In one embodiment, an anti-PD-1 antibody or antigen-binding fragment thereof comprises a light chain variable domain comprising a framework sequence and hypervariable regions, wherein the framework sequence comprises the LC-FR1-LC-FR4 amino acid sequences of SEQ ID NO:27 (LC-FR1), SEQ ID NO:28 (LC-FR2), SEQ ID NO:29 (LC-FR3), and SEQ ID NO:30 (LC-FR4), respectively; the CDR-L1 comprises the amino acid sequence of SEQ ID NO:20; the CDR-L2 comprises the amino acid sequence of SEQ ID NO: 21; and the CDR-L3 comprises the amino acid sequence of SEQ ID NO:22.

In some embodiments of the anti-PD-1 antibodies or antigen-binding fragments thereof described herein, the heavy chain variable domain comprises the amino acid sequence of QVQLVQSGVEVKKPGASVKVSCKASGYTFTNYYMYWVRQAPGQGLEWMGGINPS NGGTNFNEKFKNRVTLTTDSSTTTAYMELKSLQFDDTAVYYCARRDYRFDMGFDY WGQGTTVTVSS (SEQ ID NO:31) and the light chain variable domain comprises the amino acid sequence of

(SEQ ID NO: 32)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIK.

In some embodiments of the anti-PD-1 antibodies or antigen-binding fragments thereof described herein, the heavy chain CDR sequences comprise the following:

a) CDR-H1
(NYYMY (SEQ ID NO: 17));
b) CDR-H2
(GINPSNGGTNFNEKFKN (SEQ ID NO: 18));
and
c) CDR-H3
(RDYRFDMGFDY (SEQ ID NO: 19)).

In some embodiments of the anti-PD-1 antibodies or antigen-binding fragments thereof described herein, the heavy chain FR sequences comprise the following:

a) HC-FR1
(QVQLVQSGVEVKKPGASVKVSCKASGYTFT (SEQ ID NO: 23));
b) HC-FR2
(WVRQAPGQGLEWMG (SEQ ID NO: 24));
c) HC-FR3
(RVTLTTDSSTTTAYMELKSLQFDDTAVYYCAR (SEQ ID
NO: 25));
and
d) HC-FR4
(WGQGTTVTVSS (SEQ ID NO: 26)).

In some embodiments of the anti-PD-1 antibodies or antigen-binding fragments thereof described herein, the light chain CDR sequences comprise the following:

a) CDR-L1
(RASKGVSTSGYSYLH (SEQ ID NO: 20));
b) CDR-L2
(LASYLES (SEQ ID NO: 21));
and
c) CDR-L3
(QHSRDLPLT (SEQ ID NO: 22)).

In some embodiments of the anti-PD-1 antibodies or antigen-binding fragments thereof described herein, the light chain FR sequences comprise the following:

a) LC-FR1
(EIVLTQSPATLSLSPGERATLSC (SEQ ID NO: 27));
b) LC-FR2
(WYQQKPGQAPRLLIY (SEQ ID NO: 28));
c) LC-FR3
(GVPARFSGSGSGTDFTLTISSLEPEDFAVYYC (SEQ ID
NO: 29));
and
d) LC-FR4
(FGGGTKVEIK (SEQ ID NO: 30)).

In some embodiments, provided herein is an anti-PD-1 antibody or antigen-binding fragment thereof that binds to PD-1 (e.g., human PD-1), wherein the antibody comprises a heavy chain variable region and a light chain variable region, wherein the antibody comprises:
(a) heavy chain variable domain comprising:
   (1) an HC-FR1 comprising the amino acid sequence of SEQ ID NO:23;
   (2) an CDR-H1 comprising the amino acid sequence of SEQ ID NO:17;
   (3) an HC-FR2 comprising the amino acid sequence of SEQ ID NO:24;
   (4) an CDR-H2 comprising the amino acid sequence of SEQ ID NO:18;
   (5) an HC-FR3 comprising the amino acid sequence of SEQ ID NO:25;
   (6) an CDR-H3 comprising the amino acid sequence of SEQ ID NO:19; and
   (7) an HC-FR4 comprising the amino acid sequence of SEQ ID NO:26, and/or
(b) a light chain variable domain comprising:
   (1) an LC-FR1 comprising the amino acid sequence of SEQ ID NO:27;
   (2) an CDR-L1 comprising the amino acid sequence of SEQ ID NO:20;
   (3) an LC-FR2 comprising the amino acid sequence of SEQ ID NO:28;
   (4) an CDR-L2 comprising the amino acid sequence of SEQ ID NO:21;
   (5) an LC-FR3 comprising the amino acid sequence of SEQ ID NO:29;
   (6) an CDR-L3 comprising the amino acid sequence of SEQ ID NO:22; and
   (7) an LC-FR4 comprising the amino acid sequence of SEQ ID NO:30.

In one aspect, provided herein is an anti-PD-1 antibody or antigen-binding fragment thereof comprising a heavy chain variable domain comprising the amino acid sequence of SEQ ID NO:31 or comprising a light chain variable domain comprising the amino acid sequence of SEQ ID NO:32. In one aspect, provided herein is an anti-PD-1 antibody or antigen-binding fragment thereof comprising a heavy chain variable domain comprising the amino acid sequence of SEQ ID NO:31 and comprising a light chain variable domain comprising the amino acid sequence of SEQ ID NO:32. In one aspect, provided herein is an anti-PD-1 antibody or antigen-binding fragment thereof comprising the CDRs of the heavy chain variable domain comprising the amino acid sequence of SEQ ID NO:31 and comprising the CDRs of the light chain variable domain comprising the amino acid sequence of SEQ ID NO:32.

In some embodiments, provided herein is an anti-PD-1 antibody or antigen-binding fragment thereof comprising a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:31. In certain embodiments, a heavy chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:31 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:31. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody or antigen-binding fragment thereof comprises a heavy chain variable domain sequence of SEQ ID NO:31 including post-translational modifications of that sequence. In a particular embodiment, the heavy chain variable domain comprises: (a) CDR-H1 comprising the amino acid sequence of SEQ ID NO: 17, (b) CDR-H2 comprising the amino acid sequence of SEQ ID NO: 18, and (c) CDR-H3 comprising the amino acid sequence of SEQ ID NO:19.

In some embodiments, provided herein is an anti-PD-1 antibody or antigen-binding fragment thereof comprising a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:32. In certain embodiments, a light chain variable domain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:32 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:32. In certain embodiments, substitutions, insertions, or deletions (e.g., 1, 2, 3, 4, or 5 amino acids) occur in regions outside the CDR s (i.e., in the FRs). In some embodiments, the anti-PD-1 antibody or antigen-binding fragment thereof comprises a light chain variable domain sequence of SEQ ID NO:32 including post-translational modifications of that sequence. In a particular embodiment, the light chain variable domain comprises: (a) CDR-L1 comprising the amino acid sequence of SEQ ID NO:20, (b) CDR-L2 comprising the amino acid sequence of SEQ ID NO:21, and (c) CDR-L3 comprising the amino acid sequence of SEQ ID NO:22.

In some embodiments, the anti-PD-1 antibody or antigen-binding fragment thereof comprises a heavy chain variable domain as in any of the embodiments provided above, and a light chain variable domain as in any of the embodiments provided above. In one embodiment, the antibody comprises the heavy chain variable domain sequence of SEQ ID NO: 31 and the light chain variable domain sequence of SEQ ID NO:32, including post-translational modifications of those sequences.

In some embodiments, the anti-PD-1 antibody or antigen-binding fragment thereof comprises: i) a heavy chain CDR1 comprising the amino acid sequence of SEQ ID NO: 17, a heavy chain CDR2 comprising the amino acid sequence of SEQ ID NO: 18, a heavy chain CDR3 comprising the amino acid sequence of SEQ ID NO: 19; and ii) a light chain CDR1 comprising the amino acid sequence of SEQ ID NO: 20, a light chain CDR2 comprising the amino acid sequence of SEQ ID NO: 21, and a light chain CDR3 comprising the amino acid sequence of SEQ ID NO: 22, wherein the CDRs of the anti-PD-1 antibody are generally defined by the Kabat numbering scheme.

In some embodiments, the anti-PD-1 antibody or antigen-binding fragment thereof comprises: i) an amino acid sequence having at least 85% sequence identity to a heavy chain variable region comprising the amino acid sequence of SEQ ID NO: 31, and ii) an amino acid sequence having at least 85% sequence identity to a light chain variable region comprising the amino acid sequence of SEQ ID NO: 32.

In some embodiments, the anti-PD-1 antibody comprises a heavy chain comprising the amino acid sequence of QVQLVQSGVEVKKPGASVKVSCKASGYTFTNYYMYWVRQAPGQGLEWMGGINPS NGGTNFNEKFKNRVTLTTDSSTTTAYMELKSLQFDDTAVYYCARRDYRFDMGFDY WGQGTTVTVSSASTKGPSVFPLAPCSRSTSESTAALGCLVKDYFPEPVTVSWNSGAL TSGVHTFPAVLQSSGLYSLSSVVTVPSSSLGTKTYTCNVDHKPSNTKVDKRVESKYG PPCPPCPAPEFLGGPSVFLFPPKPKDTLMISRTPEVTCVVVDVSQEDPEVQFNWYVDG VEVHNAKTKPREEQFNSTYRVVSVLTVLHQDWLNGKEYKCKVSNKGLPSSIEKTISK AKGQPREPQVYTLPPSQEEMTKNQVSLTCLVKGFYPSDIAVEWESNGQPENNYKTTP PVLDSDGSFFLYSRLTVDKSRWQEGNVFSCSVMHEALHNHYTQKSLSLSLG (SEQ ID NO: 33) and a light chain comprising the amino acid sequence of

(SEQ ID NO: 34)
EIVLTQSPATLSLSPGERATLSCRASKGVSTSGYSYLHWYQQKPGQAPR
LLIYLASYLESGVPARFSGSGSGTDFTLTISSLEPEDFAVYYCQHSRDL
PLTFGGGTKVEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

In some embodiments, provided herein is an anti-PD-1 antibody comprising a heavy chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:33. In certain embodiments, a heavy chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO: 33 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:33. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody comprises a heavy chain sequence of SEQ ID NO: 33 including post-translational modifications of that sequence. In a particular embodiment, the heavy chain comprises: (a) CDR-H1 comprising the amino acid sequence of SEQ ID NO:17, (b) CDR-H2 comprising the amino acid sequence of SEQ ID NO:18, and (c) CDR-H3 comprising the amino acid sequence of SEQ ID NO:19.

In some embodiments, provided herein is an anti-PD-1 antibody comprising a light chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:34. In certain embodiments, a light chain comprising an amino acid sequence having at least 85%, 86%, 87%, 88%, 89%, 90%, 91%, 92%, 93%, 94%, 95%, 96%, 97%, 98%, or 99% sequence identity to the amino acid sequence of SEQ ID NO:34 contains substitutions (e.g., conservative substitutions), insertions, or deletions relative to the reference sequence and retains the ability to bind to a PD-1 (e.g., human PD-1). In certain embodiments, a total of 1 to 10 amino acids have been substituted, inserted and/or deleted in SEQ ID NO:34. In certain embodiments, substitutions, insertions, or deletions (e.g., 1, 2, 3, 4, or 5 amino acids) occur in regions outside the CDRs (i.e., in the FRs). In some embodiments, the anti-PD-1 antibody comprises a light chain sequence of SEQ ID NO:34 including post-translational modifications of that sequence. In a particular embodiment, the light chain comprises: (a) CDR-L1 comprising the amino acid sequence of SEQ ID NO:20, (b) CDR-L2 comprising the amino acid sequence of SEQ ID NO:21, and (c) CDR-L3 comprising the amino acid sequence of SEQ ID NO:22.

In some embodiments, the anti-PD-1 antibody or antigen-binding fragment thereof comprises the CDRs of pembrolizumab and is a monoclonal antibody.

In some embodiments, the anti-PD-1 antibody or antigen-binding fragment thereof inhibits the activity of PD-1.

In some embodiments, the anti-PD-1 antibody or antigen-binding fragment thereof is pembrolizumab, or a biosimilar thereof. In some embodiments, the anti-PD-1 antibody or antigen-binding fragment thereof is pembrolizumab, which is also known as antibody KEYTRUDA® as described in U.S. Pat. Nos. 8,354,509 and 8,900,587.

Anti-PD-1 antibodies or antigen-binding fragments thereof of the invention comprising the CDRs of pembrolizumab may also be described or specified in terms of their binding affinity to PD-1 (e.g., human PD-1). Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

There are five classes of immunoglobulins: IgA, IgD, IgE, IgG and IgM, having heavy chains designated α, δ, ε, γ and μ, respectively. The γ and α classes are further divided into subclasses, e.g., humans express the following subclasses: IgG1, IgG2, IgG3, IgG4, IgA1 and IgA2. IgG1 antibodies can exist in multiple polymorphic variants termed allotypes (reviewed in Jefferis and Lefranc 2009. mAbs Vol 1 Issue 4 1-7) any of which are suitable for use in some of the embodiments herein. Common allotypic variants in human populations are those designated by the letters a, f, n, z or combinations thereof. In any of the embodiments herein, the antibody may comprise a heavy chain Fc region comprising a human IgG Fc region. In further embodiments, the human IgG Fc region comprises a human IgG1.

The antibodies also include derivatives that are modified, i.e., by the covalent attachment of any type of molecule to the antibody such that covalent attachment does not prevent the antibody from binding to PD-1. For example, but not by way of limitation, the antibody derivatives include antibodies that have been modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### D. Nucleic Acids, Host Cells and Methods of Production

In some aspects, also provided herein are nucleic acids encoding an anti-CD30 antibody or antigen-binding fragment thereof as described herein or an anti-PD-1 antibody or antigen-binding fragment thereof as described herein. Further provided herein are vectors comprising the nucleic acids encoding an anti-CD30 antibody or antigen-binding fragment thereof as described herein or an anti-PD-1 antibody or antigen-binding fragment thereof as described herein. Further provided herein are host cells expressing the nucleic acids encoding an anti-CD30 antibody or antigen-binding fragment thereof as described herein or an anti-PD-1 antibody or antigen-binding fragment thereof as described herein. Further provided herein are host cells comprising the vectors comprising the nucleic acids encoding an anti-CD30 antibody or antigen-binding fragment thereof as described herein or an anti-PD-1 antibody or antigen-binding fragment thereof as described herein. Methods of producing an anti-CD30 antibody, linker and anti-CD30 antibody-drug conjugate are described in U.S. Pat. No. 9,211,319.

The anti-CD30 antibodies described herein or anti-PD-1 antibodies described herein may be prepared by well-known recombinant techniques using well known expression vector systems and host cells. In one embodiment, the antibodies are prepared in a CHO cell using the GS expression vector system as disclosed in De la Cruz Edmunds et al., 2006, Molecular Biotechnology 34; 179-190, EP216846, U.S. Pat. No. 5,981,216, WO 87/04462, EP323997, U.S. Pat. Nos. 5,591,639, 5,658,759, EP338841, U.S. Pat. Nos. 5,879,936, and 5,891,693.

After isolating and purifying the anti-CD30 antibodies from the cell media using well known techniques in the art, they are conjugated with monomethyl auristatin E via a linker.

Monoclonal anti-CD30 antibodies described herein or anti-PD-1 antibodies described herein may e.g. be produced by the hybridoma method first described by Kohler et al., Nature, 256, 495 (1975), or may be produced by recombinant DNA methods. Monoclonal antibodies may also be isolated from phage antibody libraries using the techniques described in, for example, Clackson et al., Nature, 352, 624-628 (1991) and Marks et al., J. Mol. Biol., 222 (3): 581-597 (1991). Monoclonal antibodies may be obtained from any suitable source. Thus, for example, monoclonal antibodies may be obtained from hybridomas prepared from murine splenic B cells obtained from mice immunized with an antigen of interest, for instance in form of cells expressing the antigen on the surface, or a nucleic acid encoding an antigen of interest. Monoclonal antibodies may also be obtained from hybridomas derived from antibody-expressing cells of immunized humans or non-human mammals such as rats, dogs, primates, etc.

In one embodiment, the antibody (e.g., anti-CD30 antibody or anti-PD-1 antibody) of the invention is a human antibody. Human monoclonal antibodies directed against CD30 or PD-1 may be generated using transgenic or transchromosomal mice carrying parts of the human immune system rather than the mouse system. Such transgenic and transchromosomic mice include mice referred to herein as HuMAb mice and KM mice, respectively, and are collectively referred to herein as “transgenic mice”.

The HuMAb mouse contains a human immunoglobulin gene minilocus that encodes unrearranged human heavy (μ and γ) and κ light chain immunoglobulin sequences, together with targeted mutations that inactivate the endogenous μ and κ chain loci (Lonberg, N. et al., Nature, 368, 856-859 (1994)). Accordingly, the mice exhibit reduced expression of mouse IgM or κ and in response to immunization, the introduced human heavy and light chain transgenes undergo class switching and somatic mutation to generate high affinity human IgG, κ monoclonal antibodies (Lonberg, N. et al. (1994), supra; reviewed in Lonberg, N. Handbook of Experimental Pharmacology 113, 49-101 (1994), Lonberg, N. and Huszar. D., Intern. Rev. Immunol, Vol. 13 65-93 (1995) and Harding, F. and Lonberg, N. Ann, N.Y. Acad. Sci 764:536-546 (1995)). The preparation of HuMAb mice is described in detail in Taylor, L. et al., Nucleic Acids Research. 20:6287-6295 (1992), Chen, J. et al., International Immunology. 5:647-656 (1993), Tuaillon at al., J. Immunol, 152:2912-2920 (1994), Taylor, L. et al., International Immunology, 6:579-591 (1994), Fishwild, D. et al., Nature Biotechnology, 14:845-851 (1996). See also U.S. Pat. Nos. 5,545,806, 5,569,825, 5,625,126, 5,633,425, 5,789,650, 5,877,397, 5,661,016, 5,814,318, 5,874,299, 5,770,429, 5,545,807, WO 98/24884, WO 94/25585, WO 93/1227, WO 92/22645, WO 92/03918 and WO 01/09187.

The HCo7 mice have a JKD disruption in their endogenous light chain (kappa) genes (as described in Chen et al, EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo7 human heavy chain transgene (as described in U.S. Pat. No. 5,770,429).

The HCo12 mice have a JKD disruption in their endogenous light chain (kappa) genes (as described in Chen et al., EMBO J. 12:821-830 (1993)), a CMD disruption in their endogenous heavy chain genes (as described in Example 1 of WO 01/14424), a KCo5 human kappa light chain transgene (as described in Fishwild et al., Nature Biotechnology, 14:845-851 (1996)), and a HCo12 human heavy chain transgene (as described in Example 2 of WO 01/14424).

The HCo17 transgenic mouse strain (see also US 2010/0077497) was generated by coinjection of the 80 kb insert of pHC2 (Taylor et al. (1994) Int. Immunol., 6:579-591), the Kb insert of pVX6, and a−460 kb yeast artificial chromosome fragment of the yIgH24 chromosome. This line was designated (HCo17) 25950. The (HCo17) 25950 line was then bred with mice comprising the CMD mutation (described in Example 1 of PCT Publication WO 01109187), the JKD mutation (Chen et al, (1993) EMBO J. 12:811-820), and the (KC05) 9272 transgene (Fishwild et al. (1996) Nature Biotechnology, 14:845-851). The resulting mice express human immunoglobulin heavy and kappa light chain trans genes in a background homozygous for disruption of the endogenous mouse heavy and kappa light chain loci.

The HCo20 transgenic mouse strain is the result of a co-injection of minilocus 30 heavy chain transgene pHC2, the germline variable region (Vh)-containing YAC yIgH10, and the minilocus construct pVx6 (described in WO09097006). The (HCo20) line was then bred with mice comprising the CMD mutation (described in Example 1 of PCT Publication WO 01/09187), the JKD mutation (Chen et al. (1993) EMBO J. 12:811-820), and the (KCO5) 9272 trans gene (Fishwild eta). (1996) Nature Biotechnology, 14:845-851). The resulting mice express human 10 immunoglobulin heavy and kappa light chain transgenes in a background homozygous for disruption of the endogenous mouse heavy and kappa light chain loci.

In order to generate HuMab mice with the salutary effects of the Balb/c strain, HuMab mice were crossed with KCO05 [MIK] (Balb) mice which were generated by backcrossing the KC05 strain (as described in Fishwild et (1996) Nature Biotechnology, 14:845-851) to wild-type Balb/c mice to generate mice as described in WO09097006. Using this crossing Balb/c hybrids were created for HCo12, HCo17, and HCo20 strains.

In the KM mouse strain, the endogenous mouse kappa light chain gene has been homozygously disrupted as described in Chen et al., EMBO J. 12:811-820 (1993) and the endogenous mouse heavy chain gene has been homozygously disrupted as described in Example 1 of WO 01/09187, This mouse strain carries a human kappa light chain transgene, KCo5, as described in Fishwild et al., Nature Biotechnology, 14:845-851 (1996). This mouse strain also carries a human heavy chain transchromosome composed of chromosome 14 fragment hCF (SC20) as described in WO 02/43478.

Splenocytes from these transgenic mice may be used to generate hybridomas that secrete human monoclonal antibodies according to well-known techniques. Human monoclonal or polyclonal antibodies of the invention, or antibodies of the invention originating from other species may also be generated transgenically through the generation of another non-human mammal or plant that is transgenic for the immunoglobulin heavy and light chain sequences of interest and production of the antibody in a recoverable form therefrom. In connection with the transgenic production in mammals, antibodies may be produced in, and recovered from, the milk of goats, cows, or other mammals. See for instance U.S. Pat. Nos. 5,827,690, 5,756,687, 5,750,172 and 5,741,957.

Further, human antibodies of the invention or antibodies of the invention from other species may be generated through display-type technologies, including, without limitation, phage display, retroviral display, ribosomal display, and other techniques, using techniques well known in the art and the resulting molecules may be subjected to additional maturation, such as affinity maturation, as such techniques are well known in the art (See for instance Hoogenboom et al., J. Mol, Biol. 227 (2): 381-388 (1992) (phage display), Vaughan et al., Nature Biotech, 14:309 (1996) (phage display), Hanes and Plucthau, PNAS USA 94:4937-4942 (1997) (ribosomal display), Parmley and Smith, Gene, 73:305-318 (1988) (phage display), Scott, TIBS. 17:241-245 (1992), Cwirla et al., PNAS USA, 87:6378-6382 (1990), Russel et al., Nucl. Acids Research, 21:1081-4085 (1993), Hogenboom et al., Immunol, Reviews, 130:43-68 (1992), Chiswell and McCafferty, TIBTECH, 10:80-84 (1992), and U.S. Pat. No. 5,733,743). If display technologies are utilized to produce antibodies that are not human, such antibodies may be humanized.

### III. Binding Assays and Other Assays

In one aspect, an antibody of the invention is tested for its antigen binding activity, for example, by known methods such as Enzyme-Linked Immunosorbant Assay (ELISA), immunoblotting (e.g., Western blotting), flow cytometry (e.g., FACS™), immunohistochemistry, immunofluorescence, etc.

In another aspect, competition assays may be used to identify an antibody that competes with any one of the antibodies described herein for binding to CD30 (e.g., brentuximab) or PD-1 (e.g., pembrolizumab). Cross-competing antibodies can be readily identified based on their ability to cross-compete in standard CD30 or PD-1 binding assays such as Biacore analysis, ELISA assays or flow cytometry (See, e.g., WO 2013/173223). In certain embodiments, such a competing antibody binds to the same epitope (e.g., a linear or a conformational epitope) that is bound by any one of the antibodies disclosed herein (e.g., brentuximab or pembrolizumab). Detailed exemplary methods for mapping an epitope to which an antibody binds are provided in Morris “Epitope Mapping Protocols,” in Methods in Molecular Biology Vol. 66 (Humana Press, Totowa, NJ, 1996).

In an exemplary competition assay, immobilized PD-1 is incubated in a solution comprising a first labeled antibody that binds to PD-1 (e.g., pembrolizumab) and a second unlabeled antibody that is being tested for its ability to compete with the first antibody for binding to PD-1. The second antibody may be present in a hybridoma supernatant. As a control, immobilized PD-1 is incubated in a solution comprising the first labeled antibody but not the second unlabeled antibody. After incubation under conditions permissive for binding of the first antibody to PD-1, excess unbound antibody is removed, and the amount of label associated with immobilized PD-1 is measured. If the amount of label associated with immobilized PD-1 is substantially reduced in the test sample relative to the control sample, then that indicates that the second antibody is competing with the first antibody for binding to PD-1. See, e.g., Harlow et al. Antibodies: A Laboratory Manual. Ch. 14 (Cold Spring Harbor Laboratory, Cold Spring Harbor, NY, 1988). In some embodiments, an anti-PD-1 antibody competes for binding to PD-1 with another PD-1 antibody (e.g., pembrolizumab) if the antibody blocks binding of the other antibody to PD-1 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-PD-1 antibody does not compete for binding to PD-1 with another PD-1 antibody (e.g., pembrolizumab) if the antibody blocks binding of the other antibody to PD-1 in a competition assay by less than 20%, less than 15%, less than 10%, less than 9%, less than 8%, less than 7%, less than 6%, less than 5%, less than 4%, less than 3%, less than 2%, less than 1%. In some embodiments, the PD-1 is human PD-1.

Similar competition assays can be performed to determine if an anti-CD30 antibody competes with brentuximab for binding to CD30. In some embodiments, an anti-CD30 antibody competes for binding to CD30 with another CD30 antibody (e.g., brentuximab) if the antibody blocks binding of the other antibody to CD30 in a competition assay by more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%. In some embodiments, an anti-CD30 antibody does not compete for binding to CD30 with another CD30 antibody (e.g., brentuximab) if the antibody blocks binding of the other antibody to CD30 in a competition assay by less than 20%, less than 15%, less than 10%, less than 9%, less than 8%, less than 7%, less than 6%, less than 5%, less than 4%, less than 3%, less than 2%, less than 1%. In some embodiments, the CD30 is human CD30.

### IV. Methods of Treatment

The invention provides methods for treating cancer in a subject with an anti-CD30 antibody-drug conjugate described herein and an anti-PD-1 antibody described herein, wherein the cancer is a solid tumor. In some embodiments, the solid tumor is selected from the group consisting of non-small cell lung cancer (NSCLC), melanoma, head and neck cancer, renal cell carcinoma, microsatellite instability-high or mismatch repair deficient cancer, bladder cancer, colon cancer, rectal cancer, gastric cancer, gastroesophageal junction carcinoma, esophageal cancer, cervical cancer, hepatocellular carcinoma, endometrial carcinoma, Merkel cell carcinoma, cutaneous squamous cell carcinoma, breast cancer, and tumor mutational burden-high cancer. In one aspect, the antibody-drug conjugate is brentuximab vedotin. In one aspect, the anti-PD-1 antibody is pembrolizumab. In a particular embodiment, the subject is a human.

In another aspect the invention provides an antibody-drug conjugate that binds to CD30 as described herein for use in the treatment of cancer wherein the antibody-drug conjugate is for administration, or to be administered in combination with an anti-PD-1 antibody or an antigen-binding fragment thereof as described herein, wherein the cancer is a solid tumor. In some embodiments, the solid tumor is selected from the group consisting of non-small cell lung cancer (NSCLC), melanoma, head and neck cancer, renal cell carcinoma, microsatellite instability-high or mismatch repair deficient cancer, bladder cancer, colon cancer, rectal cancer, gastric cancer, gastroesophageal junction carcinoma, esophageal cancer, cervical cancer, hepatocellular carcinoma, endometrial carcinoma, Merkel cell carcinoma, cutaneous squamous cell carcinoma, breast cancer, and tumor mutational burden-high cancer. In one aspect, the antibody-drug conjugate is brentuximab vedotin. In one aspect, the anti-PD-1 antibody is pembrolizumab. In a particular embodiment, the subject is a human.

In another aspect the invention provides an anti-PD-1 antibody or an antigen-binding fragment thereof as described herein for use in the treatment of cancer wherein the anti-PD-1 antibody is for administration, or to be administered in combination with an antibody-drug conjugate that binds to CD30, wherein the cancer is a solid tumor. In some embodiments, the solid tumor is selected from the group consisting of non-small cell lung cancer (NSCLC), melanoma, head and neck cancer, renal cell carcinoma, microsatellite instability-high or mismatch repair deficient cancer, bladder cancer, colon cancer, rectal cancer, gastric cancer, gastroesophageal junction carcinoma, esophageal cancer, cervical cancer, hepatocellular carcinoma, endometrial carcinoma, Merkel cell carcinoma, cutaneous squamous cell carcinoma, breast cancer, and tumor mutational burden-high cancer. In one aspect, the antibody-drug conjugate is brentuximab vedotin. In one aspect, the anti-PD-1 antibody is pembrolizumab. In a particular embodiment, the subject is a human.

In some aspects, the invention provides a method of treating cancer in a subject, the method comprising administering to the subject an anti-PD-1 antibody or an antigen-binding fragment thereof and an anti-CD30 antibody-drug conjugate, wherein the cancer is a solid tumor selected from the group consisting of non-small cell lung cancer (NSCLC), melanoma, head and neck cancer, renal cell carcinoma, microsatellite instability-high or mismatch repair deficient cancer, bladder cancer, colon cancer, rectal cancer, gastric cancer, gastroesophageal junction carcinoma, esophageal cancer, cervical cancer, hepatocellular carcinoma, endometrial carcinoma, Merkel cell carcinoma, cutaneous squamous cell carcinoma, breast cancer, and tumor mutational burden-high cancer.

In some embodiments, the cancer is metastatic. In some embodiments, the cancer is non-small lung cancer (NSCLC). In some embodiments, the cancer is squamous NSCLC. In some embodiments, the cancer is nonsquamous NSCLC. In some embodiments, the cancer is primary refractory NSCLC. In some embodiments, the subject progressed without a prior objective response or has stable disease for less than 6 months. In some embodiments, the cancer is relapsed NSCLC. In some embodiments, the subject progressed after having developed a prior objective response of complete response or partial response (CR/PR) for at least 3 months or stable disease for at least 6 months. In some embodiments, the subject does not have a known targetable EGFR, ALK, ROS1, or BRAF mutation. In some embodiments, the subject does not have a known targetable EGFR mutation. In some embodiments, the subject does not have a known targetable ALK mutation. In some embodiments, the subject does not have a known targetable ROS1 mutation. In some embodiments, the subject does not have a known targetable BRAF mutation. In some embodiments, the cancer is melanoma. In some embodiments, the cancer is cutaneous melanoma. In some embodiments, the cancer is primary refractory melanoma. In some embodiments, the subject progressed without a prior objective response or has stable disease for less than 6 months. In some embodiments, the cancer is relapsed melanoma. In some embodiments, the melanoma is unresectable. In some embodiments, the subject progressed after having developed a prior objective response of complete response or partial response (CR/PR) for at least 3 months or stable disease for at least 6 months. In some embodiments, the subject does not have a targetable gene mutation. In some embodiments, the subject is a BRAF-V660E/V600K subject who failed targeted therapy. In some embodiments, the subject is a BRAF-V660E subject who failed targeted therapy. In some embodiments, the subject is a BRAF-V600K subject who failed targeted therapy. In some embodiments, the cancer is head and neck cancer. In some embodiments, the head and neck cancer is squamous cell carcinoma. In some embodiments, the cancer is renal cell carcinoma. In some embodiments, the cancer is microsatellite instability-high or mismatch repair deficient cancer. In some embodiments, the cancer is microsatellite instability-high or mismatch repair deficient colorectal cancer. In some embodiments, the microsatellite instability-high or mismatch repair deficient colorectal cancer is unresectable. In some embodiments, the cancer is bladder cancer. In some embodiments, the bladder cancer is urothelial cancer. In some embodiments, the cancer is high risk, non-muscle invasive bladder cancer. In some embodiments, the cancer is colon cancer. In some embodiments, the cancer is rectal cancer. In some embodiments, the cancer is gastric cancer. In some embodiments, the cancer is gastroesophageal junction carcinoma. In some embodiments, the cancer is gastroesophageal junction adenocarcinoma. In some embodiments, the cancer is esophageal cancer. In some embodiments, the cancer is cervical cancer. In some embodiments, the cancer is hepatocellular carcinoma. In some embodiments, the cancer is endometrial carcinoma. In some embodiments, the endometrial carcinoma is not microsatellite instability-high or mismatch repair deficient. In some embodiments, the cancer is Merkel cell carcinoma. In some embodiments, the cancer is cutaneous squamous cell carcinoma. In some embodiments, the cancer is breast cancer. In some embodiments, the cancer is triple-negative breast cancer. In some embodiments, the cancer is tumor mutational burden-high cancer.

In some embodiments, the subject does not have known active central nervous system metastases and/or carcinomatous meningitis. In some embodiments, the subject does not have known active central nervous system metastases and carcinomatous meningitis. In some embodiments, the subject does not have known active central nervous system metastases or carcinomatous meningitis. In some embodiments, the subject does not have known active central nervous system metastases. In some embodiments, the subject does not have carcinomatous meningitis.

In some embodiments, the cancer cells from the subject do not express CD30. In some embodiments, less than about 0.1%, less than about 0.5%, less than about 1%, less than about 2%, less than about 3%, less than about 4%, less than about 5%, less than about 6%, less than about 7%, less than about 8%, less than about 9%, less than about 10%, less than about 15%, less than about 20%, less than about 25%, or less than about 30% of cancer cells from the subject express CD30. In some embodiments, less than about 0.1% of cancer cells from the subject express CD30. In some embodiments, less than about 0.5% of cancer cells from the subject express CD30. In some embodiments, less than about 1.0% of cancer cells from the subject express CD30. In some embodiments, less than about 2.0% of cancer cells from the subject express CD30. In some embodiments, less than about 3.0% of cancer cells from the subject express CD30. In some embodiments, less than about 4.0% of cancer cells from the subject express CD30. In some embodiments, less than about 5.0% of cancer cells from the subject express CD30. In some embodiments, less than 0.1%, less than 0.5%, less than 1%, less than 2%, less than 3%, less than 4%, less than 5%, less than 6%, less than 7%, less than 8%, less than 9%, less than 10%, less than 15%, less than 20%, less than 25%, or less than 30% of cancer cells from the subject express CD30. In some embodiments, less than 0.1% of cancer cells from the subject express CD30. In some embodiments, less than 0.5% of cancer cells from the subject express CD30. In some embodiments, less than 1.0% of cancer cells from the subject express CD30. In some embodiments, less than 2.0% of cancer cells from the subject express CD30. In some embodiments, less than 3.0% of cancer cells from the subject express CD30. In some embodiments, less than 4.0% of cancer cells from the subject express CD30. In some embodiments, less than 5.0% of cancer cells from the subject express CD30. In some embodiments, the percentage of cancer cells that express CD30 is determined using immunohistochemistry (IHC). In some embodiments, the percentage of cancer cells that express CD30 is determined using flow cytometry. In some embodiments, the percentage of cancer cells that express CD30 is determined using an enzyme-linked immunosorbent assay (ELISA).

In some embodiments, the cancer cells from the subject have been determined to not express CD30. In some embodiments, the cancer cells from the subject have been assessed for CD30 expression. In some embodiments, the cancer cells from the subject have not been assessed for CD30 expression. In some embodiments, the cancer cells from the subject have been screened for CD30 expression and the cancer cells have been determined to not express CD30. In some embodiments, the cancer cells from the subject have not been screened for CD30 expression. In some embodiments, the cancer cells from the subject have been screened for CD30 expression and less than about 0.1%, less than about 0.5%, less than about 1%, less than about 2%, less than about 3%, less than about 4%, or less than about 5% of the cancer cells have been determined to express CD30. In some embodiments, the cancer cells from the subject have been screened for CD30 expression and less than about 0.1% of the cancer cells have been determined to express CD30. In some embodiments, the cancer cells from the subject have been screened for CD30 expression and less than about 0.5% of the cancer cells have been determined to express CD30. In some embodiments, the cancer cells from the subject have been screened for CD30 expression and less than about 1% of the cancer cells have been determined to express CD30. In some embodiments, the cancer cells from the subject have been screened for CD30 expression and less than about 2% of the cancer cells have been determined to express CD30. In some embodiments, the cancer cells from the subject have been screened for CD30 expression and less than about 3% of the cancer cells have been determined to express CD30. In some embodiments, the cancer cells from the subject have been screened for CD30 expression and less than about 4% of the cancer cells have been determined to express CD30. In some embodiments, the cancer cells from the subject have been screened for CD30 expression and less than about 5% of the cancer cells have been determined to express CD30. In some embodiments, the cancer cells from the subject have been screened for CD30 expression and less than 0.1%, less than 0.5%, less than 1%, less than 2%, less than 3%, less than 4%, or less than 5% of the cancer cells have been determined to express CD30. In some embodiments, the cancer cells from the subject have been screened for CD30 expression and less than 0.1% of the cancer cells have been determined to express CD30. In some embodiments, the cancer cells from the subject have been screened for CD30 expression and less than 0.5% of the cancer cells have been determined to express CD30. In some embodiments, the cancer cells from the subject have been screened for CD30 expression and less than 1% of the cancer cells have been determined to express CD30. In some embodiments, the cancer cells from the subject have been screened for CD30 expression and less than 2% of the cancer cells have been determined to express CD30. In some embodiments, the cancer cells from the subject have been screened for CD30 expression and less than 3% of the cancer cells have been determined to express CD30. In some embodiments, the cancer cells from the subject have been screened for CD30 expression and less than 4% of the cancer cells have been determined to express CD30. In some embodiments, the cancer cells from the subject have been screened for CD30 expression and less than 5% of the cancer cells have been determined to express CD30. In some embodiments, the percentage of cancer cells that express CD30 was determined using immunohistochemistry (IHC). In some embodiments, the percentage of cancer cells that express CD30 was determined using flow cytometry. In some embodiments, the percentage of cancer cells that express CD30 was determined using an enzyme-linked immunosorbent assay (ELISA).

In some embodiments, the method further comprises assessing CD30 expression in cancer cells from the subject. In some embodiments, the method further comprises screening the subject for CD30 expression in cancer cells from the subject. In some embodiments, the cancer cells from the subject do not express CD30. In some embodiments, CD30 expression is determined using immunohistochemistry (IHC). In some embodiments, CD30 expression is determined using flow cytometry. In some embodiments, the percentage of cancer cells that express CD30 is determined using an enzyme-linked immunosorbent assay (ELISA).

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of Tregs from the subject express CD30. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of Tregs from the subject express CD30. In some embodiments, the percentage of cells that express CD30 is determined using immunohistochemistry (IHC). In some embodiments, the percentage of cells that express CD30 is determined using flow cytometry. In some embodiments, the percentage of cells that express CD30 is determined using an enzyme-linked immunosorbent assay (ELISA).

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of the cancer cells from the subject express PD-L1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of the cancer cells from the subject express PD-L1. In some of any of the embodiments herein, the subject's tumor expresses PD-L1 with a tumor proportion score (TPS) ≥1%. In some of embodiments herein, the subject's tumor has high PD-L1 expression (TPS≥50%). In some embodiments herein, the subject's tumor expresses PD-L1 with a combined positive score (CPS) ≥1%. See US 2017/0285037. In some embodiments herein, the subject's tumor expresses PD-L1 with a combined positive score (CPS) ≥10%. In some embodiments, the percentage of cells that express PD-L1 is determined using immunohistochemistry (IHC). In some embodiments, the percentage of cells that express PD-L1 is determined using flow cytometry. In some embodiments, the percentage of cells that express PD-L1 is determined using an enzyme-linked immunosorbent assay (ELISA).

In some embodiments, a tumor derived from the cancer comprises one or more cells that express PD-L1, PD-L2, or both PD-L1 and PD-L2.

In some embodiments, at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of T-cells from the subject express PD-1. In some embodiments, at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% of T-cells from the subject express PD-1. In some embodiments, the percentage of cells that express PD-1 is determined using immunohistochemistry (IHC). In some embodiments, the percentage of cells that express PD-1 is determined using flow cytometry. In some embodiments, the percentage of cells that express PD-1 is determined using an enzyme-linked immunosorbent assay (ELISA).

In some embodiments of the methods or uses or product for uses provided herein, the subject has not been previously treated for the cancer. In some embodiments, the subject has been previously treated for the cancer and the subject failed treatment, did not respond to treatment, progressed on treatment, or relapsed after first-line treatment. In some embodiments, the subject failed treatment, did not respond to treatment, progressed on treatment, or relapsed after treatment with an anti-PD-1 monoclonal antibody. In some embodiments, the subject is currently on PD-1 checkpoint inhibitor therapy. In some embodiments, the subject was on a PD-1 checkpoint inhibitor therapy as the last previous line of therapy within 90 days of being administered the combination of the anti-PD-1 antibody or an antigen-binding fragment thereof and the anti-CD30 antibody-drug conjugate. In some embodiments, the subject was previously treated for the cancer with surgery. In some embodiments, the subject was treated with a PD-1 checkpoint inhibitor adjuvant therapy after surgery.

In some embodiments of the methods or uses or product for uses provided herein, the subject has not received prior therapy for the cancer. In some embodiments, the subject has not been previously treated with the anti-CD30 antibody-drug conjugate. In some embodiments, the subject has been previously treated with chemotherapy and/or radiation therapy. In some embodiments, the subject did not respond to the treatment with chemotherapy and radiation therapy. In some embodiments, the subject received treatment for the cancer with chemotherapy and did not respond to the chemotherapy. In some embodiments, the subject received treatment for the cancer with irradiation and did not respond to the irradiation. In some embodiments, the subject relapsed after treatment with chemotherapy and radiation therapy. In some embodiments, the subject received treatment for the cancer with chemotherapy and relapsed after treatment with the chemotherapy. In some embodiments, the subject received treatment for the cancer with irradiation and relapsed after treatment with irradiation. In some embodiments, the subject experienced disease progression after treatment with chemotherapy and/or radiation therapy. In some embodiments, the subject received treatment for the cancer with chemotherapy and experienced disease progression after treatment with the chemotherapy. In some embodiments, the subject received treatment for the cancer with irradiation and experienced disease progression after treatment with irradiation. In some embodiments, the subject has been previously treated for the cancer with one or more therapeutic agents. In some embodiments, the subject has been previously treated with one or more therapeutic agents and did not respond to the treatment. In some embodiments, the subject has been previously treated with one or more therapeutic agents and relapsed after the treatment. In some embodiments, the subject has been previously treated with one or more therapeutic agents and experienced disease progression during treatment. In some embodiments, the subject is not a candidate for curative therapy. In some embodiments, the curative therapy is radiotherapy and/or exenterative therapy. In some embodiments, the curative therapy is radiotherapy. In some embodiments, the curative therapy is exenterative therapy. In a particular embodiment, the subject is a human.

### A. Routes of Administration

An anti-PD-1 antibody or antigen-binding fragment thereof described herein or anti-CD30 antibody-drug conjugate or antigen-binding fragment thereof described herein can be administered by any suitable route and mode. Suitable routes of administering antibodies and/or antibody-drug conjugate of the invention are well known in the art and may be selected by those of ordinary skill in the art. In one embodiment, the anti-PD-1 antibody described herein and/or anti-CD30 antibody-drug conjugate described herein are administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of an anti-CD30 antibody-drug conjugate or antigen-binding fragment described herein is intravenous injection or infusion. In some embodiments, the route of administration of an anti-CD30 antibody-drug conjugate or antigen-binding fragment described herein is intravenous infusion. In some embodiments, the route of administration of an anti-PD-1 antibody or antigen-binding fragment described herein is intravenous injection or infusion. In some embodiments, the route of administration of an anti-PD-1 antibody or antigen-binding fragment described herein is intravenous infusion. In some embodiments, the route of administration of an anti-PD-1 antibody or antigen-binding fragment described herein is subcutaneous.

### B. Dosage and Frequency of Administration

In one aspect, the invention provides for methods of treating a subject with cancer as described herein with a particular dose of an anti-CD30 antibody-drug conjugate or antigen-binding fragment thereof as described herein and an anti-PD-1 antibody or antigen-binding fragment thereof as described herein, wherein the subject is administered the antibody-drug conjugate or antigen-binding fragment thereof as described herein and the anti-PD-1 antibody or antigen-binding fragment thereof as described herein with particular frequencies.

In one embodiment of the methods or uses or product for uses provided herein, an anti-CD30 antibody-drug conjugate or antigen-binding fragment thereof as described herein is administered to the subject at a dose ranging from about 0.6 mg/kg to about 2.3 mg/kg of the subject's body weight. In certain embodiments, the dose is about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1.0 mg/kg, about 1.05 mg/kg, about 1.1 mg/kg, about 1.15 mg/kg, about 1.2 mg/kg, about 1.25 mg/kg, about 1.3 mg/kg, about 1.35 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, or about 2.3 mg/kg. In one embodiment, the dose is about 1.2 mg/kg. In one embodiment, the dose is about 1.8 mg/kg.

In certain embodiments, the dose is 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1.0 mg/kg, 1.05 mg/kg, 1.1 mg/kg, 1.15 mg/kg, 1.2 mg/kg, 1.25 mg/kg, 1.3 mg/kg, 1.35 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, or 2.3 mg/kg. In one embodiment, the dose is 0.65 mg/kg. In one embodiment, the dose is 0.9 mg/kg. In one embodiment, the dose is 1.2 mg/kg. In one embodiment, the dose is 1.8 mg/kg. In some embodiments, the dose is 0.9 mg/kg and the anti-CD30 antibody-drug conjugate is brentuximab vedotin. In some embodiments, the dose is 1.2 mg/kg and the anti-CD30 antibody-drug conjugate is brentuximab vedotin. In some embodiments, the dose is 1.8 mg/kg and the anti-CD30 antibody-drug conjugate is brentuximab vedotin. In some embodiments, for a subject weighing more than 100 kg, the dose of the anti-CD30 antibody-drug conjugate administered is the amount that would be administered if the subject weighed 100 kg. In some embodiments, for a subject weighing more than 100 kg, the dose of the anti-CD30 antibody-drug conjugate administered is 90 mg. In some embodiments, for a subject weighing more than 100 kg, the dose of the anti-CD30 antibody-drug conjugate administered is 120 mg. In some embodiments, for a subject weighing more than 100 kg, the dose of the anti-CD30 antibody-drug conjugate administered is 180 mg.

In one embodiment of the methods or uses or product for uses provided herein, an anti-CD30 antibody-drug conjugate or antigen-binding fragment thereof as described herein is administered to the subject once about every 1 to 4 weeks. In certain embodiments, an anti-CD30 antibody-drug conjugate or antigen-binding fragment thereof as described herein is administered once about every 1 week, once about every 2 weeks, once about every 3 weeks or once about every 4 weeks. In one embodiment, an anti-CD30 antibody-drug conjugate or antigen-binding fragment thereof as described herein is administered once about every 2 weeks. In one embodiment, an anti-CD30 antibody-drug conjugate or antigen-binding fragment thereof as described herein is administered once every 2 weeks. In one embodiment, an anti-CD30 antibody-drug conjugate or antigen-binding fragment thereof as described herein is administered once about every 3 weeks. In one embodiment, an anti-CD30 antibody-drug conjugate or antigen-binding fragment thereof as described herein is administered once every 3 weeks. In some embodiments, the dose is about 0.65 mg/kg and is administered once about every 1 week. In some embodiments, the dose is about 0.65 mg/kg and is administered once about every 2 weeks. In some embodiments, the dose is about 0.65 mg/kg and is administered once about every 3 weeks. In some embodiments, the dose is about 0.65 mg/kg and is administered once about every 4 weeks. In some embodiments, the dose is about 0.7 mg/kg and is administered once about every 1 week. In some embodiments, the dose is about 0.7 mg/kg and is administered once about every 2 weeks. In some embodiments, the dose is about 0.7 mg/kg and is administered once about every 3 weeks. In some embodiments, the dose is about 0.7 mg/kg and is administered once about every 4 weeks. In some embodiments, the dose is about 0.75 mg/kg and is administered once about every 1 week. In some embodiments, the dose is about 0.75 mg/kg and is administered once about every 2 weeks. In some embodiments, the dose is about 0.75 mg/kg and is administered once about every 3 weeks. In some embodiments, the dose is about 0.75 mg/kg and is administered once about every 4 weeks. In some embodiments, the dose is about 0.8 mg/kg and is administered once about every 1 week. In some embodiments, the dose is about 0.8 mg/kg and is administered once about every 2 weeks. In some embodiments, the dose is about 0.8 mg/kg and is administered once about every 3 weeks. In some embodiments, the dose is about 0.8 mg/kg and is administered once about every 4 weeks. In some embodiments, the dose is about 0.85 mg/kg and is administered once about every 1 week. In some embodiments, the dose is about 0.85 mg/kg and is administered once about every 2 weeks. In some embodiments, the dose is about 0.85 mg/kg and is administered once about every 3 weeks. In some embodiments, the dose is about 0.85 mg/kg and is administered once about every 4 weeks. In some embodiments, the dose is about 0.9 mg/kg and is administered once about every 1 week. In some embodiments, the dose is about 0.9 mg/kg and is administered once about every 2 weeks. In some embodiments, the dose is about 0.9 mg/kg and is administered once about every 3 weeks. In some embodiments, the dose is about 0.9 mg/kg and is administered once about every 4 weeks. In some embodiments, the dose is about 0.95 mg/kg and is administered once about every 1 week. In some embodiments, the dose is about 0.95 mg/kg and is administered once about every 2 weeks. In some embodiments, the dose is about 0.95 mg/kg and is administered once about every 3 weeks. In some embodiments, the dose is about 0.95 mg/kg and is administered once about every 4 weeks. In some embodiments, the dose is about 1.0 mg/kg and is administered once about every 1 week. In some embodiments, the dose is about 1.0 mg/kg and is administered once about every 2 weeks. In some embodiments, the dose is about 1.0 mg/kg and is administered once about every 3 weeks. In some embodiments, the dose is about 1.0 mg/kg and is administered once about every 4 weeks. In some embodiments, the dose is about 1.05 mg/kg and is administered once about every 1 week. In some embodiments, the dose is about 1.05 mg/kg and is administered once about every 2 weeks. In some embodiments, the dose is about 1.05 mg/kg and is administered once about every 3 weeks. In some embodiments, the dose is about 1.05 mg/kg and is administered once about every 4 weeks. In some embodiments, the dose is about 1.1 mg/kg and is administered once about every 1 week. In some embodiments, the dose is about 1.1 mg/kg and is administered once about every 2 weeks. In some embodiments, the dose is about 1.1 mg/kg and is administered once about every 3 weeks. In some embodiments, the dose is about 1.1 mg/kg and is administered once about every 4 weeks. In some embodiments, the dose is about 1.15 mg/kg and is administered once about every 1 week. In some embodiments, the dose is about 1.15 mg/kg and is administered once about every 2 weeks. In some embodiments, the dose is about 1.15 mg/kg and is administered once about every 3 weeks. In some embodiments, the dose is about 1.15 mg/kg and is administered once about every 4 weeks. In some embodiments, the dose is about 1.2 mg/kg and is administered once about every 1 week. In some embodiments, the dose is about 1.2 mg/kg and is administered once about every 2 weeks. In some embodiments, the dose is about 1.2 mg/kg and is administered once about every 3 weeks. In some embodiments, the dose is about 1.2 mg/kg and is administered once about every 4 weeks. In some embodiments, the dose is about 1.25 mg/kg and is administered once about every 1 week. In some embodiments, the dose is about 1.25 mg/kg and is administered once about every 2 weeks. In some embodiments, the dose is about 1.25 mg/kg and is administered once about every 3 weeks. In some embodiments, the dose is about 1.25 mg/kg and is administered once about every 4 weeks. In some embodiments, the dose is about 1.3 mg/kg and is administered once about every 1 week. In some embodiments, the dose is about 1.3 mg/kg and is administered once about every 2 weeks. In some embodiments, the dose is about 1.3 mg/kg and is administered once about every 3 weeks. In some embodiments, the dose is about 1.3 mg/kg and is administered once about every 4 weeks. In some embodiments, the dose is about 1.35 mg/kg and is administered once about every 1 week. In some embodiments, the dose is about 1.35 mg/kg and is administered once about every 2 weeks. In some embodiments, the dose is about 1.35 mg/kg and is administered once about every 3 weeks. In some embodiments, the dose is about 1.35 mg/kg and is administered once about every 4 weeks. In some embodiments, the dose is about 1.4 mg/kg and is administered once about every 1 week. In some embodiments, the dose is about 1.4 mg/kg and is administered once about every 2 weeks. In some embodiments, the dose is about 1.4 mg/kg and is administered once about every 3 weeks. In some embodiments, the dose is about 1.4 mg/kg and is administered once about every 4 weeks. In some embodiments, the dose is about 1.5 mg/kg and is administered once about every 1 week. In some embodiments, the dose is about 1.5 mg/kg and is administered once about every 2 weeks. In some embodiments, the dose is about 1.5 mg/kg and is administered once about every 3 weeks. In some embodiments, the dose is about 1.5 mg/kg and is administered once about every 4 weeks. In some embodiments, the dose is about 1.6 mg/kg and is administered once about every 1 week. In some embodiments, the dose is about 1.6 mg/kg and is administered once about every 2 weeks. In some embodiments, the dose is about 1.6 mg/kg and is administered once about every 3 weeks. In some embodiments, the dose is about 1.6 mg/kg and is administered once about every 4 weeks. In some embodiments, the dose is about 1.7 mg/kg and is administered once about every 1 week. In some embodiments, the dose is about 1.7 mg/kg and is administered once about every 2 weeks. In some embodiments, the dose is about 1.7 mg/kg and is administered once about every 3 weeks. In some embodiments, the dose is about 1.7 mg/kg and is administered once about every 4 weeks. In some embodiments, the dose is about 1.8 mg/kg and is administered once about every 1 week. In some embodiments, the dose is about 1.8 mg/kg and is administered once about every 2 weeks. In some embodiments, the dose is about 1.8 mg/kg and is administered once about every 3 weeks. In some embodiments, the dose is about 1.8 mg/kg and is administered once about every 4 weeks. In some embodiments, the dose is about 1.9 mg/kg and is administered once about every 1 week. In some embodiments, the dose is about 1.9 mg/kg and is administered once about every 2 weeks. In some embodiments, the dose is about 1.9 mg/kg and is administered once about every 3 weeks. In some embodiments, the dose is about 1.9 mg/kg and is administered once about every 4 weeks. In some embodiments, the dose is about 2.0 mg/kg and is administered once about every 1 week. In some embodiments, the dose is about 2.0 mg/kg and is administered once about every 2 weeks. In some embodiments, the dose is about 2.0 mg/kg and is administered once about every 3 weeks. In some embodiments, the dose is about 2.0 mg/kg and is administered once about every 4 weeks. In some embodiments, the dose is about 2.1 mg/kg and is administered once about every 1 week. In some embodiments, the dose is about 2.1 mg/kg and is administered once about every 2 weeks. In some embodiments, the dose is about 2.1 mg/kg and is administered once about every 3 weeks. In some embodiments, the dose is about 2.1 mg/kg and is administered once about every 4 weeks. In some embodiments, the dose is about 2.2 mg/kg and is administered once about every 1 week. In some embodiments, the dose is about 2.2 mg/kg and is administered once about every 2 weeks. In some embodiments, the dose is about 2.2 mg/kg and is administered once about every 3 weeks. In some embodiments, the dose is about 2.2 mg/kg and is administered once about every 4 weeks. In some embodiments, the dose is about 2.3 mg/kg and is administered once about every 1 week. In some embodiments, the dose is about 2.3 mg/kg and is administered once about every 2 weeks. In some embodiments, the dose is about 2.3 mg/kg and is administered once about every 3 weeks. In some embodiments, the dose is about 2.3 mg/kg and is administered once about every 4 weeks. In some embodiments, the dose is 0.65 mg/kg and is administered once about every 1 week. In some embodiments, the dose is 0.65 mg/kg and is administered once about every 2 weeks. In some embodiments, the dose is 0.65 mg/kg and is administered once about every 3 weeks. In some embodiments, the dose is 0.65 mg/kg and is administered once about every 4 weeks. In some embodiments, the dose is 0.7 mg/kg and is administered once about every 1 week. In some embodiments, the dose is 0.7 mg/kg and is administered once about every 2 weeks. In some embodiments, the dose is 0.7 mg/kg and is administered once about every 3 weeks. In some embodiments, the dose is 0.7 mg/kg and is administered once about every 4 weeks. In some embodiments, the dose is 0.75 mg/kg and is administered once about every 1 week. In some embodiments, the dose is 0.75 mg/kg and is administered once about every 2 weeks. In some embodiments, the dose is 0.75 mg/kg and is administered once about every 3 weeks. In some embodiments, the dose is 0.75 mg/kg and is administered once about every 4 weeks. In some embodiments, the dose is 0.8 mg/kg and is administered once about every 1 week. In some embodiments, the dose is 0.8 mg/kg and is administered once about every 2 weeks. In some embodiments, the dose is 0.8 mg/kg and is administered once about every 3 weeks. In some embodiments, the dose is 0.8 mg/kg and is administered once about every 4 weeks. In some embodiments, the dose is 0.85 mg/kg and is administered once about every 1 week. In some embodiments, the dose is 0.85 mg/kg and is administered once about every 2 weeks. In some embodiments, the dose is 0.85 mg/kg and is administered once about every 3 weeks. In some embodiments, the dose is 0.85 mg/kg and is administered once about every 4 weeks. In some embodiments, the dose is 0.9 mg/kg and is administered once about every 1 week. In some embodiments, the dose is 0.9 mg/kg and is administered once about every 2 weeks. In some embodiments, the dose is 0.9 mg/kg and is administered once about every 3 weeks. In some embodiments, the dose is 0.9 mg/kg and is administered once about every 4 weeks. In some embodiments, the dose is 0.95 mg/kg and is administered once about every 1 week. In some embodiments, the dose is 0.95 mg/kg and is administered once about every 2 weeks. In some embodiments, the dose is 0.95 mg/kg and is administered once about every 3 weeks. In some embodiments, the dose is 0.95 mg/kg and is administered once about every 4 weeks. In some embodiments, the dose is 1.0 mg/kg and is administered once about every 1 week. In some embodiments, the dose is 1.0 mg/kg and is administered once about every 2 weeks. In some embodiments, the dose is 1.0 mg/kg and is administered once about every 3 weeks. In some embodiments, the dose is 1.0 mg/kg and is administered once about every 4 weeks. In some embodiments, the dose is 1.05 mg/kg and is administered once about every 1 week. In some embodiments, the dose is 1.05 mg/kg and is administered once about every 2 weeks. In some embodiments, the dose is 1.05 mg/kg and is administered once about every 3 weeks. In some embodiments, the dose is 1.05 mg/kg and is administered once about every 4 weeks. In some embodiments, the dose is 1.1 mg/kg and is administered once about every 1 week. In some embodiments, the dose is 1.1 mg/kg and is administered once about every 2 weeks. In some embodiments, the dose is 1.1 mg/kg and is administered once about every 3 weeks. In some embodiments, the dose is 1.1 mg/kg and is administered once about every 4 weeks. In some embodiments, the dose is 1.15 mg/kg and is administered once about every 1 week. In some embodiments, the dose is 1.15 mg/kg and is administered once about every 2 weeks. In some embodiments, the dose is 1.15 mg/kg and is administered once about every 3 weeks. In some embodiments, the dose is 1.15 mg/kg and is administered once about every 4 weeks. In some embodiments, the dose is 1.2 mg/kg and is administered once about every 1 week. In some embodiments, the dose is 1.2 mg/kg and is administered once about every 2 weeks. In some embodiments, the dose is 1.2 mg/kg and is administered once about every 3 weeks. In some embodiments, the dose is 1.2 mg/kg and is administered once about every 4 weeks. In some embodiments, the dose is 1.25 mg/kg and is administered once about every 1 week. In some embodiments, the dose is 1.25 mg/kg and is administered once about every 2 weeks. In some embodiments, the dose is 1.25 mg/kg and is administered once about every 3 weeks. In some embodiments, the dose is 1.25 mg/kg and is administered once about every 4 weeks. In some embodiments, the dose is 1.3 mg/kg and is administered once about every 1 week. In some embodiments, the dose is 1.3 mg/kg and is administered once about every 2 weeks. In some embodiments, the dose is 1.3 mg/kg and is administered once about every 3 weeks. In some embodiments, the dose is 1.3 mg/kg and is administered once about every 4 weeks. In some embodiments, the dose is 1.4 mg/kg and is administered once about every 1 week. In some embodiments, the dose is 1.4 mg/kg and is administered once about every 2 weeks. In some embodiments, the dose is 1.4 mg/kg and is administered once about every 3 weeks. In some embodiments, the dose is 1.4 mg/kg and is administered once about every 4 weeks. In some embodiments, the dose is 1.5 mg/kg and is administered once about every 1 week. In some embodiments, the dose is 1.5 mg/kg and is administered once about every 2 weeks. In some embodiments, the dose is 1.5 mg/kg and is administered once about every 3 weeks. In some embodiments, the dose is 1.5 mg/kg and is administered once about every 4 weeks. In some embodiments, the dose is 1.6 mg/kg and is administered once about every 1 week. In some embodiments, the dose is 1.6 mg/kg and is administered once about every 2 weeks. In some embodiments, the dose is 1.6 mg/kg and is administered once about every 3 weeks. In some embodiments, the dose is 1.6 mg/kg and is administered once about every 4 weeks. In some embodiments, the dose is 1.7 mg/kg and is administered once about every 1 week. In some embodiments, the dose is 1.7 mg/kg and is administered once about every 2 weeks. In some embodiments, the dose is 1.7 mg/kg and is administered once about every 3 weeks. In some embodiments, the dose is 1.7 mg/kg and is administered once about every 4 weeks. In some embodiments, the dose is 1.8 mg/kg and is administered once about every 1 week. In some embodiments, the dose is 1.8 mg/kg and is administered once about every 2 weeks. In some embodiments, the dose is 1.8 mg/kg and is administered once about every 3 weeks. In some embodiments, the dose is 1.8 mg/kg and is administered once about every 4 weeks. In some embodiments, the dose is 1.9 mg/kg and is administered once about every 1 week. In some embodiments, the dose is 1.9 mg/kg and is administered once about every 2 weeks. In some embodiments, the dose is 1.9 mg/kg and is administered once about every 3 weeks. In some embodiments, the dose is 1.9 mg/kg and is administered once about every 4 weeks. In some embodiments, the dose is 2.0 mg/kg and is administered once about every 1 week. In some embodiments, the dose is 2.0 mg/kg and is administered once about every 2 weeks. In some embodiments, the dose is 2.0 mg/kg and is administered once about every 3 weeks. In some embodiments, the dose is 2.0 mg/kg and is administered once about every 4 weeks. In some embodiments, the dose is 2.1 mg/kg and is administered once about every 1 week. In some embodiments, the dose is 2.1 mg/kg and is administered once about every 2 weeks. In some embodiments, the dose is 2.1 mg/kg and is administered once about every 3 weeks. In some embodiments, the dose is 2.1 mg/kg and is administered once about every 4 weeks. In some embodiments, the dose is 2.2 mg/kg and is administered once about every 1 week. In some embodiments, the dose is 2.2 mg/kg and is administered once about every 2 weeks. In some embodiments, the dose is 2.2 mg/kg and is administered once about every 3 weeks. In some embodiments, the dose is 2.2 mg/kg and is administered once about every 4 weeks. In some embodiments, the dose is 2.3 mg/kg and is administered once about every 1 week. In some embodiments, the dose is 2.3 mg/kg and is administered once about every 2 weeks. In some embodiments, the dose is 2.3 mg/kg and is administered once about every 3 weeks. In some embodiments, the dose is 2.3 mg/kg and is administered once about every 4 weeks. In some embodiments, the dose is 1.8 mg/kg and is administered once about every 3 weeks (e.g., +3 days). In some embodiments, the dose is 1.8 mg/kg and is administered once every 3 weeks. In some embodiments, the dose is 1.8 mg/kg and is administered once every 3 weeks and the antibody-drug conjugate is brentuximab vedotin. In some embodiments, the dose is 1.8 mg/kg and is administered on about day 1 of about a 21-day treatment cycle and the antibody-drug conjugate is brentuximab vedotin. The present invention encompasses embodiments wherein the subject remains on the 21-day treatment cycle for at least 2, 3, 4, 5, 6, 7, 8, 9, 10, 11, 12 or more cycles. In another embodiment, the subject remains on the 21-day treatment cycle for between 2 and 48 cycles, such as between 2 and 35 cycles, such as between 2 and 24 cycles, such as between 2 and 15 cycles, such as between 2 and 12 cycles, such as 2 cycles, 3 cycles, 4 cycles, 5 cycles, 6 cycles, 7 cycles, 8 cycles, 9 cycles, 10 cycles, 11 cycles or 12 cycles. In some embodiments, the subject remains on the 21-day treatment cycle for 12 cycles or more, such as 16 cycles or more, such as 24 cycles or more, such as 36 cycles or more. In some embodiments, the 21-day treatment cycle is administered for no more than 3, no more than 4, no more than 5, or no more than 6 three-week treatment cycles. The number of treatment cycles suitable for any specific subject or group of subjects may be determined by a person of skill in the art, typically a physician. In some embodiments, for a subject weighing more than 100 kg, the dose of the anti-CD30 antibody-drug conjugate administered is the amount that would be administered if the subject weighed 100 kg. In some embodiments, for a subject weighing more than 100 kg, the dose of the anti-CD30 antibody-drug conjugate administered is 90 mg. In some embodiments, for a subject weighing more than 100 kg, the dose of the anti-CD30 antibody-drug conjugate administered is 120 mg. In some embodiments, for a subject weighing more than 100 kg, the dose of the anti-CD30 antibody-drug conjugate administered is 180 mg.

In one embodiment of the methods or uses or product for uses provided herein, an anti-PD-1 antibody or antigen-binding fragment thereof as described herein is administered to the subject at flat dose ranging from about 50 mg to about 500 mg such as at a flat dose of about 50 mg or a flat dose of about 60 mg or a flat dose of about 70 mg or a flat dose of about 80 mg or a flat dose of about 90 mg or a flat dose of about 100 mg or a flat dose of about 120 mg or a flat dose of about 140 mg or a flat dose of about 160 mg or a flat dose of about 180 mg or a flat dose of about 200 mg or a flat dose of about 220 mg or a flat dose of about 240 mg or a flat dose of about 260 mg or a flat dose of about 280 mg or a flat dose of about 300 mg or a flat dose of about 320 mg or a flat dose of about 340 mg or a flat dose of about 360 mg or a flat dose of about 380 mg or a flat dose of about 400 mg or a flat dose of about 420 mg or a flat dose of about 440 mg or a flat dose of about 460 mg or a flat dose of about 480 mg or a flat dose of about 500 mg. In some embodiments, the flat dose is about 200 mg. In some embodiments, the flat dose is about 400 mg. In some embodiments of the methods or uses or product for uses provided herein, an anti-PD-1 antibody or antigen-binding fragment thereof as described herein is administered to the subject at flat dose ranging from 50 mg to 500 mg such as at a flat dose of 50 mg or a flat dose of 60 mg or a flat dose of 70 mg or a flat dose of 80 mg or a flat dose of 90 mg or a flat dose of 100 mg or a flat dose of 120 mg or a flat dose of 140 mg or a flat dose of 160 mg or a flat dose of 180 mg or a flat dose of 200 mg or a flat dose of 220 mg or a flat dose of 240 mg or a flat dose of 260 mg or a flat dose of 280 mg or a flat dose of 300 mg or a flat dose of 320 mg or a flat dose of 340 mg or a flat dose of 360 mg or a flat dose of 380 mg or a flat dose of 400 mg or a flat dose of 420 mg or a flat dose of 440 mg or a flat dose of 460 mg or a flat dose of 480 mg or a flat dose of 500 mg. In some embodiments, the flat dose is 200 mg. In some embodiments, the flat dose is 200 mg and the anti-PD-1 antibody is pembrolizumab. In some embodiments, the flat dose is 400 mg. In some embodiments, the flat dose is 400 mg and the anti-PD-1 antibody is pembrolizumab. In some embodiments, the flat dose is about 140 mg and is administered once about every 1 week. In some embodiments, the flat dose is about 140 mg and is administered once about every 2 weeks. In some embodiments, the flat dose is about 140 mg and is administered once about every 3 weeks. In some embodiments, the flat dose is about 140 mg and is administered once about every 4 weeks. In some embodiments, the flat dose is about 160 mg and is administered once about every 1 week. In some embodiments, the flat dose is about 160 mg and is administered once about every 2 weeks. In some embodiments, the flat dose is about 160 mg and is administered once about every 3 weeks. In some embodiments, the flat dose is about 160 mg and is administered once about every 4 weeks. In some embodiments, the flat dose is about 180 mg and is administered once about every 1 week. In some embodiments, the flat dose is about 180 mg and is administered once about every 2 weeks. In some embodiments, the flat dose is about 180 mg and is administered once about every 3 weeks. In some embodiments, the flat dose is about 180 mg and is administered once about every 4 weeks. In some embodiments, the flat dose is about 200 mg and is administered once about every 1 week. In some embodiments, the flat dose is about 200 mg and is administered once about every 2 weeks. In some embodiments, the flat dose is about 200 mg and is administered once about every 3 weeks. In some embodiments, the flat dose is about 200 mg and is administered once about every 4 weeks. In some embodiments, the flat dose is about 220 mg and is administered once about every 1 week. In some embodiments, the flat dose is about 220 mg and is administered once about every 2 weeks. In some embodiments, the flat dose is about 220 mg and is administered once about every 3 weeks. In some embodiments, the flat dose is about 220 mg and is administered once about every 4 weeks. In some embodiments, the flat dose is about 240 mg and is administered once about every 1 week. In some embodiments, the dose is about 240 mg and is administered once about every 2 weeks. In some embodiments, the flat dose is about 240 mg and is administered once about every 3 weeks. In some embodiments, the flat dose is about 240 mg and is administered once about every 4 weeks. In some embodiments, the flat dose is about 260 mg and is administered once about every 1 week. In some embodiments, the flat dose is about 260 mg and is administered once about every 2 weeks. In some embodiments, the flat dose is about 260 mg and is administered once about every 3 weeks. In some embodiments, the flat dose is about 260 mg and is administered once about every 4 weeks. In some embodiments, the flat dose is about 360 mg and is administered once about every 1 week. In some embodiments, the flat dose is about 360 mg and is administered once about every 2 weeks. In some embodiments, the flat dose is about 360 mg and is administered once about every 3 weeks. In some embodiments, the flat dose is about 360 mg and is administered once about every 4 weeks. In some embodiments, the flat dose is about 360 mg and is administered once about every 5 weeks. In some embodiments, the flat dose is about 360 mg and is administered once about every 6 weeks. In some embodiments, the flat dose is about 400 mg and is administered once about every 1 week. In some embodiments, the flat dose is about 400 mg and is administered once about every 2 weeks. In some embodiments, the flat dose is about 400 mg and is administered once about every 3 weeks. In some embodiments, the flat dose is about 400 mg and is administered once about every 4 weeks. In some embodiments, the flat dose is about 400 mg and is administered once about every 5 weeks. In some embodiments, the flat dose is about 400 mg and is administered once about every 6 weeks. In some embodiments, the flat dose is about 440 mg and is administered once about every 1 week. In some embodiments, the flat dose is about 440 mg and is administered once about every 2 weeks. In some embodiments, the flat dose is about 440 mg and is administered once about every 3 weeks. In some embodiments, the flat dose is about 440 mg and is administered once about every 4 weeks. In some embodiments, the flat dose is about 440 mg and is administered once about every 5 weeks. In some embodiments, the flat dose is about 440 mg and is administered once about every 6 weeks. In some embodiments, the flat dose is 140 mg and is administered once about every 1 week. In some embodiments, the flat dose is 140 mg and is administered once about every 2 weeks. In some embodiments, the flat dose is 140 mg and is administered once about every 3 weeks. In some embodiments, the flat dose is 140 mg and is administered once about every 4 weeks. In some embodiments, the flat dose is 160 mg and is administered once about every 1 week. In some embodiments, the flat dose is 160 mg and is administered once about every 2 weeks. In some embodiments, the flat dose is 160 mg and is administered once about every 3 weeks. In some embodiments, the flat dose is 160 mg and is administered once about every 4 weeks. In some embodiments, the flat dose is 180 mg and is administered once about every 1 week. In some embodiments, the flat dose is 180 mg and is administered once about every 2 weeks. In some embodiments, the flat dose is 180 mg and is administered once about every 3 weeks. In some embodiments, the flat dose is 180 mg and is administered once about every 4 weeks. In some embodiments, the flat dose is 200 mg and is administered once about every 1 week. In some embodiments, the flat dose is 200 mg and is administered once about every 2 weeks. In some embodiments, the flat dose is 200 mg and is administered once about every 3 weeks. In some embodiments, the flat dose is 200 mg and is administered once about every 4 weeks. In some embodiments, the flat dose is 220 mg and is administered once about every 1 week. In some embodiments, the flat dose is 220 mg and is administered once about every 2 weeks. In some embodiments, the flat dose is 220 mg and is administered once about every 3 weeks. In some embodiments, the flat dose is 220 mg and is administered once about every 4 weeks. In some embodiments, the flat dose is 240 mg and is administered once about every 1 week. In some embodiments, the flat dose is 240 mg and is administered once about every 2 weeks. In some embodiments, the flat dose is 240 mg and is administered once about every 3 weeks. In some embodiments, the flat dose is 240 mg and is administered once about every 4 weeks. In some embodiments, the flat dose is 260 mg and is administered once about every 1 week. In some embodiments, the flat dose is 260 mg and is administered once about every 2 weeks. In some embodiments, the flat dose is 260 mg and is administered once about every 3 weeks. In some embodiments, the flat dose is 260 mg and is administered once about every 4 weeks. In some embodiments, the flat dose is 360 mg and is administered once about every 1 week. In some embodiments, the flat dose is 360 mg and is administered once about every 2 weeks. In some embodiments, the flat dose is 360 mg and is administered once about every 3 weeks. In some embodiments, the flat dose is 360 mg and is administered once about every 4 weeks. In some embodiments, the flat dose is 360 mg and is administered once about every 5 weeks. In some embodiments, the flat dose is 360 mg and is administered once about every 6 weeks. In some embodiments, the flat dose is 400 mg and is administered once about every 1 week. In some embodiments, the flat dose is 400 mg and is administered once about every 2 weeks. In some embodiments, the flat dose is 400 mg and is administered once about every 3 weeks. In some embodiments, the flat dose is 400 mg and is administered once about every 4 weeks. In some embodiments, the flat dose is 400 mg and is administered once about every 5 weeks. In some embodiments, the flat dose is 400 mg and is administered once about every 6 weeks. In some embodiments, the flat dose is 440 mg and is administered once about every 1 week. In some embodiments, the flat dose is 440 mg and is administered once about every 2 weeks. In some embodiments, the flat dose is 440 mg and is administered once about every 3 weeks. In some embodiments, the flat dose is 440 mg and is administered once about every 4 weeks. In some embodiments, the flat dose is 440 mg and is administered once about every 5 weeks. In some embodiments, the flat dose is 440 mg and is administered once about every 6 weeks. In some embodiments, the flat dose is 200 mg and is administered once about every 3 weeks (e.g., +3 days). In some embodiments, the flat dose is 200 mg and is administered once every 3 weeks. In some embodiments, the flat dose is 200 mg and is administered once every 3 weeks and the antibody is pembrolizumab. In some embodiments, the flat dose is 400 mg and is administered once about every 6 weeks (e.g., +6 days). In some embodiments, the flat dose is 400 mg and is administered once every 6 weeks. In some embodiments, the flat dose is 400 mg and is administered once every 6 weeks and the antibody is pembrolizumab.

In some embodiments of the methods or uses or product for uses provided herein, an anti-PD-1 antibody or antigen-binding fragment thereof as described herein and an anti-CD30 antibody-drug conjugate or antigen-binding fragment thereof as described herein are administered to the subject at a fixed dose. In some embodiments, the fixed dose is based on the amount (e.g., mg) of the antibodies. In certain embodiments, the fixed dose is based on the concentration (e.g., mg/ml) of the antibodies. In some embodiments, the ratio of the amount (e.g., mg) of the anti-PD-1 antibody or antigen-binding fragment thereof as described herein to the amount (e.g., mg) of the anti-CD30 antibody-drug conjugate or antigen-binding fragment thereof as described herein is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1. In some embodiments, the ratio of the amount (e.g., mg) of the anti-PD-1 antibody or antigen-binding fragment thereof as described herein to the amount (e.g., mg) of the anti-CD30 antibody-drug conjugate or antigen-binding fragment thereof as described herein is 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:140, 1:160, 1:180, 1:200, 200:1, 180:1, 160:1, 140:1, 120:1, 100:1, 90:1, 80:1, 70:1, 60:1, 50:1, 40:1, 30:1, 20:1, 15:1, 10:1, 9:1, 8:1, 7:1, 6:1, 5:1, 4:1, 3:1, or 2:1. In some embodiments, the ratio of the concentration (e.g., mg/ml) of the anti-PD-1 antibody or antigen-binding fragment thereof as described herein to the concentration (e.g., mg/ml) of the anti-CD30 antibody-drug conjugate or antigen-binding fragment thereof as described herein is about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1. In some embodiments, the ratio of the concentration (e.g., mg/ml) of the anti-PD-1 antibody or antigen-binding fragment thereof described herein to the concentration (e.g., mg/ml) of the anti-CD30 antibody-drug conjugate or antigen-binding fragment thereof described herein is 1:1, 1:2, 1:3, 1:4, 1:5, 1:6, 1:7, 1:8, 1:9, 1:10, 1:15, 1:20, 1:30, 1:40, 1:50, 1:60, 1:70, 1:80, 1:90, 1:100, 1:120, 1:140, 1:160, 1:180, 1:200, 200:1, 180:1, 160:1, 140:1, 120:1, 100:1, 90:1, 80:1, 70:1, 60:1, 50:1, 40:1, 30:1, 20:1, 15:1, 10:1, 9:1, 8:1, 7:1, 6:1, 5:1, 4:1, 3:1, or 2:1.

In some embodiments, the dose of the anti-CD30 antibody-drug conjugate described herein is 1.8 mg/kg and is administered once about every 3 weeks (e.g., +3 days) and the dose of the anti-PD-1 antibody described herein is 200 mg and is administered once about every 3 weeks (e.g., +3 days). In some embodiments, the dose of the anti-CD30 antibody-drug conjugate described herein is 1.8 mg/kg and is administered once every 3 weeks and the dose of the anti-PD-1 antibody described herein is 200 mg and is administered once every 3 weeks. In some embodiments, the dose of the anti-CD30 antibody-drug conjugate is 1.8 mg/kg and is administered once every 3 weeks and the antibody-drug conjugate is brentuximab vedotin and the dose of the anti-PD-1 antibody is 200 mg and is administered once every 3 weeks and the anti-PD-1 antibody is pembrolizumab.

In some embodiments, an anti-CD30 antibody-drug conjugate or antigen-binding fragment thereof as described herein and an anti-PD-1 antibody or antigen-binding fragment thereof as described herein are coadministered. In some embodiments the coadministration is simultaneous or sequential. In some embodiments, an anti-CD30 antibody-drug conjugate as described herein is administered simultaneously with an anti-PD-1 antibody as described herein. In some embodiments, simultaneous means that the anti-CD30 antibody-drug conjugate as described herein and the anti-PD-1 antibody as described herein are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate as described herein and the anti-PD-1 antibody as described herein are administered to the subject less than one hour apart, such as less than 30 minutes apart, less than 15 minutes apart, less than 10 minutes apart or less than 5 minutes apart. In some embodiments, an anti-CD30 antibody-drug conjugate as described herein is administered sequentially with an anti-PD-1 antibody as described herein. In some embodiments, sequential administration means that the anti-CD30 antibody-drug conjugate as described herein and the anti-PD-1 antibody as described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-PD-1 antibody described herein is administered prior to the anti-CD30 antibody-drug conjugate described herein. In some embodiments, the anti-CD30 antibody-drug conjugate described herein is administered prior to the anti-PD-1 antibody described herein.

In some embodiments, a method of treatment or use described herein further comprises the administration of one or more additional therapeutic agents. In some embodiments, the one or more additional therapeutic agents are administered simultaneously with an anti-CD30 antibody-drug conjugate or antigen-binding fragment thereof as described herein, such as brentuximab vedotin, and an anti-PD-1 antibody or antigen-binding fragment thereof as described herein, such as pembrolizumab. In some embodiments, the one or more additional therapeutic agents and an anti-CD30 antibody-drug conjugate or antigen-binding fragment thereof as described herein and an anti-PD-1 antibody or antigen-binding fragment thereof as described herein are administered sequentially.

### C. Treatment Outcome

In one aspect, a method of treating cancer with an anti-CD30 antibody-drug conjugate or antigen-binding fragment thereof as described herein and an anti-PD-1 antibody or antigen-binding fragment thereof as described herein results in an improvement in one or more therapeutic effects in the subject after administration of the antibody-drug conjugate relative to a baseline. In some embodiments, the one or more therapeutic effects is the size of the tumor derived from the solid tumor, the objective response rate, the duration of response, the time to response, progression free survival, overall survival, or any combination thereof. In one embodiment, the one or more therapeutic effects is the size of the tumor derived from the cancer. In one embodiment, the one or more therapeutic effects is decreased tumor size. In one embodiment, the one or more therapeutic effects is stable disease. In one embodiment, the one or more therapeutic effects is partial response. In one embodiment, the one or more therapeutic effects is complete response. In one embodiment, the one or more therapeutic effects is the objective response rate. In one embodiment, the one or more therapeutic effects is the duration of response. In one embodiment, the one or more therapeutic effects is the time to response. In one embodiment, the one or more therapeutic effects is progression free survival. In one embodiment, the one or more therapeutic effects is overall survival. In one embodiment, the one or more therapeutic effects is cancer regression.

In one embodiment of the methods or uses or product for uses provided herein, response to treatment with an anti-CD30 antibody-drug conjugate or antigen-binding fragment thereof as described herein and an anti-PD-1 antibody or antigen-binding fragment thereof as described herein may include the following criteria (RECIST Criteria 1.1):

| | Category | Criteria |
|---|---|---|
| Based | Complete | Disappearance of all target lesions. Any |
| on | Response | pathological lymph nodes must have reduction in |
| target | (CR) | short axis to <10 mm. |
| lesions | Partial | ≥30% decrease in the sum of the longest diameter |
| | Response | (LD) of target lesions, taking as reference the |
| | (PR) | baseline sum of LDs. |
| | Stable | Neither sufficient shrinkage to qualify for PR nor |
| | Disease | sufficient increase to qualify for PD, taking as |
| | (SD) | reference the smallest sum of LDs while in trial. |
| | Progressive | ≥20% (and ≥5 mm) increase in the sum of the |
| | Disease | LDs of target lesions, taking as reference the |
| | (PD) | smallest sum of the target LDs recorded while |
| | | in trial or the appearance of one or more new |
| | | lesions. |
| Based | CR | Disappearance of all non-target lesions and |
| on non- | | normalization of tumor marker level. All lymph |
| target | | nodes must be non-pathological in size (<10 mm |
| lesions | | short axis). |
| | SD | Persistence of one or more non-target lesion(s) |
| | | or/and maintenance of tumor marker level above |
| | | the normal limits. |
| | PD | Appearance of one or more new lesions and/or |
| | | unequivocal progression of existing non-target |
| | | lesions. |

In one embodiment of the methods or uses or product for uses provided herein, the effectiveness of treatment with an anti-CD30 antibody-drug conjugate or antigen-binding fragment thereof described herein and an anti-PD-1 antibody or antigen-binding fragment thereof described herein is assessed by measuring the objective response rate. In some embodiments, the objective response rate is the proportion of patients with tumor size reduction of a predefined amount and for a minimum period of time. In some embodiments the objective response rate is based upon RECIST v1.1. In one embodiment, the objective response rate is at least about 20%, at least about 25%, at least about 30%, at least about 35%, at least about 40%, at least about 45%, at least about 50%, at least about 60%, at least about 70%, or at least about 80%. In one embodiment, the objective response rate is at least about 20%-80%. In one embodiment, the objective response rate is at least about 30%-80%. In one embodiment, the objective response rate is at least about 40%-80%. In one embodiment, the objective response rate is at least about 50%-80%. In one embodiment, the objective response rate is at least about 60%-80%. In one embodiment, the objective response rate is at least about 70%-80%. In one embodiment, the objective response rate is at least about 80%. In one embodiment, the objective response rate is at least about 85%. In one embodiment, the objective response rate is at least about 90%. In one embodiment, the objective response rate is at least about 95%. In one embodiment, the objective response rate is at least about 98%. In one embodiment, the objective response rate is at least about 99%. In one embodiment, the objective response rate is at least 20%, at least 25%, at least 30%, at least 35%, at least 40%, at least 45%, at least 50%, at least 60%, at least 70%, or at least 80%. In one embodiment, the objective response rate is at least 20%-80%. In one embodiment, the objective response rate is at least 30%-80%. In one embodiment, the objective response rate is at least 40%-80%. In one embodiment, the objective response rate is at least 50%-80%. In one embodiment, the objective response rate is at least 60%-80%. In one embodiment, the objective response rate is at least 70%-80%. In one embodiment, the objective response rate is at least 80%. In one embodiment, the objective response rate is at least 85%. In one embodiment, the objective response rate is at least 90%. In one embodiment, the objective response rate is at least 95%. In one embodiment, the objective response rate is at least 98%. In one embodiment, the objective response rate is at least 99%. In one embodiment, the objective response rate is 100%.

In one embodiment of the methods or uses or product for uses provided herein, response to treatment with an anti-CD30 antibody-drug conjugate or antigen-binding fragment thereof described herein and an anti-PD-1 antibody or antigen-binding fragment thereof described herein is assessed by measuring the size of a tumor derived from the solid tumor. In one embodiment, the size of a tumor derived from the cancer is reduced by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-CD30 antibody-drug conjugate described herein and/or the anti-PD-1 antibody described herein. In one embodiment, the size of a tumor derived from the cancer is reduced by at least about 10%-80%. In one embodiment, the size of a tumor derived from the cancer is reduced by at least about 20%-80%. In one embodiment, the size of a tumor derived from the cancer is reduced by at least about 30%-80%. In one embodiment, the size of a tumor derived from the cancer is reduced by at least about 40%-80%. In one embodiment, the size of a tumor derived from the cancer is reduced by at least about 50%-80%. In one embodiment, the size of a tumor derived from the cancer is reduced by at least about 60%-80%. In one embodiment, the size of a tumor derived from the cancer is reduced by at least about 70%-80%. In one embodiment, the size of a tumor derived from the cancer is reduced by at least about 80%. In one embodiment, the size of a tumor derived from the cancer is reduced by at least about 85%. In one embodiment, the size of a tumor derived from the cancer is reduced by at least about 90%. In one embodiment, the size of a tumor derived from the cancer is reduced by at least about 95%. In one embodiment, the size of a tumor derived from the cancer is reduced by at least about 98%. In one embodiment, the size of a tumor derived from the cancer is reduced by at least about 99%. In one embodiment, the size of a tumor derived from the cancer is reduced by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-CD30 antibody-drug conjugate described herein and/or the anti-PD-1 antibody described herein. In one embodiment, the size of a tumor derived from the cancer is reduced by at least 10%-80%. In one embodiment, the size of a tumor derived from the cancer is reduced by at least 20%-80%. In one embodiment, the size of a tumor derived from the cancer is reduced by at least 30%-80%. In one embodiment, the size of a tumor derived from the cancer is reduced by at least 40%-80%. In one embodiment, the size of a tumor derived from the cancer is reduced by at least 50%-80%. In one embodiment, the size of a tumor derived from the cancer is reduced by at least 60%-80%. In one embodiment, the size of a tumor derived from the cancer is reduced by at least 70%-80%. In one embodiment, the size of a tumor derived from the cancer is reduced by at least 80%. In one embodiment, the size of a tumor derived from the cancer is reduced by at least 85%. In one embodiment, the size of a tumor derived from the cancer is reduced by at least 90%. In one embodiment, the size of a tumor derived from the cancer is reduced by at least 95%. In one embodiment, the size of a tumor derived from the cancer is reduced by at least 98%. In one embodiment, the size of a tumor derived from the cancer is reduced by at least 99%. In one embodiment, the size of a tumor derived from the cancer is reduced by 100%. In one embodiment, the size of a tumor derived from the cancer is measured by magnetic resonance imaging (MRI). In one embodiment, the size of a tumor derived from the cancer is measured by computed tomography (CT). In some embodiments, the size of the tumor derived from the cancer is reduced relative to the size of the tumor before administration of the anti-CD30 antibody drug conjugate described herein and the anti-PD-1 antibody described herein. In some embodiments, the size of the tumor derived from the cancer is reduced relative to the size of the tumor before administration of the anti-CD30 antibody drug conjugate described herein. In some embodiments, the size of the tumor derived from the cancer is reduced relative to the size of the tumor before administration of the anti-PD-1 antibody described herein.

In one embodiment of the methods or uses or product for uses provided described herein, response to treatment with an antibody-drug conjugate or antigen-binding fragment thereof described herein, such as e.g., brentuximab vedotin, and an anti-PD-1 antibody or antigen-binding fragment thereof described herein, such as e.g., pembrolizumab, promotes regression of a tumor derived from the cancer. In one embodiment, a tumor derived from the cancer regresses by at least about 10%, at least about 15%, at least about 20%, at least about 25%, at least about 30%, at least about 35%, at least about 40%, at least about 45%, at least about 50%, at least about 60%, at least about 70%, or at least about 80% relative to the size of the tumor derived from the cancer before administration of the anti-CD30 antibody-drug conjugate described herein and/or anti-PD-1 antibody described herein. In one embodiment, a tumor derived from the cancer regresses by at least about 10% to about 80%. In one embodiment, a tumor derived from the cancer regresses by at least about 20% to about 80%. In one embodiment, a tumor derived from the cancer regresses by at least about 30% to about 80%. In one embodiment, a tumor derived from the cancer regresses by at least about 40% to about 80%. In one embodiment, a tumor derived from the cancer regresses by at least about 50% to about 80%. In one embodiment, a tumor derived from the cancer regresses by at least about 60% to about 80%. In one embodiment, a tumor derived from the cancer regresses by at least about 70% to about 80%. In one embodiment, a tumor derived from the cancer regresses by at least about 80%. In one embodiment, a tumor derived from the cancer regresses by at least about 85%. In one embodiment, a tumor derived from the cancer regresses by at least about 90%. In one embodiment, a tumor derived from the cancer regresses by at least about 95%. In one embodiment, a tumor derived from the cancer regresses by at least about 98%. In one embodiment, a tumor derived from the cancer regresses by at least about 99%. In one embodiment, a tumor derived from the cancer regresses by at least 10%, at least 15%, at least 20%, at least 25%, at least 30%, at least 35%, at least 40%, at least 45%, at least 50%, at least 60%, at least 70%, or at least 80% relative to the size of the tumor derived from the cancer before administration of the anti-CD30 antibody-drug conjugate described herein and/or anti-PD-1 antibody described herein. In one embodiment, a tumor derived from the cancer regresses by at least 10% to 80%. In one embodiment, a tumor derived from the cancer regresses by at least 20% to 80%. In one embodiment, a tumor derived from the cancer regresses by at least 30% to 80%. In one embodiment, a tumor derived from the cancer regresses by at least 40% to 80%. In one embodiment, a tumor derived from the cancer regresses by at least 50% to 80%. In one embodiment, a tumor derived from the cancer regresses by at least 60% to 80%. In one embodiment, a tumor derived from the cancer regresses by at least 70% to 80%. In one embodiment, a tumor derived from the cancer regresses by at least 80%. In one embodiment, a tumor derived from the cancer regresses by at least 85%. In one embodiment, a tumor derived from the cancer regresses by at least 90%. In one embodiment, a tumor derived from the cancer regresses by at least 95%. In one embodiment, a tumor derived from the cancer regresses by at least 98%. In one embodiment, a tumor derived from the cancer regresses by at least 99%. In one embodiment, a tumor derived from the cancer regresses by 100%. In one embodiment, regression of a tumor is determined by measuring the size of the tumor by magnetic resonance imaging (MRI). In one embodiment, regression of a tumor is determined by measuring the size of the tumor by computed tomography (CT). In some embodiments, the tumor derived from the cancer regresses relative to the size of the tumor before administration of the anti-CD30 antibody drug conjugate described herein and the anti-PD-1 antibody described herein. In some embodiments, the tumor derived from the cancer regresses relative to the size of the tumor before administration of the anti-CD30 antibody drug conjugate described herein. In some embodiments, the tumor derived from the cancer regresses relative to the size of the tumor before administration of the anti-PD-1 antibody described herein.

In one embodiment of the methods or uses or product for uses described herein, response to treatment with an anti-CD30 antibody-drug conjugate or antigen-binding fragment thereof described herein and an anti-PD-1 antibody or antigen-binding fragment thereof described herein is assessed by measuring the time of progression free survival after administration of the anti-CD30 antibody-drug conjugate described herein and/or the anti-PD-1 antibody described herein. In some embodiments, the subject exhibits progression-free survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate described herein and/or the anti-PD-1 antibody described herein. In some embodiments, the subject exhibits progression-free survival of at least about 6 months after administration of the anti-CD30 antibody-drug conjugate described herein and/or the anti-PD-1 antibody described herein. In some embodiments, the subject exhibits progression-free survival of at least about one year after administration of the anti-CD30 antibody-drug conjugate described herein and/or the anti-PD-1 antibody described herein. In some embodiments, the subject exhibits progression-free survival of at least about two years after administration of the anti-CD30 antibody-drug conjugate described herein and/or the anti-PD-1 antibody described herein. In some embodiments, the subject exhibits progression-free survival of at least about three years after administration of the anti-CD30 antibody-drug conjugate described herein and/or the anti-PD-1 antibody described herein. In some embodiments, the subject exhibits progression-free survival of at least about four years after administration of the anti-CD30 antibody-drug conjugate described herein and/or the anti-PD-1 antibody described herein. In some embodiments, the subject exhibits progression-free survival of at least about five years after administration of the anti-CD30 antibody-drug conjugate described herein and/or the anti-PD-1 antibody described herein. In some embodiments, the subject exhibits progression-free survival of at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-CD30 antibody-drug conjugate described herein and/or the anti-PD-1 antibody described herein. In some embodiments, the subject exhibits progression-free survival of at least 6 months after administration of the anti-CD30 antibody-drug conjugate described herein and/or the anti-PD-1 antibody described herein. In some embodiments, the subject exhibits progression-free survival of at least one year after administration of the anti-CD30 antibody-drug conjugate described herein and/or the anti-PD-1 antibody described herein. In some embodiments, the subject exhibits progression-free survival of at least two years after administration of the anti-CD30 antibody-drug conjugate described herein and/or the anti-PD-1 antibody described herein. In some embodiments, the subject exhibits progression-free survival of at least three years after administration of the anti-CD30 antibody-drug conjugate described herein and/or the anti-PD-1 antibody described herein. In some embodiments, the subject exhibits progression-free survival of at least four years after administration of the anti-CD30 antibody-drug conjugate described herein and/or the anti-PD-1 antibody described herein. In some embodiments, the subject exhibits progression-free survival of at least five years after administration of the anti-CD30 antibody-drug conjugate described herein and/or the anti-PD-1 antibody described herein. In some embodiments, response to treatment is assessed by measuring the time of progression free survival after administration of the anti-CD30 antibody-drug conjugate described herein and the anti-PD-1 antibody described herein. In some embodiments, response to treatment is assessed by measuring the time of progression free survival after administration of the anti-CD30 antibody-drug conjugate described herein. In some embodiments, response to treatment is assessed by measuring the time of progression free survival after administration of the anti-PD-1 antibody described herein.

In one embodiment of the methods or uses or product for uses described herein, response to treatment with an anti-CD30 antibody-drug conjugate or antigen-binding fragment thereof described herein and an anti-PD-1 antibody or antigen-binding fragment thereof described herein is assessed by measuring the time of overall survival after administration of the anti-CD30 antibody-drug conjugate described herein and/or the anti-PD-1 antibody described herein. In some embodiments, the subject exhibits overall survival of at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate described herein and/or the anti-PD-1 antibody described herein. In some embodiments, the subject exhibits overall survival of at least about 6 months after administration of the anti-CD30 antibody-drug conjugate described herein and/or the anti-PD-1 antibody described herein. In some embodiments, the subject exhibits overall survival of at least about one year after administration of the anti-CD30 antibody-drug conjugate described herein and/or the anti-PD-1 antibody described herein. In some embodiments, the subject exhibits overall survival of at least about two years after administration of the anti-CD30 antibody-drug conjugate described herein and/or the anti-PD-1 antibody described herein. In some embodiments, the subject exhibits overall survival of at least about three years after administration of the anti-CD30 antibody-drug conjugate described herein and/or the anti-PD-1 antibody described herein. In some embodiments, the subject exhibits overall survival of at least about four years after administration of the anti-CD30 antibody-drug conjugate described herein and/or the anti-PD-1 antibody described herein. In some embodiments, the subject exhibits overall survival of at least about five years after administration of the anti-CD30 antibody-drug conjugate described herein and/or the anti-PD-1 antibody described herein. In some embodiments, the subject exhibits overall survival of at least 1 month, at least 2 months, at least 3 months, at least 4 months, at least 5 months, at least 6 months, at least 7 months, at least 8 months, at least 9 months, at least 10 months, at least 11 months, at least about 12 months, at least eighteen months, at least two years, at least three years, at least four years, or at least five years after administration of the anti-CD30 antibody-drug conjugate described herein and/or the anti-PD-1 antibody described herein. In some embodiments, the subject exhibits overall survival of at least 6 months after administration of the anti-CD30 antibody-drug conjugate described herein and/or the anti-PD-1 antibody described herein. In some embodiments, the subject exhibits overall survival of at least one year after administration of the anti-CD30 antibody-drug conjugate described herein and/or the anti-PD-1 antibody described herein. In some embodiments, the subject exhibits overall survival of at least two years after administration of the anti-CD30 antibody-drug conjugate described herein and/or the anti-PD-1 antibody described herein. In some embodiments, the subject exhibits overall survival of at least three years after administration of the anti-CD30 antibody-drug conjugate described herein and/or the anti-PD-1 antibody described herein. In some embodiments, the subject exhibits overall survival of at least four years after administration of the anti-CD30 antibody-drug conjugate described herein and/or the anti-PD-1 antibody described herein. In some embodiments, the subject exhibits overall survival of at least five years after administration of the anti-CD30 antibody-drug conjugate described herein and/or the anti-PD-1 antibody described herein. In some embodiments, response to treatment is assessed by measuring the time of overall survival after administration of the anti-CD30 antibody-drug conjugate described herein and the anti-PD-1 antibody described herein. In some embodiments, response to treatment is assessed by measuring the time of overall survival after administration of the anti-CD30 antibody-drug conjugate described herein. In some embodiments, response to treatment is assessed by measuring the time of overall survival after administration of the anti-PD-1 antibody described herein.

In one embodiment of the methods or uses or product for uses described herein, response to treatment with an anti-CD30 antibody-drug conjugate or antigen-binding fragment thereof described herein and an anti-PD-1 antibody or antigen-binding fragment thereof described herein is assessed by measuring the duration of response to the anti-CD30 antibody-drug conjugate described herein and the anti-PD-1 antibody described herein after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about eighteen months, at least about two years, at least about three years, at least about four years, or at least about five years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least about 6 months after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least about one year after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least about two years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least about three years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least about four years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least about five years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least 1 month, at least 2 months, at least 3 months, at least 4 months, at least 5 months, at least 6 months, at least 7 months, at least 8 months, at least 9 months, at least 10 months, at least 11 months, at least 12 months, at least eighteen months, at least two years, at least three years, at least four years, or at least five years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least 6 months after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least one year after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least two years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least three years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least four years after administration of the anti-CD30 antibody-drug conjugate described herein and/or the anti-PD-1 antibody described herein. In some embodiments, the duration of response to the anti-CD30 antibody-drug conjugate described herein and the anti-PD-1 antibody described herein is at least five years after administration of the anti-CD30 antibody-drug conjugate described herein and/or the anti-PD-1 antibody described herein. In some embodiments, the duration of response is measured after administration of the anti-CD30 antibody drug conjugate described herein and the anti-PD-1 antibody described herein. In some embodiments, the duration of response is measured after administration of the anti-CD30 antibody drug conjugate described herein. In some embodiments, the duration of response is measured after administration of the anti-PD-1 antibody described herein.

In one embodiment of the methods or uses or product for uses described herein, treatment with an anti-CD30 antibody-drug conjugate or antigen-binding fragment thereof described herein decreases the number of CD30+T regulatory cells (Tregs) in the subject. In some embodiments, the number of CD30+ Tregs is decreased by at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90% relative to the number of CD30+ Tregs before administration of an anti-CD30 antibody-drug conjugate or antigen-binding fragment thereof described herein. In some embodiments, the number of CD30+ Tregs is decreased by at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% relative to the number of CD30+ Tregs before administration of an anti-CD30 antibody-drug conjugate or antigen-binding fragment thereof described herein.

In one embodiment of the methods or uses or product for uses described herein, treatment with an anti-PD-1 antibody or antigen-binding fragment thereof described herein results in an upregulation in the amount of CD30 expressed by Tregs by at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% relative to the amount of CD30 expressed by Tregs before administration of the anti-PD-1 antibody or an antigen-binding fragment thereof. In some embodiments, treatment with an anti-PD-1 antibody or antigen-binding fragment thereof described herein results in an upregulation in the amount of CD30 expressed by Tregs by at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, or at least 80% relative to the amount of CD30 expressed by Tregs before administration of the anti-PD-1 antibody or an antigen-binding fragment thereof.

In one embodiment of the methods or uses or product for uses described herein, treatment with an anti-CD30 antibody-drug conjugate or antigen-binding fragment thereof described herein and/or an anti-PD-1 antibody or antigen-binding fragment thereof described herein decreases the number of myeloid-derived suppressor cells (MDSCs) in the subject. In some embodiments, the number of MDSCs is decreased by at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, or at least about 90% relative to the number of MDSCs before administration of an anti-CD30 antibody-drug conjugate or antigen-binding fragment thereof described herein and/or an anti-PD-1 antibody or antigen-binding fragment thereof described herein. In some embodiments, the number of MDSCs is decreased by at least 0.1%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, or at least 90% relative to the number of MDSCs before administration of an anti-CD30 antibody-drug conjugate or antigen-binding fragment thereof described herein and/or an anti-PD-1 antibody or antigen-binding fragment thereof described herein.

In one embodiment of the methods or uses or product for uses described herein, treatment with an anti-CD30 antibody-drug conjugate or antigen-binding fragment thereof described herein and/or an anti-PD-1 antibody or antigen-binding fragment thereof described herein increases the proliferation of T cells in the subject. In some embodiments, the proliferation of T cells is increased by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, or at least about 400% relative to the proliferation of T cells before administration of an anti-CD30 antibody-drug conjugate or antigen-binding fragment thereof described herein and/or an anti-PD-1 antibody or antigen-binding fragment thereof described herein. In some embodiments, the proliferation of T cells is increased by at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, at least 90%, at least 100%, at least 200%, at least 300%, at least 400% relative to the proliferation of T cells before administration of an anti-CD30 antibody-drug conjugate or antigen-binding fragment thereof described herein and/or an anti-PD-1 antibody or antigen-binding fragment thereof described herein.

In one embodiment of the methods or uses or product for uses described herein, treatment with an anti-CD30 antibody-drug conjugate or antigen-binding fragment thereof described herein and/or an anti-PD-1 antibody or antigen-binding fragment thereof described herein increases the infiltration of T cells in the cancer. In some embodiments, the infiltration of T cells is increased by at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 200%, at least about 300%, or at least about 400% relative to the infiltration of T cells before administration of an anti-CD30 antibody-drug conjugate or antigen-binding fragment thereof described herein and/or an anti-PD-1 antibody or antigen-binding fragment thereof described herein. In some embodiments, the infiltration of T cells is increased by at least 5%, at least 6%, at least 7%, at least 8%, at least 9%, at least 10%, at least 15%, at least 20%, at least 25%, at least 30%, at least 35%, at least 40%, at least 45%, at least 50%, at least 60%, at least 70%, at least 80%, at least 90%, at least 100%, at least 200%, at least 300%, at least 400% relative to the infiltration of T cells before administration of an anti-CD30 antibody-drug conjugate or antigen-binding fragment thereof described herein and/or an anti-PD-1 antibody or antigen-binding fragment thereof described herein.

### D. Adverse Events

In one aspect, a method of treating cancer with an anti-CD30 antibody-drug conjugates or antigen-binding fragments thereof described herein and an anti-PD-1 antibody or antigen-binding fragment thereof described herein results in the subject developing one or more adverse events. In some embodiments, the subject is administered an additional therapeutic agent to eliminate or reduce the severity of the adverse event. In some embodiments, the one or more adverse events the subject develops is nausea, fatigue, an infusion related reaction, pruritis, headache, diarrhea, rash, vomiting, cough, dyspnea, pyrexia, nasal congestion, anxiety, myalgia, chills, chest discomfort, dizziness, rash pruritic, alopecia, arthralgia, flushing, peripheral neuropathy, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event.

In one aspect, the subject treated with an anti-CD30 antibody-drug conjugates or antigen-binding fragments thereof described herein and an anti-PD-1 antibody or antigen-binding fragment thereof described herein is at risk of developing one or more adverse events. In some embodiments, the subject is administered an additional therapeutic agent to prevent the development of the adverse event or to reduce the severity of the adverse event. In some embodiments, the one or more adverse events the subject is at risk of developing is nausea, fatigue, an infusion related reaction, pruritis, headache, diarrhea, rash, vomiting, cough, dyspnea, pyrexia, nasal congestion, anxiety, myalgia, chills, chest discomfort, dizziness, rash pruritic, alopecia, arthralgia, flushing, peripheral neuropathy, or any combination thereof. In some embodiments, the one or more adverse events is a grade 1 or greater adverse event. In some embodiments, the one or more adverse events is a grade 2 or greater adverse event. In some embodiments, the one or more adverse events is a grade 3 or greater adverse event. In some embodiments, the one or more adverse events is a grade 1 adverse event. In some embodiments, the one or more adverse events is a grade 2 adverse event. In some embodiments, the one or more adverse events is a grade 3 adverse event. In some embodiments, the one or more adverse events is a grade 4 adverse event. In some embodiments, the one or more adverse events is a serious adverse event.

### V. Compositions

In some aspects, also provided herein are compositions (e.g., pharmaceutical compositions and therapeutic formulations) comprising any of the anti-CD30 antibody-drug conjugates or antigen-binding fragments thereof described herein and/or the anti-PD-1 antibody or antigen-binding fragments thereof described herein.

Therapeutic formulations are prepared for storage by mixing the active ingredient having the desired degree of purity with optional pharmaceutically acceptable carriers, excipients or stabilizers (Remington: The Science and Practice of Pharmacy, 20th Ed., Lippincott Williams & Wiklins, Pub., Gennaro Ed., Philadelphia, Pa. 2000).

Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers, stabilizers, metal complexes (e.g. Zn-protein complexes); chelating agents such as EDTA and/or non-ionic surfactants.

Buffers can be used to control the pH in a range which optimizes the therapeutic effectiveness, especially if stability is pH dependent. Buffers can be present at concentrations ranging from about 50 mM to about 250 mM. Suitable buffering agents for use with the invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives can be added to prevent microbial growth, and are typically present in a range from about 0.2%-1.0% (w/v). Suitable preservatives for use with the invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” can be present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter and intramolecular interactions. Tonicity agents can be present in any amount between about 0.1% to about 25% by weight or between about 1% to about 5% by weight, taking into account the relative amounts of the other ingredients. In some embodiments, tonicity agents include polyhydric sugar alcohols, trihydric or higher sugar alcohols, such as glycerin, erythritol, arabitol, xylitol, sorbitol and mannitol.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, a-monothioglycerol and sodium thio sulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

Non-ionic surfactants or detergents (also known as “wetting agents”) can be present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml or about 0.07 mg/ml to about 0.2 mg/ml. In some embodiments, non-ionic surfactants are present in a range of about 0.001% to about 0.1% w/v or about 0.01% to about 0.1% w/v or about 0.01% to about 0.025% w/v.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl celluose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

In some embodiments provided herein, a formulation comprising the anti-CD30 antibody-conjugate described herein or anti-PD-1 antibody described herein does not comprise a surfactant (i.e., is free of surfactant).

In order for the formulations to be used for in vivo administration, they must be sterile. The formulation may be rendered sterile by filtration through sterile filtration membranes. The therapeutic compositions herein generally are placed into a container having a sterile access port, for example, an intravenous solution bag or vial having a stopper pierceable by a hypodermic injection needle.

The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The invention provides compositions comprising a population of anti-CD30 antibody-drug conjugates or antigen-binding fragments thereof as described herein for use in a method of treating a solid tumor as described herein. In some aspects, provided herein are compositions comprising a population of antibody-drug conjugates, wherein the antibody-drug conjugates comprise a linker attached to MMAE, wherein the antibody-drug conjugate has the following structure:

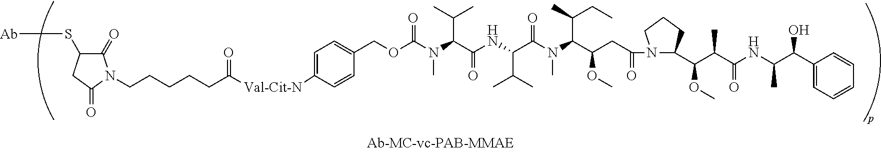

wherein p denotes a number from 1 to 8, e.g., 1, 2, 3, 4, 5, 6, 7 or 8, S represents a sulphydryl residue of the anti-CD30 antibody or antigen-binding fragment thereof, and Ab designates the anti-CD30 antibody or antigen-binding fragment thereof as described herein, such as brentuximab. In some embodiments, p denotes a number from 3 to 5. In some embodiments, the average value of p in the composition is about 4. In some embodiments, the population is a mixed population of anti-CD30 antibody-drug conjugates in which p varies from 1 to 8 for each anti-CD30 antibody-drug conjugate. In some embodiments, the population is a homogenous population of anti-CD30 antibody-drug conjugates with each anti-CD30 antibody-drug conjugate having the same value for p.

In some embodiments, a composition comprising an anti-CD30 antibody-drug conjugate or antigen-binding fragment thereof as described herein is coadministered with a composition comprising an anti-PD-1 antibody or antigen-binding fragment thereof as described herein. In some embodiments the coadministration is simultaneous or sequential. In some embodiments, the anti-CD30 antibody-drug conjugate as described herein is administered simultaneously with the anti-PD-1 antibody as described herein. In some embodiments, simultaneous means that the anti-CD30 antibody-drug conjugate described herein and the anti-PD-1 antibody described herein are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and the anti-PD-1 antibody described herein are administered to the subject less than one hour apart, such as less than 30 minutes apart, less than 15 minutes apart, less than 10 minutes apart or less than 5 minutes apart. In some embodiments, the anti-CD30 antibody-drug conjugate described herein is administered sequentially with the anti-PD-1 antibody described herein. In some embodiments, sequential administration means that the anti-CD30 antibody-drug conjugate described herein and the anti-PD-1 antibody described herein are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-PD-1 antibody described herein is administered prior to the anti-CD30 antibody-drug conjugate described herein. In some embodiments, the anti-CD30 antibody-drug conjugate described herein is administered prior to the anti-PD-1 antibody described herein.

In some embodiments, a composition comprising an anti-CD30 antibody-drug conjugate as described herein and/or anti-PD-1 antibody as described herein is coadministered with one or additional therapeutic agents. In some embodiments the coadministration is simultaneous or sequential. In some embodiments, the anti-CD30 antibody-drug conjugate as described herein and/or anti-PD-1 antibody as described herein is administered simultaneously with the one or more additional therapeutic agents. In some embodiments, simultaneous means that the anti-CD30 antibody-drug conjugate described herein and/or anti-PD-1 antibody described herein and the one or more therapeutic agents are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and/or anti-PD-1 antibody described herein and the one or more therapeutic agents are administered to the subject less than one hour apart, such as less than 30 minutes apart, less than 15 minutes apart, less than 10 minutes apart or less than 5 minutes apart. In some embodiments, the anti-CD30 antibody-drug conjugate described herein and/or anti-PD-1 antibody described herein is administered sequentially with the one or more additional therapeutic agents. In some embodiments, sequential administration means that the anti-CD30 antibody-drug conjugate described herein and/or anti-PD-1 antibody described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart.

In some embodiments, a composition comprising an anti-CD30 antibody-drug conjugate as described herein and/or anti-PD-1 antibody as described herein is coadministered with one or more therapeutic agents to eliminate or reduce the severity of one or more adverse events. In some embodiments the coadministration is simultaneous or sequential. In some embodiments, the anti-CD30 antibody-drug conjugate described herein and/or anti-PD-1 antibody described herein is administered simultaneously with the one or more therapeutic agents to eliminate or reduce the severity of one or more adverse events. In some embodiments, simultaneous means that the anti-CD30 antibody-drug conjugate described herein and/or anti-PD-1 antibody described herein and the one or more therapeutic agents to eliminate or reduce the severity of one or more adverse events are administered to the subject less than about one hour apart, such as less than about 30 minutes apart, less than about 15 minutes apart, less than about 10 minutes apart or less than about 5 minutes apart. In some embodiments, simultaneous means that the anti-CD30 antibody-drug conjugate described herein and/or anti-PD-1 antibody described herein and the one or more therapeutic agents to eliminate or reduce the severity of one or more adverse events are administered to the subject less than one hour apart, such as less than 30 minutes apart, less than 15 minutes apart, less than 10 minutes apart or less than 5 minutes apart. In some embodiments, the anti-CD30 antibody-drug conjugate described herein and/or anti-PD-1 antibody described herein is administered sequentially with the one or more therapeutic agents to eliminate or reduce the severity of one or more adverse events. In some embodiments, sequential administration means that the anti-CD30 antibody-drug conjugate described herein and/or anti-PD-1 antibody described herein and the one or more additional therapeutic agents are administered a least 1 hour apart, at least 2 hours apart, at least 3 hours apart, at least 4 hours apart, at least 5 hours apart, at least 6 hours apart, at least 7 hours apart, at least 8 hours apart, at least 9 hours apart, at least 10 hours apart, at least 11 hours apart, at least 12 hours apart, at least 13 hours apart, at least 14 hours apart, at least 15 hours apart, at least 16 hours apart, at least 17 hours apart, at least 18 hours apart, at least 19 hours apart, at least 20 hours apart, at least 21 hours apart, at least 22 hours apart, at least 23 hours apart, at least 24 hours apart, at least 2 days apart, at least 3 days apart, at least 4 days apart, at least 5 days apart, at least 5 days apart, at least 7 days apart, at least 2 weeks apart, at least 3 weeks apart or at least 4 weeks apart. In some embodiments, the anti-CD30 antibody-drug conjugate described herein and/or anti-PD-1 antibody described herein is administered prior to the one or more therapeutic agents to eliminate or reduce the severity of one or more adverse events. In some embodiments, the one or more therapeutic agents to eliminate or reduce the severity of one or more adverse events is administered prior to the anti-CD30 antibody-drug conjugate described herein and/or anti-PD-1 antibody described herein.

### VI. Articles of Manufacture and Kits

In another aspect, an article of manufacture or kit is provided which comprises an anti-CD30 antibody-drug conjugate described herein and/or an anti-PD-1 antibody described herein. The article of manufacture or kit may further comprise instructions for use of the anti-CD30 antibody-drug conjugate described herein and/or anti-PD-1 antibody described herein in the methods of the invention. Thus, in certain embodiments, the article of manufacture or kit comprises instructions for the use of an anti-CD30 antibody-drug conjugate described herein and/or an anti-PD-1 antibody described herein in methods for treating cancer in a subject comprising administering to the subject an effective amount of an anti-CD30 antibody-drug conjugate described herein and/or anti-PD-1 antibody described herein, wherein the cancer is a solid tumor. In some embodiments, the cancer is metastatic. In some embodiments, the cancer is non-small lung cancer (NSCLC). In some embodiments, the cancer is squamous NSCLC. In some embodiments, the cancer is nonsquamous NSCLC. In some embodiments, the cancer is primary refractory NSCLC. In some embodiments, the subject progressed without a prior objective response or has stable disease for less than 6 months. In some embodiments, the cancer is relapsed NSCLC. In some embodiments, the subject progressed after having developed a prior objective response of complete response or partial response (CR/PR) for at least 3 months or stable disease for at least 6 months. In some embodiments, the subject does not have a known targetable EGFR, ALK, ROS1, or BRAF mutation. In some embodiments, the subject does not have a known targetable EGFR mutation. In some embodiments, the subject does not have a known targetable ALK mutation. In some embodiments, the subject does not have a known targetable ROS1 mutation. In some embodiments, the subject does not have a known targetable BRAF mutation. In some embodiments, the cancer is melanoma. In some embodiments, the cancer is cutaneous melanoma. In some embodiments, the cancer is primary refractory melanoma. In some embodiments, the subject progressed without a prior objective response or has stable disease for less than 6 months. In some embodiments, the cancer is relapsed melanoma. In some embodiments, the melanoma is unresectable. In some embodiments, the subject progressed after having developed a prior objective response of complete response or partial response (CR/PR) for at least 3 months or stable disease for at least 6 months. In some embodiments, the subject does not have a targetable gene mutation. In some embodiments, the subject is a BRAF-V660E/V600K subject who failed targeted therapy. In some embodiments, the subject is a BRAF-V660E subject who failed targeted therapy. In some embodiments, the subject is a BRAF-V600K subject who failed targeted therapy. In some embodiments, the cancer is head and neck cancer. In some embodiments, the head and neck cancer is squamous cell carcinoma. In some embodiments, the cancer is renal cell carcinoma. In some embodiments, the cancer is microsatellite instability-high or mismatch repair deficient cancer. In some embodiments, the cancer is microsatellite instability-high or mismatch repair deficient colorectal cancer. In some embodiments, the microsatellite instability-high or mismatch repair deficient colorectal cancer is unresectable. In some embodiments, the cancer is bladder cancer. In some embodiments, the bladder cancer is urothelial cancer. In some embodiments, the cancer is high risk, non-muscle invasive bladder cancer. In some embodiments, the cancer is colon cancer. In some embodiments, the cancer is rectal cancer. In some embodiments, the cancer is gastric cancer. In some embodiments, the cancer is gastroesophageal junction carcinoma. In some embodiments, the cancer is gastroesophageal junction adenocarcinoma. In some embodiments, the cancer is esophageal cancer. In some embodiments, the cancer is cervical cancer. In some embodiments, the cancer is hepatocellular carcinoma. In some embodiments, the cancer is endometrial carcinoma. In some embodiments, the endometrial carcinoma is not microsatellite instability-high or mismatch repair deficient. In some embodiments, the cancer is Merkel cell carcinoma. In some embodiments, the cancer is cutaneous squamous cell carcinoma. In some embodiments, the cancer is breast cancer. In some embodiments, the cancer is triple-negative breast cancer. In some embodiments, the cancer is tumor mutational burden-high cancer. In some embodiments, the subject is a human.

The article of manufacture or kit may further comprise a container. Suitable containers include, for example, bottles, vials (e.g., dual chamber vials), syringes (such as single or dual chamber syringes) and test tubes. In some embodiments, the container is a vial. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for subcutaneous, intravenous (e.g., intravenous infusion), or other modes of administration for treating a solid tumor in a subject described herein. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

The article of manufacture or kit herein optionally further comprises a container comprising a second medicament, wherein the anti-CD30 antibody-drug conjugate described herein is a first medicament, and which article or kit further comprises instructions on the label or package insert for treating the subject with the second medicament, in an effective amount. In some embodiments, the second medicament is an anti-PD-1 antibody as described herein. In some embodiments, the label or package insert indicates that the first and second medicaments are to be administered sequentially or simultaneously, as described herein.

The article of manufacture or kit herein optionally further comprises a container comprising a third medicament, wherein the third medicament is for eliminating or reducing the severity of one or more adverse events, wherein the anti-CD30 antibody-drug conjugate described herein is a first medicament, the anti-PD-1 antibody described herein is a second medicament, and which article or kit further comprises instructions on the label or package insert for treating the subject with the third medicament, in an effective amount. In some embodiments, the label or package insert indicates that the first, second and third medicaments are to be administered sequentially or simultaneously, as described herein, for example wherein the label or package insert indicates that the anti-CD30 antibody-drug conjugate described herein is to be administered first, followed by administration of the anti-PD-1 antibody described herein, followed by administration of the third medicament.

In some embodiments, the anti-CD30 antibody-drug conjugate described herein and/or anti-PD-1 antibody described herein is present in the container as a lyophilized powder. In some embodiments, the lyophilized powder is in a hermetically sealed container, such as a vial, an ampoule or sachette, indicating the quantity of the active agent. Where the pharmaceutical is administered by injection, an ampoule of sterile water for injection or saline can be, for example, provided, optionally as part of the kit, so that the ingredients can be mixed prior to administration. Such kits can further include, if desired, one or more of various conventional pharmaceutical components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components can also be included in the kit.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: A Phase II Trial of Brentuximab Vedotin in Combination with a Pembrolizumab in Subjects with Metastatic Solid Malignancies

This is an ongoing open-label, multi-cohort, multicenter, phase 2 clinical trial designed to evaluate the safety and antitumor activity of brentuximab vedotin in combination with pembrolizumab for the treatment of subjects with metastatic NSCLC (without known targetable EGFR, ALK, ROS1, or BRAF mutations) or metastatic cutaneous melanoma (including subjects without targetable gene mutations and BRAF-V600E/V600K subjects who have failed targeted therapy) who have progressed on or following treatment with PD-1 inhibitor therapy as last previous line of therapy.

Subjects are being divided into 4 cohorts of approximately 15 subjects per cohort based on indication (either NSCLC or melanoma) and subjects whose disease is relapsed/refractory to prior PD-1 treatment. All subjects are being treated with brentuximab vedotin 1.8 mg/kg via intravenous infusion every 3 weeks in combination with pembrolizumab 200 mg every 3 weeks for a maximum of 35 cycles. 15/15 subjects with relapsed melanoma and 9/15 subjects with relapsed NSCLC have been enrolled and administered brentuximab vedotin and pembrolizumab as described in this example. 4/15 subjects with primary refractory melanoma and 5/15 subjects with primary refractory NSCLC have been enrolled and administered brentuximab vedotin and pembrolizumab as described in this example.

Brentuximab vedotin 1.8 mg/kg is being administered on Day 1 of every 21-day cycle by IV infusion given over approximately 30 minutes. In the absence of infusion-related reactions, the infusion rate for all subjects should be calculated in order to achieve a 30-minute infusion period. Investigational agent must not be administered as an IV push or bolus. Investigational agent should not be mixed with other medications. Administration of brentuximab vedotin begins at least 30 minutes after completion of infusion of pembrolizumab.

Weight-based dosing is based on subject actual body weight. Doses must be adjusted for subjects who experience a ≥10% change in weight from baseline. Subject weight must be measured during all relevant assessment windows as described in the schedule of events. Other dose adjustments for changes in body weight are permitted per institutional standard. Rounding is permissible within 5% of the nominal dose. An exception to weight-based dosing is made for subjects weighing greater than 100 kg; doses will be based on 100 kg for these individuals. The brentuximab vedotin maximum dose calculated per cycle in this study is 180 mg.

Table 1 describes the recommended dose modifications for study treatment associated-toxicity for all subjects. Pembrolizumab dosing may continue if brentuximab vedotin dosing is modified or stopped if the subject is deriving clinical benefit. However, subjects who discontinue pembrolizumab will also discontinue brentuximab vedotin.

Doses reduced for treatment-related toxicity should not be re-escalated without discussion with the sponsor. Please refer to the brentuximab vedotin package insert for detailed recommendation on dose delay/modification.

TABLE 1
Recommended dose modifications for brentuximab vedotin-associated toxicity

| Toxicity | Grade 1 | Grade 2 | Grade 3 | Grade 4 |
|---|---|---|---|---|
| Peripheral | Continue at | Withhold until toxicity resolves to Grade 1 | | Discontinue treatment |
| Neuropathy | same dose level | or baseline, then resume treatment at | | |
| | | 1.2 mg/kg^{a} | | |
| Non-hematologic | Continue at | Continue at same | Withhold dose until | Withhold dose until |
| (except peripheral | same dose level | dose level | toxicity is Grade ≤ 2 or | toxicity is Grade ≤ 2 |
| neuropathy) | | | has returned to | or has returned to |
| | | | baseline, then resume | baseline, then reduce |
| | | | treatment at the same | dose to 1.2 mg/kg and |
| | | | dose level^{b}. | resume treatment, or |
| | | | | discontinue at the |
| | | | | discretion of the |
| | | | | investigator^{a, b, c}. |

| | | | |
|---|---|---|---|
| Hematologic^{d} | Continue at | Continue at same | Withhold until toxicity resolves to Grade ≤ 2 or |
| | same dose level | dose level | baseline, then resume treatment at the same dose |
| | | | level^{e}. For Grade 3 or 4 neutropenia, growth |
| | | | factor support (G-CSF or GM-CSF) should be |
| | | | considered for subsequent cycles. If Grade 4 |
| | | | neutropenia recurs despite growth factor |
| | | | support, consider discontinuation or dose |
| | | | reduction to 1.2 mg/kg. |

^{a}Dose reductions below 1.2 mg/kg are not allowed, and toxicities should be managed with dose delays.
^{b}Subjects who develop Grade 3 or 4 electrolyte laboratory abnormalities may continue study treatment without interruption.
^{c}Treatment should be discontinued for subjects who experience Grade 4 infusion-related reactions.
^{d}Support with blood product transfusions allowed per institutional standard of care.
^{e}Subjects who develop Grade 3 or 4 lymphopenia may continue study treatment without interruption.

Study treatment of pembrolizumab is being administered on Day 1 of each 21-day cycle. Pembrolizumab will be administered as a dose of 200 mg using a 30-minute IV infusion; administration of pembrolizumab should be completed at least 30 minutes prior to infusion of brentuximab vedotin. Sites should make every effort to target infusion timing to be as close to 30 minutes as possible. However, given the variability of infusion pumps from site to site, a window between-5 minutes and +10 minutes is permitted (i.e., infusion time is 30 minutes-5 min/+10 min).

Brentuximab vedotin dosing should be discontinued if pembrolizumab dosing is discontinued. However, subjects who discontinue brentuximab vedotin may continue to receive pembrolizumab if the subject is deriving clinical benefit.

Dose modification and toxicity management guidelines for irAEs associated with pembrolizumab are provided in Table 2.

TABLE 2
Dose modification and toxicity management guidelines for immune-
related AEs associated with pembrolizumab
General instructions:
1. Severe and life-threatening irAEs should be treated with IV corticosteroids followed by oral
steroids. Other immunosuppressive treatment should begin if the irAEs are not controlled by
corticosteroids.
2. Pembrolizumab must be permanently discontinued if the irAE does not resolve or the
corticosteroid dose is not ≤10 mg/day within 12 weeks of the last pembrolizumab treatment.
3. The corticosteroid taper should begin when the irAE is ≤Grade 1 and continue at least 4 weeks.
4. If pembrolizumab has been withheld, pembrolizumab may resume after the irAE decreased
to ≤Grade 1 after corticosteroid taper.

| | Toxicity grade | Action with | Corticosteroid and/or | Monitoring and |
|---|---|---|---|---|
| irAEs | (CTCAE V5.0) | pembrolizumab | other therapies | follow-up |
| Pneumonitis | Grade 2 | Withhold | Administer | Monitor participants for |
| | Recurrent | Permanently | corticosteroids | signs and |
| | Grade 2, | discontinue | (initial dose of | symptoms of |
| | Grade 3 or 4 | | 1 to 2 mg/kg | pneumonitis |
| | | | prednisone or | Evaluate participants |
| | | | equivalent) | with suspected |
| | | | followed by taper | pneumonitis with |
| | | | Add prophylactic | radiographic |
| | | | antibiotics for | imaging and |
| | | | opportunistic | initiate |
| | | | infections | corticosteroid |
| | | | | treatment |
| Diarrhea/ | Grade 2 or 3 | Withhold | Administer | Monitor participants for |
| Colitis | Recurrent | Permanently | corticosteroids | signs and |
| | Grade 3 or | discontinue | (initial dose of | symptoms of |
| | Grade 4 | | 1 to 2 mg/kg | enterocolitis (ie, |
| | | | prednisone or | diarrhea, |
| | | | equivalent) | abdominal pain, |
| | | | followed by taper | blood or mucus in |
| | | | | stool with or |
| | | | | without fever) and |
| | | | | of bowel |
| | | | | perforation (ie. |
| | | | | peritoneal signs |
| | | | | and ileus) |
| | | | | Participants with |
| | | | | ≥Grade 2 diarrhea |
| | | | | suspecting colitis |
| | | | | should consider GI |
| | | | | consultation and |
| | | | | performing |
| | | | | endoscopy to rule |
| | | | | out colitis |
| | | | | Participants with |
| | | | | diarrhea/colitis |
| | | | | should be advised |
| | | | | to drink liberal |
| | | | | quantities of clear |
| | | | | fluids. If sufficient |
| | | | | oral fluid intake is |
| | | | | not feasible, fluid |
| | | | | and electrolytes |
| | | | | should be |
| | | | | substituted via IV |
| | | | | infusion |
| AST or ALT | Grade 2 ^{a} | Withhold | Administer | Monitor with liver |
| elevation or | | | corticosteroids | function tests |
| Increased | | | (initial dose of | (consider weekly or |
| Bilirubin | | | 0.5 to 1 mg/kg | more frequently |
| | | | prednisone or | until liver enzyme |
| | | | equivalent) | value returned to |
| | | | followed by taper | baseline or is |
| | Grade 3 ^{b} or 4 ^{c} | Permanently | Administer | stable) |
| | | discontinue | corticosteroids | |
| | | | (initial dose of | |
| | | | 1 to 2 mg/kg | |
| | | | prednisone or | |
| | | | equivalent) | |
| | | | followed by taper | |
| Type 1 diabetes | New onset | Withhold ^{d} | Initiate insulin | Monitor participants for |
| mellitus | T1DM or | | replacement | hyperglycemia or |
| (T1DM) or | Grade 3 or 4 | | therapy for | other signs and |
| Hyperglycemia | hyperglycemia | | participants with | symptoms of |
| | associated with | | T1DM | diabetes |
| | evidence of | | Administer | |
| | β-cell failure | | antihyperglycemic | |
| | | | in participants with | |
| | | | hyperglycemia | |
| Hypophysitis | Grade 2 | Withhold | Administer | Monitor for signs and |
| | Grade 3 or 4 | Withhold or | corticosteroids and | symptoms of |
| | | permanently | initiate hormonal | hypophysitis |
| | | discontinue ^{d} | replacements as | (including |
| | | | clinically indicated | hypopituitarism |
| | | | | and adrenal |
| | | | | insufficiency) |
| Hyperthyroidism | Grade 2 | Continue | Treat with nonselective | Monitor for signs and |
| | Grade 3 or 4 | Withhold or | beta-blockers (eg, | symptoms of |
| | | permanently | propranolol) or | thyroid disorders |
| | | discontinue ^{d} | thionamides as | |
| | | | appropriate | |
| Hypothyroidism | Grade 2, 3 or 4 | Continue | Initiate thyroid | Monitor for signs and |
| | | | replacement | symptoms of |
| | | | hormones (e.g., | thyroid disorders |
| | | | levothyroxine or | |
| | | | liothyronine) per | |
| | | | standard of care | |
| Nephritis: | Grade 2 | Withhold | Administer | Monitor changes of |
| grading | Grade 3 or 4 | Permanently | corticosteroids | renal function |
| according to | | discontinue | (prednisone | |
| increased | | | 1 to 2 mg/kg or | |
| creatinine or | | | equivalent) | |
| acute kidney | | | followed by taper | |
| injury | | | | |
| Neurological | Grade 2 | Withhold | Based on severity of AE | Ensure adequate |
| Toxicities | Grade 3 or 4 | Permanently | administer | evaluation to |
| | | discontinue | corticosteroids | confirm etiology |
| | | | | and/or exclude |
| | | | | other causes |
| Myocarditis | Grade 2, 3 or 4 | Permanently | Based on severity of AE | Ensure adequate |
| | | discontinue | administer | evaluation to |
| | | | corticosteroids | confirm etiology |
| | | | | and/or exclude |
| | | | | other causes |
| Exfoliative | Suspected SJS, | Withhold | Based on severity of AE | Ensure adequate |
| Dermatologic | TEN, or | | administer | evaluation to |
| Conditions | DRESS | | corticosteroids | confirm etiology or |
| | Confirmed SJS, | Permanently | | exclude other |
| | TEN, or | discontinue | | causes |
| | DRESS | | | |
| All Other irAEs | Persistent | Withhold | Based on severity of AE | Ensure adequate |
| | Grade 2 | | administer | evaluation to |
| | Grade 3 | Withhold or | corticosteroids | confirm etiology or |
| | | discontinue | | exclude other |
| | | based on the | | causes |
| | | event ^{e} | | |
| | Recurrent | Permanently | | |
| | Grade 3 or | discontinue | | |
| | Grade 4 | | | |

AE(s) = adverse event(s);
ALT = alanine aminotransferase;
AST = aspartate aminotransferase;
CTCAE = Common Terminology Criteria for Adverse Events;
DRESS = Drug Rash with Eosinophilia and Systemic Symptom;
GI = gastrointestinal;
IO = immuno-oncology;
ir = immune related;
IV = intravenous;
SJS = Stevens-Johnson Syndrome;
T1DM = type 1 diabetes mellitus;
TEN = Toxic Epidermal Necrolysis;
ULN = upper limit of normal.
Note:
Non-irAE will be managed as appropriate, following clinical practice recommendations.
^{a} AST/ALT: >3.0 to 5.0 × ULN if baseline normal; >3.0 to 5.0 × baseline, if baseline abnormal; bilirubin: >1.5 to 3.0 × ULN if baseline normal; >1.5 to 3.0 × baseline if baseline abnormal
^{b} AST/ALT: >5.0 to 20.0 × ULN, if baseline normal; >5.0 to 20.0 × baseline, if baseline abnormal; bilirubin: >3.0 to 10.0 × ULN if baseline normal; >3.0 to 10.0 × baseline if baseline abnormal
^{c} AST/ALT: >20.0 × ULN, if baseline normal; >20.0 × baseline, if baseline abnormal; bilirubin: >10.0 × ULN if baseline normal; >10.0 × baseline if baseline abnormal
^{d} The decision to withhold or permanently discontinue pembrolizumab is at the discretion of the investigator or treating physician. If control achieved or ≤Grade 2, pembrolizumab may be resumed.
^{e} Events that require discontinuation include but are not limited to: encephalitis and other clinically important irAEs (e.g., vasculitis and sclerosing cholangitis).

Dose modification and toxicity management guidelines on pembrolizumab associated infusion reaction are provided in Table 3.

TABLE 3
Pembrolizumab infusion reaction dose modification and treatment
guidelines

| | | Premedication at Subsequent |
|---|---|---|
| NCI CTCAE Grade | Treatment | Dosing |
| Grade 1 | Increase monitoring of vital signs as | None |
| Mild reaction; infusion | medically indicated until the | |
| interruption not indicated; | participant is deemed medically | |
| intervention not indicated | stable in the opinion of the | |
| | investigator. | |
| Grade 2 | Stop Infusion. | Participant may be premedicated |
| Requires therapy or infusion | Additional appropriate medical | 1.5 h (±30 minutes) prior to |
| interruption but responds | therapy may include but is not | infusion with: |
| promptly to symptomatic | limited to: | Diphenhydramine 50 mg po |
| treatment (e.g., antihistamines, | IV fluids | (or equivalent dose of |
| NSAIDs, narcotics, IV fluids); | Antihistamines | antihistamine). |
| prophylactic medications | NSAIDs | Acetaminophen 500-1000 mg |
| indicated for ≤24 hrs. | Acetaminophen | po (or equivalent dose of |
| | Narcotics | analgesic). |
| | Increase monitoring of vital | |
| | signs as medically indicated | |
| | until the participant is deemed | |
| | medically stable in the opinion | |
| | of the investigator. | |
| | If symptoms resolve within 1 | |
| | hour of stopping drug infusion, | |
| | the infusion may be restarted at | |
| | 50% of the original infusion | |
| | rate (e.g., from 100 mL/hr. to 50 | |
| | mL/hr.). Otherwise dosing will | |
| | be held until symptoms resolve | |
| | and the participant should be | |
| | premedicated for the next | |
| | scheduled dose. | |
| | Participants who develop Grade 2 | |
| | toxicity despite adequate | |
| | premedication should be | |
| | permanently discontinued from | |
| | further study drug treatment | |
| Grades 3 or 4 | Stop Infusion. | No subsequent dosing |
| Grade 3: | Additional appropriate medical | |
| Prolonged (i.e., not rapidly | therapy may include but is not | |
| responsive to symptomatic | limited to: | |
| medication and/or brief | Epinephrine** | |
| interruption of infusion); | IV fluids | |
| recurrence of symptoms | Antihistamines | |
| following initial improvement; | NSAIDs | |
| hospitalization indicated for | Acetaminophen | |
| other clinical sequelae (e.g., | Narcotics | |
| renal impairment, pulmonary | Oxygen | |
| infiltrates) | Pressors | |
| Grade 4: | Corticosteroids | |
| Life-threatening; pressor or | Increase monitoring of vital | |
| ventilator support indicated | signs as medically indicated | |
| | until the participant is deemed | |
| | medically stable in the opinion | |
| | of the investigator. | |
| | Hospitalization may be | |
| | indicated. | |
| | **In cases of anaphylaxis, epinephrine | |
| | should be used immediately. | |
| | Participant is permanently | |
| | discontinued from further study | |
| | drug treatment. | |

Appropriate resuscitation equipment should be available at the bedside and a physician readily available during the period of drug administration.

The cohorts that eligible subjects who had radiographic-confirmed progression on PD-1 inhibitor therapy are being split into are as follows:
Cohort 1: subjects with primary refractory metastatic NSCLC (without known targetable EGFR, ALK, ROS1, or BRAF mutations) and progressed while on adjuvant anti-PD-1 therapy or have SD <6 months on each prior line of anti-PD-1 containing therapy
Cohort 2: subjects with relapsed metastatic NSCLC (without known targetable EGFR, ALK, ROS1, or BRAF mutations) and progressed after having developed a prior objective response of CR/PR for at least 3 months or SD for at least 6 months to any prior line of PD-1 containing therapy. Adjuvant courses of therapy without measurable disease do not apply to the SD for at least 6 months criteria
Cohort 3: subjects with primary refractory metastatic cutaneous melanoma (including subjects without targetable gene mutations and BRAF-V600E/V600K subjects who have failed targeted therapy) and progressed while on adjuvant anti-PD-1 therapy or have SD <6 months on each prior line of anti-PD-1 containing therapy
Cohort 4: subjects with relapsed metastatic cutaneous melanoma (including subjects without targetable gene mutations and BRAF-V600E/V600K subjects who have failed targeted therapy) after having developed a prior objective response of CR/PR for at least 3 months or SD for at least 6 months to any prior line of PD-1 containing therapy. Adjuvant courses of therapy without measurable disease do not apply to the SD for at least 6 months criteria

Based on antitumor activity and safety data, each cohort may have an expansion phase (up to an additional 40 subjects per cohort) to further characterize the safety and antitumor activity of brentuximab vedotin with pembrolizumab.

Response is being assessed by computed tomography (CT) scans or magnetic resonance imaging (MRI) every 6 weeks from first dose until Week 48 for all subjects, then every 12 weeks thereafter until disease progression. CT scans with and without contrast will be performed on the chest, abdomen, and pelvis. Subjects with a history of brain metastasis should have brain MRI with contrast approximately every 12 weeks from the date of first dose or sooner if clinically indicated. Subjects will be followed for safety for 30 days after the last dose of study drug. 11/15 subjects with relapsed melanoma and 6/15 subjects with relapsed NSCLC have been evaluated for efficacy as described in this example. 4/15 subjects with primary refractory melanoma and 1/15 subject with primary refractory NSCLC have been evaluated for efficacy as described in this example.

### Inclusion Criteria

1. Subjects with relapsed or refractory metastatic squamous or nonsquamous NSCLC (without known targetable EGFR, ALK, ROS1, or BRAF mutations) or metastatic cutaneous melanoma (including subjects without targetable gene mutations and BRAF-V600E/V600K subjects who have failed targeted therapy).
2. Subjects must be currently on PD-1 checkpoint inhibitor (CPI) therapy (e.g. nivolumab or pembrolizumab) or had their last dose of PD-1 CPI therapy within 90 days prior to enrollment; PD-1 inhibitor therapy must be the last previous line of therapy.
3. Have measurable disease per RECIST 1.1 as assessed by the local site investigator/radiology. Lesions situated in a previously irradiated area are considered measurable if progression has been demonstrated in such lesions

### Either 4A or 4B

4A. Subjects must have progressed on treatment with an anti-PD-1 monoclonal antibody (mAb) administered either as monotherapy, or in combination with other checkpoint inhibitors or other therapies. PD-1 treatment progression is defined by meeting all of the following criteria.
   a. Subjects with refractory disease must have progressed while on adjuvant anti-PD-1 therapy or for those with measurable disease without a prior objective response or with SD for <6 months on each prior line of anti-PD1 containing therapy.
   b. Subjects with relapsed diseased must have progressed after having developed a prior objective response of CR/PR for at least 3 months or SD for at least 6 months to any prior line of PD-1 containing therapy. Adjuvant courses of therapy without measurable disease do not apply to the SD for at least 6 months criteria.
4B. Subjects must have progressed on treatment in the metastatic setting with an anti-PD-1 monoclonal antibody (mAb) administered either as monotherapy, or in combination with other checkpoint inhibitors or other therapies. PD-1 treatment progression is defined by meeting all of the following criteria.
   a. Subjects with refractory disease must have progressed without a prior objective response during or after prior PD-1 inhibitor therapy within 3 months or have SD for <6 months.
   OR
   b. Subjects with relapsed diseased must have progressed after having developed a prior objective response of CR/PR for at least 3 months or SD for at least 6 months.
   AND
   c. Has received at least 2 doses of an approved anti-PD-1 mAb.
      i. Has demonstrated disease progression (PD) after PD-1 as defined by RECIST v1.1. Progressive disease has been documented within 90 days from the last dose of anti-PD-1 mAb.
      ii. Progressive disease prior to enrollment is determined according to iRECIST for patients on anti-PD-1 containing therapy for <12 weeks. For patients with <12 weeks of anti-PD-1 containing therapy initial evidence of PD is to be confirmed by a second assessment no less than 4 weeks from the date of the first documented PD, in the absence of rapid clinical progression.
      iii. This determination is made by the investigator. Once PD is confirmed, the initial date of PD documentation will be considered the date of disease.
5. Tumor tissue sample obtained within 3 months is required prior to enrollment. Bone biopsies and aspirates are not an acceptable source of tumor tissue. If an archival tumor sample is not available, a fresh biopsy sample must be collected prior to the first dose of study drug, unless medically infeasible and with prior agreement of the medical monitor.
6. Age 18 years or older.
7. An Eastern Cooperative Oncology Group (ECOG) Performance Status score of equal or less than 1.
8. The following baseline laboratory data within 10 days prior to the start of study treatment:
   absolute neutrophil count (ANC) ≥1500/μL
   platelet count ≥100,000/μL
   Hemoglobin ≥9.0 g/dL or ≥5.6 mmol/L
   serum bilirubin ≤1.5× upper limit of normal (ULN) or direct bilirubin ≤ULN for participants with total bilirubin levels >1.5×ULN
   estimated glomerular filtration rate (GFR) ≥45 mL/min/1.73 m² using the Modification of Diet in Renal Disease (MDRD) study equation as applicable.
   alanine aminotransferase (ALT) and aspartate aminotransferase (AST)≤2.5×ULN (≤5×ULN for participants with liver metastases)
   International normalized ratio (INR) OR prothrombin time (PT)≤1.5×ULN unless participant is receiving anticoagulant therapy as long as PT or activated partial thromboplastin time (aPTT) is within therapeutic range of intended use of anticoagulants
9. The following requirements for subjects who are human immunodeficiency virus (HIV) positive:
   CD4+ T cell counts ≥350 cell/μL.
   No AIDS-defining opportunistic infection within the past 12 months
   On established highly active antiretroviral therapy (HAART) and have achieved and maintained virologic suppression defined as confirmed HIV RNA level below 50 copies/mL
10. Subjects of childbearing potential under the following conditions:
   a. Must have a negative serum or urine pregnancy test (minimum sensitivity 25 mIU/mL or equivalent units of beta human chorionic gonadotropin [β-hCG]) result within 7 days prior to the first dose of study drug. Subjects with false positive results and documented verification that the subject is not pregnant are eligible for participation
   b. Must agree not to try to become pregnant during the study and for at least 120 days after the final dose of pembrolizumab or brentuximab vedotin, whichever is later
   c. Must agree not to breastfeed or donate ova, starting at time of informed consent and continuing through 120 days after the final dose of pembrolizumab or brentuximab vedotin, whichever is later.
   d. If sexually active in a way that could lead to pregnancy, must consistently use 2 highly effective methods of birth control starting at time of informed consent and continuing throughout the study and for at least 120 days after the final dose of pembrolizumab or brentuximab vedotin, whichever is later
11. Subjects who can father children, under the following conditions:
   a. Must agree not to donate sperm starting at time of informed consent and continuing throughout the study period and for at least 120 days after the final dose of pembrolizumab or brentuximab vedotin, whichever is later.
   b. If sexually active with a person of childbearing potential in a way that could lead to pregnancy, must consistently use 2 highly effective methods of birth control starting at time of informed consent and continuing throughout the study and for at least 120 days after the final dose of pembrolizumab or brentuximab vedotin, whichever is later.
   c. If sexually active with a person who is pregnant or breastfeeding, must consistently use one of 2 contraception options starting at time of informed consent and continuing throughout the study and for at least 120 days after the final dose of pembrolizumab or brentuximab vedotin, whichever is later.

### Exclusion Criteria

1. Has known active CNS metastases and/or carcinomatous meningitis. Participants with previously treated brain metastases may participate provided they are radiologically stable, i.e., without evidence of progression for at least 4 weeks by repeat imaging (note that the repeat imaging should be performed during study screening), clinically stable and without requirement of steroid treatment for at least 14 days prior to first dose of study treatment.
2. Prior immunosuppressive chemotherapy, therapeutic radiation, or any immunotherapy (e.g., immunoglobulin replacement, other monoclonal antibody therapies) within 4 weeks of first study drug dose.
3. History of another malignancy within 3 years before the first dose of study drug or any evidence of residual disease from a previously diagnosed malignancy. Exceptions are malignancies with a negligible risk of metastasis or death (e.g., 5-year overall survival ≥90%), such as adequately treated carcinoma in situ of the cervix, non-melanoma skin carcinoma, localized prostate cancer, ductal carcinoma in situ, or Stage I uterine cancer.
4. History of progressive multifocal leukoencephalopathy (PML).
5. Active cerebral/meningeal disease related to the underlying malignancy. Subjects with a history of cerebral/meningeal disease related to the underlying malignancy are allowed if prior CNS disease has been treated definitively.
6. Any uncontrolled Grade 3 or higher (per the National Cancer Institute's Common Terminology Criteria for Adverse Events, NCI CTCAE Version 5.0) viral, bacterial, or fungal infection within 2 weeks prior to the first dose of study drug. Routine antimicrobial prophylaxis is permitted.
7. Subjects who are breastfeeding.
8. Known hypersensitivity to any excipient contained in the drug formulation of brentuximab vedotin or pembrolizumab.
9. Known to be positive for hepatitis B by surface antigen expression. Known to be positive for hepatitis C infection (positive by polymerase chain reaction or on antiviral therapy for hepatitis C within the last 6 months). Subjects who have been treated for hepatitis C infection are permitted if they have documented sustained virologic response of 12 weeks.
10. Previous treatment with brentuximab vedotin.
11. Current therapy with other systemic anti-neoplastic or investigational agents.
12. Grade 3 or higher pulmonary disease unrelated to underlying malignancy.
13. Documented history of a cerebral vascular event (stroke or transient ischemic attack), unstable angina, myocardial infarction, or cardiac symptoms consistent with New York Heart Association Class III-IV within 6 months prior to their first dose of brentuximab vedotin.
14. Congestive heart failure, Class III or IV, by the New York Heart Association criteria.
15. Grade 2 or higher peripheral sensory or motor neuropathy at baseline.
16. Idiopathic interstitial pneumonia or diffusing capacity of the lung for carbon monoxide (DLCO; adjusted for hemoglobin)<50% predicted.
12. Has a diagnosis of immunodeficiency or is receiving chronic systemic steroid therapy (in dosing exceeding 10 mg daily of prednisone equivalent) or any other form of immunosuppressive therapy within 7 days prior the first dose of study drug.
13. Has an active autoimmune disease that has required systemic treatment in past 2 years (i.e., with use of disease modifying agents, corticosteroids or immunosuppressive drugs). Replacement therapy (e.g., thyroxine, insulin, or physiologic corticosteroid replacement therapy for adrenal or pituitary insufficiency) is not considered a form of systemic treatment and is allowed.
14. HIV-infected participants with a history of Kaposi sarcoma and/or Multicentric Castleman Disease.
15. Has received prior radiotherapy within 2 weeks of start of study treatment. Participants must have recovered from all radiation-related toxicities, not require corticosteroids, and not have had radiation pneumonitis. A 1-week washout is permitted for palliative radiation (≤2 weeks of radiotherapy) to non-CNS disease.
16. Has received a live vaccine within 30 days prior to the first dose of study drug. Examples of live vaccines include, but are not limited to, the following: measles, mumps, rubella, varicella/zoster (chicken pox), yellow fever, rabies, Bacillus Calmette-Guérin (BCG), and typhoid vaccine. Seasonal influenza vaccines for injection are generally killed virus vaccines and are allowed; however, intranasal influenza vaccines (e.g., FluMist®) are live attenuated vaccines and are not allowed.
17. Has a history of (non-infectious) pneumonitis that required steroids or has current pneumonitis.
18. For NSCLC subjects: Has received radiation therapy to the lung that is >30 Gy within 6 months of the first dose of trial treatment
19. Has a history or current evidence of any condition, therapy, or laboratory abnormality that might confound the results of the study, interfere with the participant's participation for the full duration of the study, or is not in the best interest of the participant to participate, in the opinion of the treating investigator.
20. Has a known psychiatric or substance abuse disorder that would interfere with the participant's ability to cooperate with the requirements of the study.
21. Has had an allogenic tissue/solid organ transplant.
22. Has received prior therapy with an anti-PD-1, anti-PD-L1, or anti PD-L2 agent or with an agent directed to another stimulatory or co-inhibitory T-cell receptor (e.g., CTLA-4, OX 40, CD137) and was discontinued from that treatment due to a Grade 3 or higher immune-related adverse event (irAE).
23. Other serious underlying medical condition that, in the opinion of the investigator, would impair the subject's ability to receive or tolerate the planned treatment and follow-up.

### Response/Efficacy Assessment

Response is being assessed by radiographic tumor evaluation every 6 weeks until Week 48, then every 12 weeks thereafter until disease progression. Response assessments should be calculated from Cycle 1 Day 1.

Tumor evaluation is being performed by CT scan of the chest, abdomen, and pelvis. A CT of the neck must also be obtained if there is documented or suspected involvement in this region. CT scans must be of diagnostic quality, and IV contrast must be used unless medically contraindicated. Should a subject be unable to be evaluated by CT, MRI is allowed if used consistently for all protocol-required response/efficacy assessments. Subjects with a history of brain metastasis should have brain MRI with contrast approximately every 12 weeks from the date of first dose or sooner if clinically indicated.

Peripheral blood and tumor biopsies are being collected at protocol specified time points. A biopsy is required at baseline and 6 weeks after the first dose (+1 week; at least 1 week after dosing preferred). If a biopsy at 6 weeks is not feasible, the biopsy may be waived after discussion with the medical monitor. Archival tumor tissue is acceptable at baseline if it was collected within 3 months prior to enrollment and after any systemic therapy. If archival tumor tissue is not available, a baseline tumor biopsy is required unless medically infeasible and with prior agreement of the medical monitor. An optional tumor biopsy will be collected at the time of disease progression or end of treatment. Only soft tissue biopsies (e.g., excisional, core needle, punch) are acceptable. Fine needle aspirations and bone biopsies are not acceptable.

Exploratory, predictive, and prognostic biomarkers associated with response, resistance, or safety observations are being evaluated before and during treatment with brentuximab vedotin and CPI therapy. On treatment biopsies are being used to characterize the MOA and resistance mechanisms of brentuximab vedotin plus pembrolizumab therapy.

Biomarker assessments in peripheral blood may include, but are not limited to, measurement of baseline and drug-induced changes in circulating blood cell subpopulations (CD8, CD4, Tregs, and MDSC), immune mediators (cytokines, chemokines, and other soluble factors such as sCD30, sCD153, sPD-1 and sPD-L1), and circulating disease markers. Brentuximab vedotin interactions with peripheral blood cells and tissues may also be monitored. Assays may include, but are not limited to, flow cytometry, ELISA, Luminex, next generation sequencing, proteomic methodologies, and other immunoassays. The flow cytometry staining panels for evaluation of Treg, MDSC, and activated and proliferating CD4 and CD8 T cells are listed below:
Treg panel: CD3/CD4/CD8/CD25/CD127/CD30/CD153/CD183/CD194/CD196
MDSC panel: CD33/CD3-CD19-CD56/CD15/CD16/CD14/HLA-DR/CD11b/CD66b/CD45
CD4 and CD8 panel: CD3/CD4/CD8/Ki67/HLA-DR/CD30/CD153/PD-1/PD-L1

Biomarker assessments in tumor tissue may include, but are not limited to, measurements of CD30, PD-L1, Foxp3 (Treg), CD8, MDSC and other immune cell or tumor cell markers, brentuximab vedotin and its potential metabolites as well as characterization of the tumor microenvironment, tumor subtyping, profiling of somatic mutations or alterations in genes or RNA commonly altered in cancer, and drug effects. Assays may include, but are not limited to, immunohistochemistry staining and next generation sequencing of RNA and DNA.

Subjects dosed with at least one dose of either study drug are being included in the safety assessments. Safety assessments consist of the surveillance and recording of adverse events and measurements of physical examination findings and laboratory tests.

The study objectives and corresponding endpoints are summarized in Table 4.

TABLE 4
Objectives and corresponding endpoints

| Primary Objective | Corresponding Primary Endpoint |
|---|---|
| Evaluate the objective response rate (ORR) of | Confirmed ORR based on investigator assessment using |
| the combination of brentuximab vedotin with | RECIST 1.1 criteria |
| pembrolizumab | |
| Secondary Objectives | Corresponding Secondary Endpoints |
| Evaluate the duration of response (DOR) of the | DOR based on investigator assessment using RECIST |
| combination of brentuximab vedotin with | 1.1 criteria |
| pembrolizumab | PFS based on investigator assessment using RECIST 1.1 |
| Evaluate the progression-free survival (PFS) of | criteria from the date of the first dose to the first disease |
| the combination of brentuximab vedotin with | progression or death from any cause. |
| pembrolizumab | ORR and DOR per iRECIST by investigator assessment |
| Evaluate ORR and DOR per the modified | Incidence, severity, seriousness, and relatedness of |
| RECIST v1.1 for immune-based therapeutics | treatment-emergent adverse events (AEs) per National |
| (iRECIST) | Cancer Institute Common Terminology Criteria for |
| Evaluate the safety of the combination of | Adverse Events (NCI CTCAE) version 5.0 |
| brentuximab vedotin with pembrolizumab | |
| Exploratory Objectives | Corresponding Exploratory Endpoints |
| Evaluate the overall survival (OS) | OS |
| Evaluate changes in Treg and MDSC levels in | Treg and MDSC levels in peripheral blood and tumor |
| peripheral blood and tumor tissue | tissue before and after treatment |
| Evaluate changes in CD8+ T cell level in | CD8+ T cell proliferation in peripheral blood and |
| peripheral blood and tumor tissue | infiltration in tumor tissue before and after treatment |
| Evaluate the potential association of CD30 and | Efficacy outcomes in tumors with different expression |
| PD-L1 expression and clinical response | levels of CD30 and PD-L1 |
| Evaluate the health-related quality of life of | Patient-reported outcomes assessed by the EQ-5D-5L |
| brentuximab vedotin in combination with | questionnaire |
| pembrolizumab based on health status collected | |
| using the EQ-5D-5L questionnaire | |

The primary endpoint of this study is the confirmed ORR per RECIST v1.1 based on investigator assessment. Confirmed ORR per RECIST v1.1 is defined as the proportion of subjects whose best overall response is a confirmed CR or PR according to RECIST v1.1. Subjects who do not have at least 2 (initial response and confirmation scan) post-baseline response assessments will be scored as non-responders for calculating the confirmed ORR. A separate secondary endpoint of ORR per iRECIST summarizing the proportion of subjects with confirmed CR or PR based on iRECIST guidelines will also be analyzed.

DOR per RECIST v1.1 is defined as the time from start of the first documentation of confirmed objective tumor response (CR or PR) per RECIST v1.1 to the first documentation of PD (per RECIST v1.1) or to death due to any cause, whichever comes first. Subjects without progression per RECIST v1.1 or death will be censored; details will be provided in the statistical analysis plan (SAP). DOR per RECIST v1.1 will only be calculated for the subset of subjects achieving a CR or PR.

A separate endpoint of DOR per iRECIST is also being analyzed, where DOR per iRECIST is defined as the time from first documentation of confirmed objective response (CR or PR) based on iRECIST guidelines by investigator assessment to the first documentation of confirmed objective tumor progression per iRECIST by investigator assessment, or to death due to any cause, whichever comes first.

PFS is defined as the time from start of study treatment to first documentation of objective tumor progression (PD per RECIST v1.1), or to death due to any cause, whichever comes first. PFS data will be censored on the date of the last radiological assessment of measured lesions documenting absence of PD for subjects who do not have objective tumor progression and are still on study at the time of an analysis, are given antitumor treatment other than the study treatment, or are removed from study prior to documentation of objective tumor progression. Subjects lacking an evaluation of tumor response after their first dose will have their event time censored at 1 day.

Overall survival (OS) is defined as the time from first dose to death due to any cause. Specifically,

$$OS = Date of death - Date of first dose + 1$$

For a subject who is not known to have died by the end of study follow-up, observation of OS is censored on the date the subject was last known to be alive (i.e., date of last contact). Subjects lacking data beyond the day of first dose will have their survival time censored on the date of first dose (i.e., OS duration of 1 day).

Biomarkers in both the peripheral blood and tumor tissue are being analyzed. Peripheral blood is being collected at baseline and different time points post-treatment. The levels of T regulatory cells (Treg), myeloid derived suppressor cells (MDSC), CD8 T cells and other immune subsets are being measured by flow cytometry using established staining panels. Potential changes in the percentage or absolute number of each immune subset over time are being evaluated. In tumor tissue, the levels of CD30, PD-L1, Treg, and CD8 T cells are being measured using immunohistochemistry (IHC) staining and gene expression analysis. The levels of CD30, Treg, PD-L1, and CD8 are being compared between baseline and post-treatment biopsy samples for potential changes in the tumor microenvironment. The potential association between baseline tumor expression of CD30 and ORR is being investigated.

### Results

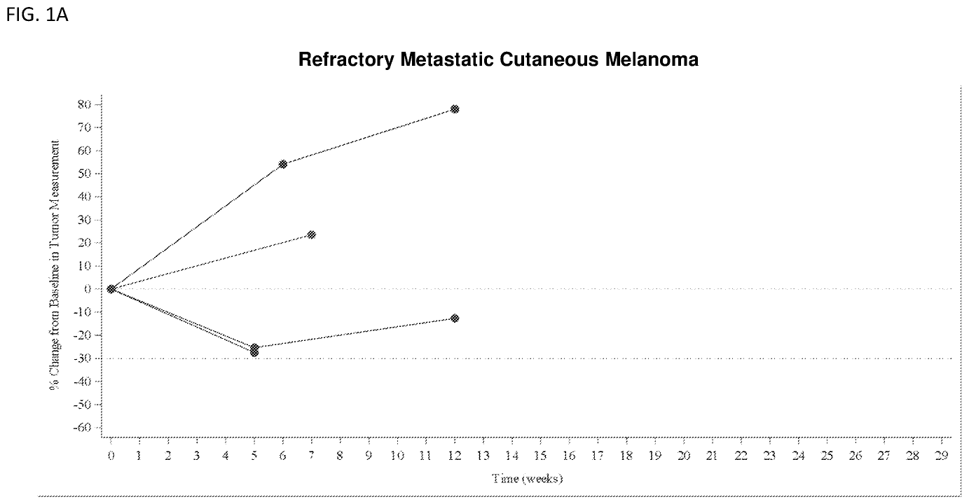

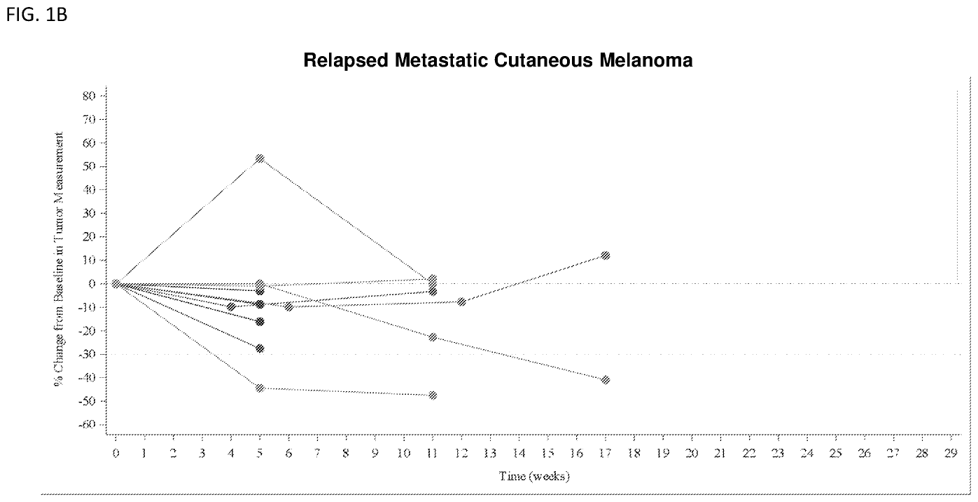

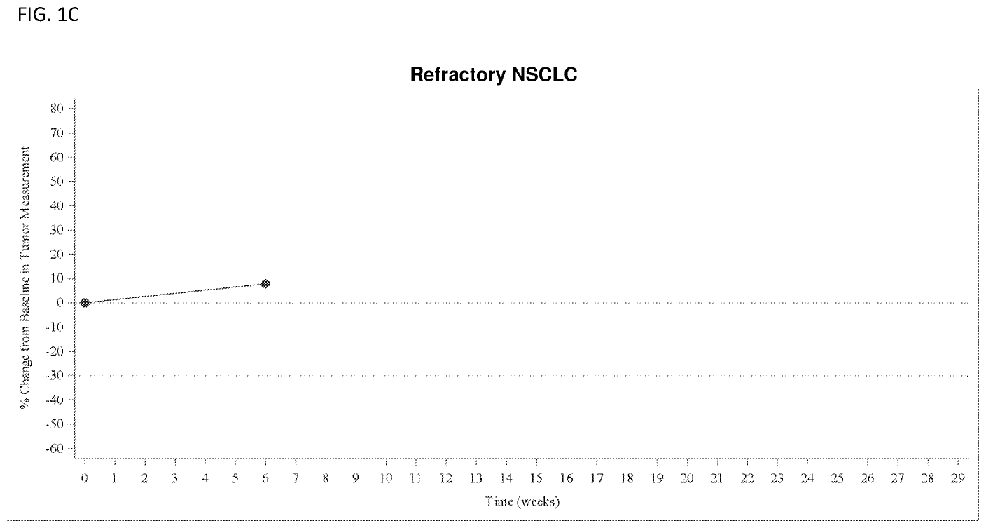

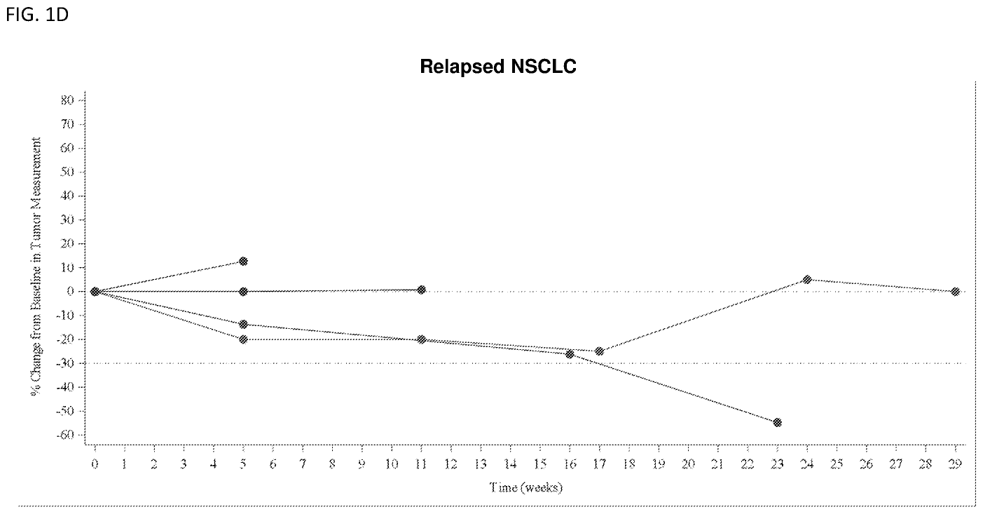

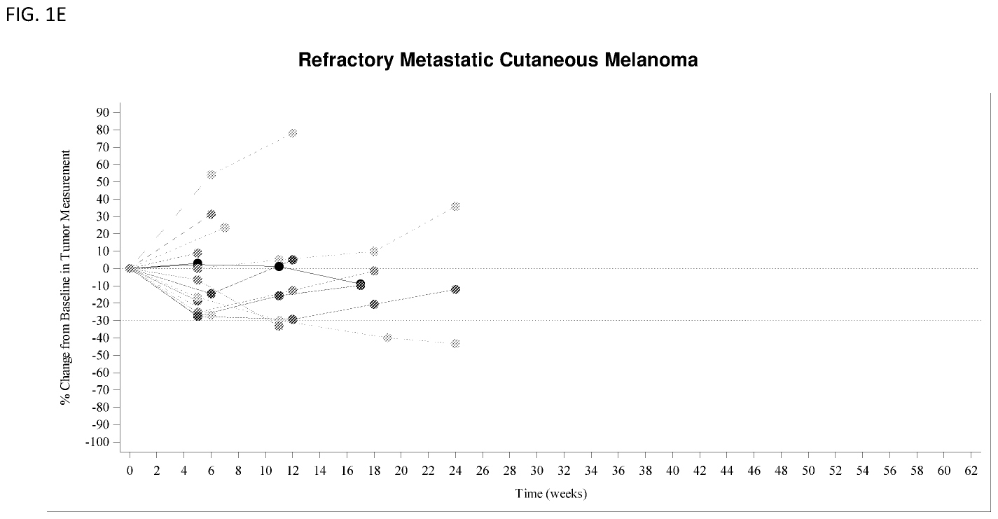

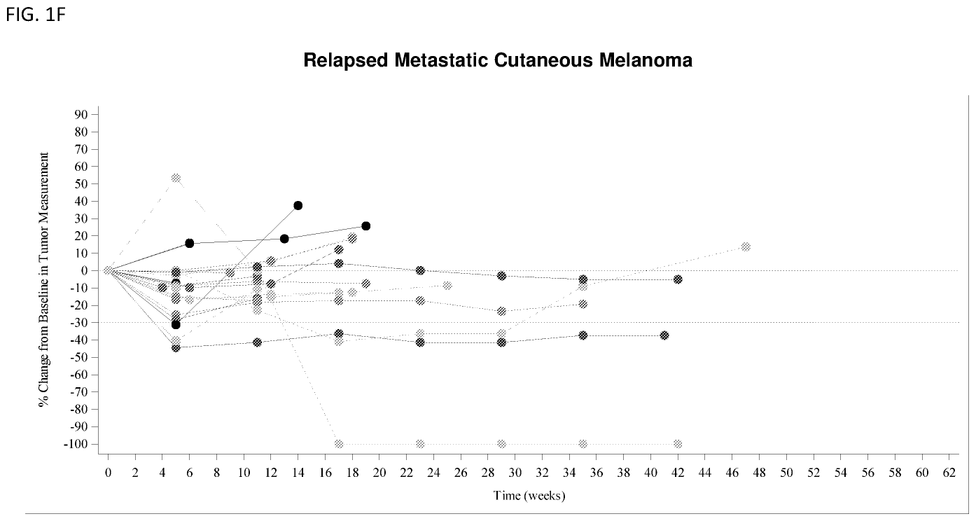

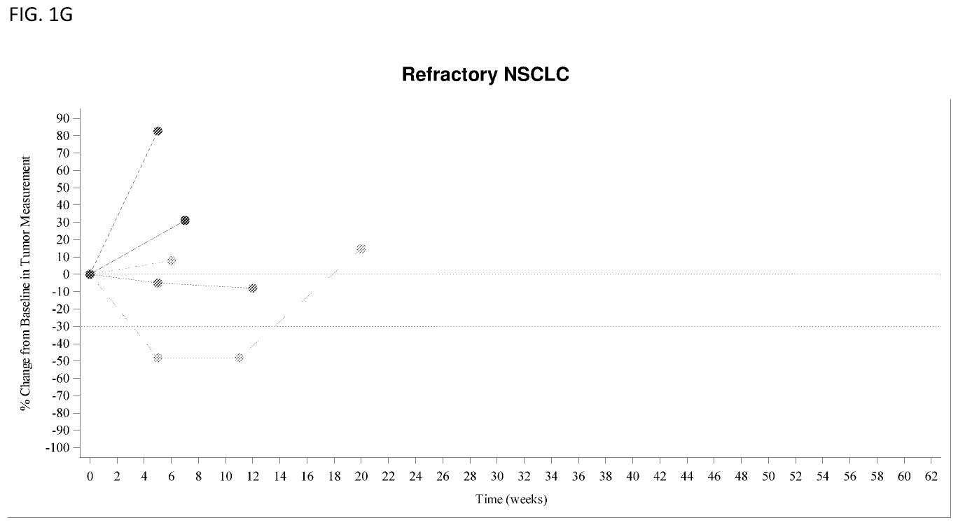

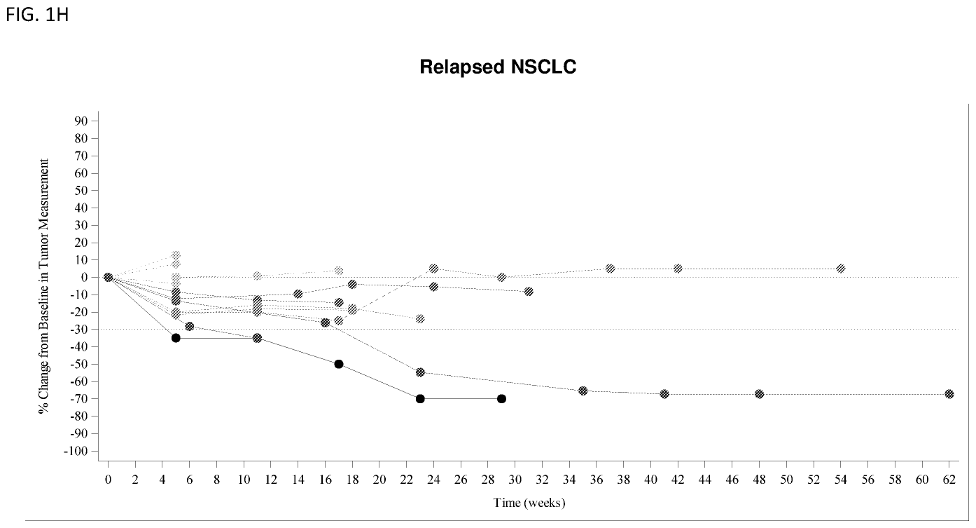

FIG. 1A and FIG. 1E show the percent change from baseline in tumor measurement for the subjects with refractory metastatic cutaneous melanoma that have been evaluated for efficacy. For the best overall response in FIG. 1A, two subjects exhibited SD and two subjects exhibited PD. FIG. 1B and FIG. 1F show the percent change from baseline in tumor measurement for the subjects with relapsed metastatic cutaneous melanoma that have been evaluated for efficacy. For the best overall response in FIG. 1B, three subjects exhibited PR, seven subjects exhibited SD and one subject exhibited PD. FIG. 1C and FIG. 1G show the percent change from baseline in tumor measurement for the subjects with refractory NSCLC that has been evaluated for efficacy. For the best overall response in FIG. 1C, one subject exhibited PD. FIG. 1D and FIG. 1H show the percent change from baseline in tumor measurement for the subjects with relapsed NSCLC that have been evaluated for efficacy. For the best overall response in FIG. 1D, one subject exhibited PR, four subjects exhibited SD, and one subject exhibited PD. Each line in FIG. 1A-1H represents an individual subject.

### Example 2: A Phase II Trial of Brentuximab Vedotin in Combination with a Pembrolizumab in Subjects with Metastatic Head and Neck Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic head and neck squamous cell carcinoma. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 3: A Phase II Trial of Brentuximab Vedotin in Combination with a Pembrolizumab in Subjects with Metastatic Renal Cell Carcinoma

An additional open-label, multi-cohort, multicenter, phase 2 clinical trial designed to evaluate the safety and antitumor activity of brentuximab vedotin in combination with pembrolizumab for the treatment of subjects with relapsed metastatic renal cell carcinoma. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 4: A Phase II Trial of Brentuximab Vedotin in Combination with a Pembrolizumab in Subjects with Metastatic Bladder Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic bladder cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 5: A Phase II Trial of Brentuximab Vedotin in Combination with a Pembrolizumab in Subjects with a Microsatellite Instability-High or Mismatch Repair Deficient Cancer

An additional open-label, multi-cohort, multicenter, phase 2 clinical trial designed to evaluate the safety and antitumor activity of brentuximab vedotin in combination with pembrolizumab for the treatment of subjects with a microsatellite instability-high or mismatch repair deficient cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 6: A Phase II Trial of Brentuximab Vedotin in Combination with a Pembrolizumab in Subjects with Metastatic Colorectal Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic colorectal cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 7: A Phase II Trial of Brentuximab Vedotin in Combination with a Pembrolizumab in Subjects with Metastatic Gastric Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic gastric cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 8: A Phase II Trial of Brentuximab Vedotin in Combination with a Pembrolizumab in Subjects with Metastatic Esophageal Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic esophageal cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 9: A Phase II Trial of Brentuximab Vedotin in Combination with a Pembrolizumab in Subjects with Metastatic Cervical Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic cervical cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 10: A Phase II Trial of Brentuximab Vedotin in Combination with a Pembrolizumab in Subjects with Metastatic Hepatocellular Carcinoma

An additional open-label, multi-cohort, multicenter, phase 2 clinical trial designed to evaluate the safety and antitumor activity of brentuximab vedotin in combination with pembrolizumab for the treatment of subjects with relapsed metastatic hepatocellular carcinoma. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 11: A Phase II Trial of Brentuximab Vedotin in Combination with a Pembrolizumab in Subjects with Metastatic Endometrial Carcinoma

An additional open-label, multi-cohort, multicenter, phase 2 clinical trial designed to evaluate the safety and antitumor activity of brentuximab vedotin in combination with pembrolizumab for the treatment of subjects with relapsed metastatic endometrial carcinoma. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 12: A Phase II Trial of Brentuximab Vedotin in Combination with a Pembrolizumab in Subjects with Metastatic Merkel Cell Carcinoma

An additional open-label, multi-cohort, multicenter, phase 2 clinical trial designed to evaluate the safety and antitumor activity of brentuximab vedotin in combination with pembrolizumab for the treatment of subjects with relapsed metastatic Merkel cell carcioma. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 13: A Phase II Trial of Brentuximab Vedotin in Combination with a Pembrolizumab in Subjects with Metastatic Cutaneous Squamous Cell Carcinoma

An additional open-label, multi-cohort, multicenter, phase 2 clinical trial designed to evaluate the safety and antitumor activity of brentuximab vedotin in combination with pembrolizumab for the treatment of subjects with relapsed metastatic cutaneous squamous cell carcinoma. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 14: A Phase II Trial of Brentuximab Vedotin in Combination with a Pembrolizumab in Subjects with Metastatic Breast Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic breast cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 15: A Phase II Trial of Brentuximab Vedotin in Combination with a Pembrolizumab in Subjects with Metastatic Tumor Mutational Burden-High Cancer

An additional open-label, multi-cohort, multicenter, phase 2 clinical trial designed to evaluate the safety and antitumor activity of brentuximab vedotin in combination with pembrolizumab for the treatment of subjects with relapsed metastatic tumor mutational burden-high cancer. Subjects will be treated with brentuximab vedotin 1.8 mg/kg via intravenous infusion every 3 weeks in combination with pembrolizumab 200 mg every 3 weeks as described in Example 1. Response and efficacy will be evaluated as described in Example 1. The objectives and corresponding endpoints will be as described in Example 1.

### Example 16: CD30 Expression is Restricted to Activated Tregs in Syngeneic A20 Tumors

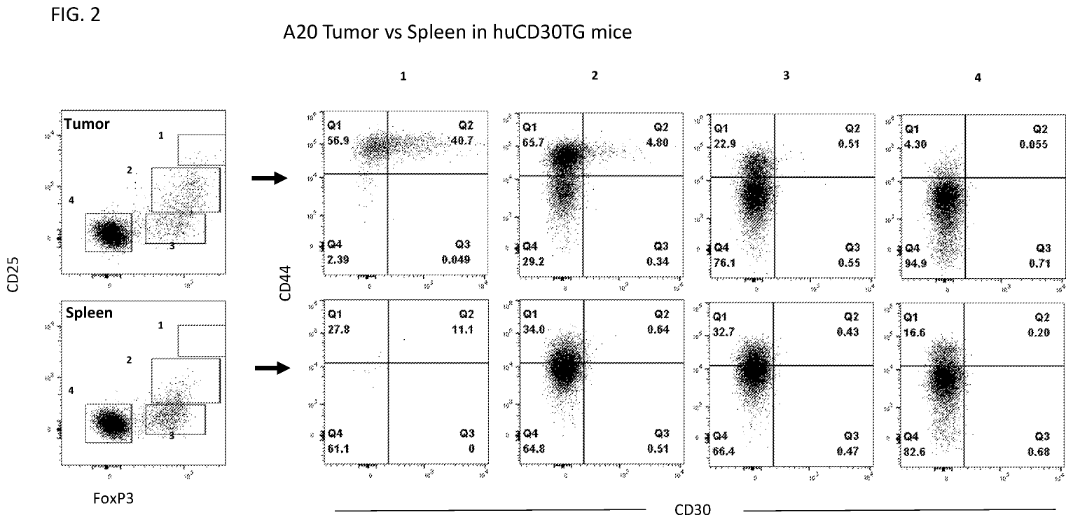

A20 tumors were implanted subcutaneous (s.c.) into the left flank of mice engineered to express one copy of human CD30 (TNFRSF8) in place of murine CD30, huCD30 Tg+/− mice. Tumors and spleens were harvested from mice when tumors reached 200 mm³ and analyzed by flow cytometry for expression of CD30 on CD4 Treg and non-Tregs. Groups of Tregs and non-Treg subpopulations were gated based on FOXP3 an CD25 expression (gates 1-4). As shown in FIG. 2, group 1 Tumor Tregs FOXP3^{hi} CD25^{hi} show high levels of CD30 expression compared to less activated Tregs or non-Treg CD4s in tumors (groups 1-3) or splenic CD4 T cells (groups 1-4). CD30 expression is restricted to the highly activated subset of Tregs in tumors in the A20 model.

### Example 17: CD30 Targeting ADC Significantly Slows Tumor Growth

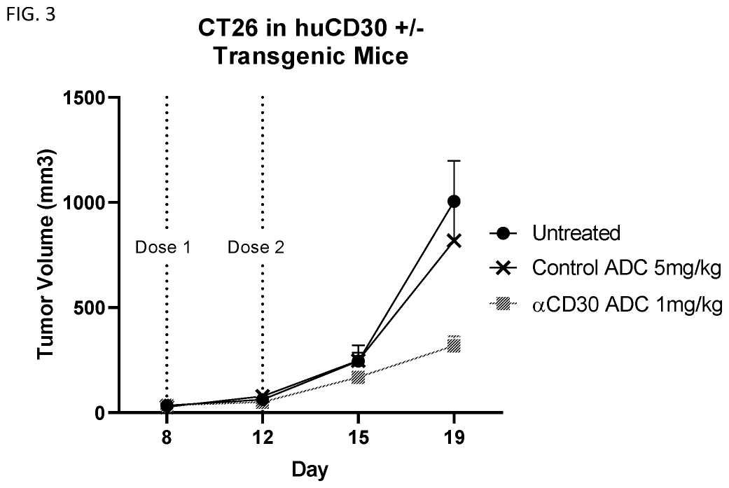

CT26 syngeneic colon carcinoma tumor cells were implanted s.c. into the left flank of huCD30 Tg+/− mice. Mice received two doses of a human CD30 targeting ADC (CD30-ADC) 1 mg/kg or a non-specific control ADC 5 mg/kg 4 days apart, with the first dose administered when tumor volumes averaged 50 mm3. Tumor volumes were monitored until the control group average exceeded 800 mm3. In this model implanted CT26 cells do not express human or mouse CD30, and huCD30 is only expected to be endogenously expressed by immune cells. As shown in FIG. 3, human CD30 targeting ADC significantly slows tumor growth of CT26 syngeneic colon carcinoma tumor cells.

### Example 18: CD30 Expression is Enriched on T Regulatory Cells in Dissociated NSCLC Tumors and is Upregulated Following Treatment with a PD1 Blocking Antibody

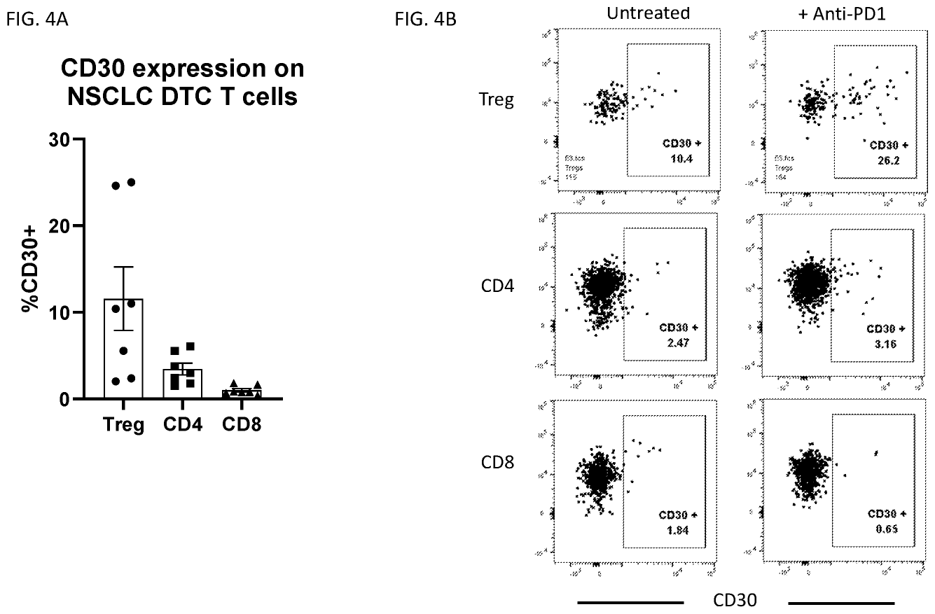

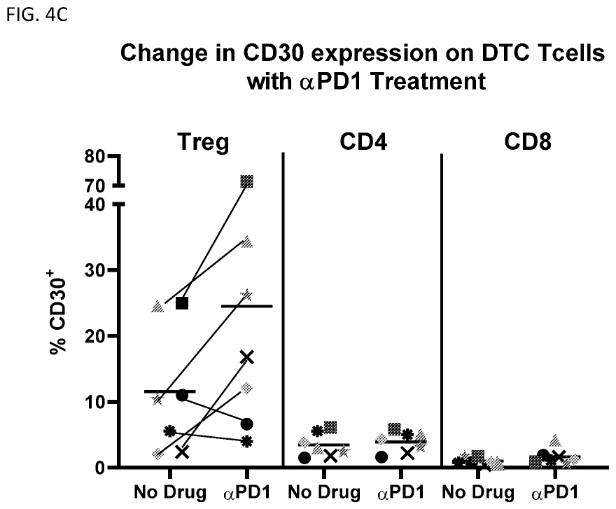

NSCLC tumors dissociated into single cell suspensions were purchased from Discovery Life Sciences. Dissociated tumor cells (DTCs) were resuspended in RPMI+10% FCS and plated in non-adherent 96 well round bottom plates to form DTC spheroids. Cells were left untreated or treated with 20 ug/ml of an anti-PD1 blocking antibody. After 4 days of culture at 37° C. DTC spheroids were stained for FACS analysis. DTCs were stained with viability dye and fluorescently labeled antibodies targeting CD3, CD2, CD4, CD8, CD30, FoxP3, and CD25 to discriminate CD4, CD8, and Treg (CD4+, FOXP3+, CD25+) T cells and the proportion of cells expressing CD30. In DTC cultures CD30 expression was enriched on Tregs relative to CD4 and CD8 T cells (FIG. 4A). DTC spheroids treated with an anti-PD-1 blocking antibody showed increases in the proportion of Tregs expressing CD30 in 5 of 7 DTC samples, while CD4 and CD8 T cells did not show increased CD30 expression (FIG. 4C)

1. A method of treating cancer in a subject, the method comprising administering to the subject an anti-PD-1 antibody or an antigen-binding fragment thereof and an anti-CD30 antibody-drug conjugate, wherein the cancer is a solid tumor selected from the group consisting of non-small cell lung cancer (NSCLC), melanoma, head and neck cancer, renal cell carcinoma, microsatellite instability-high or mismatch repair deficient cancer, bladder cancer, colon cancer, rectal cancer, gastric cancer, gastroesophageal junction carcinoma, esophageal cancer, cervical cancer, hepatocellular carcinoma, endometrial carcinoma, Merkel cell carcinoma, cutaneous squamous cell carcinoma, breast cancer, and tumor mutational burden-high cancer, and the medicament is to be administered to a subject in combination with an anti-PD-1 antibody or an antigen-binding fragment thereof, wherein the anti-PD-1 antibody or antigen-binding fragment thereof comprises a heavy chain variable region and a light chain variable region, wherein the heavy chain variable region comprises:
(i) a CDR-H1 comprising the amino acid sequence of SEQ ID NO:17;
(ii) a CDR-H2 comprising the amino acid sequence of SEQ ID NO:18; and
(iii) a CDR-H3 comprising the amino acid sequence of SEQ ID NO:19; and
wherein the light chain variable region comprises:
(i) a CDR-L1 comprising the amino acid sequence of SEQ ID NO:20;
(ii) a CDR-L2 comprising the amino acid sequence of SEQ ID NO:21; and
(iii) a CDR-L3 comprising the amino acid sequence of SEQ ID NO:22.

2. The method of claim 1, wherein the anti-PD-1 antibody or antigen-binding fragment thereof inhibits the activity of PD-1.

3. The method-of claim 1, wherein the anti-PD-1 antibody or antigen-binding fragment thereof comprises a heavy chain variable region comprising the amino acid sequence of SEQ ID NO:31 and a light chain variable region comprising the amino acid sequence of SEQ ID NO:32.

4. The method of claim 1, wherein the anti-PD-1 antibody or antigen-binding fragment thereof is pembrolizumab or a biosimilar thereof.

5. (canceled)

6. The method of claim 1, wherein the anti-CD30 antibody-drug conjugate comprises an anti-CD30 antibody or antigen-binding fragment thereof conjugated to a therapeutic agent.

7. The method of claim 6, wherein the anti-CD30 antibody or antigen-binding fragment thereof of the anti-CD30 antibody drug conjugate comprises a heavy chain variable region and a light chain variable region, wherein the heavy chain variable region comprises:
(i) a CDR-H1 comprising the amino acid sequence of SEQ ID NO:1;
(ii) a CDR-H2 comprising the amino acid sequence of SEQ ID NO:2; and
(iii) a CDR-H3 comprising the amino acid sequence of SEQ ID NO:3; and
wherein the light chain variable region comprises:
(i) a CDR-L1 comprising the amino acid sequence of SEQ ID NO:4;
(ii) a CDR-L2 comprising the amino acid sequence of SEQ ID NO:5; and
(iii) a CDR-L3 comprising the amino acid sequence of SEQ ID NO:6.

8. The method of claim 6, wherein the anti-CD30 antibody or antigen-binding fragment thereof of the anti-CD30 antibody drug conjugate comprises a heavy chain variable region comprising the amino acid sequence of SEQ ID NO:7 and a light chain variable region comprising the amino acid sequence of SEQ ID NO:8.

9. The method of claim 6, wherein the anti-CD30 antibody or antigen-binding fragment thereof of the anti-CD30 antibody drug conjugate is cAC10 or a biosimilar thereof.

10-15. (canceled)

16. The method of claim 1, wherein the anti-CD30 antibody-drug conjugate is brentuximab vedotin or a biosimilar thereof.

17. (canceled)

18. The method of claim 1, wherein the anti-PD-1 antibody or antigen-binding fragment thereof is administered at a flat dose ranging from about 50 mg to about 500 mg.

19-22. (canceled)

23. The method of claim 1, wherein the anti-PD-1 antibody or antigen-binding fragment thereof is administered once about every 1 week, once about every 2 weeks, once about every 3 weeks, once about every 4 weeks, once about every 5 weeks, or once about every 6 weeks.

24-28. (canceled)

29. The method of claim 1, wherein the anti-CD30 antibody-drug conjugate is administered at a dose of about 1.8 mg/kg of the subject's body weight.

30-31. (canceled)

32. The method of claim 29, wherein the anti-CD30 antibody-drug conjugate is administered once about every 3 weeks.

33. (canceled)

34. The method of claim 1, wherein the subject has not been previously treated for the cancer.

35. The method of claim 1, wherein the subject has been previously treated for the cancer and the subject failed treatment, did not respond to treatment, progressed on treatment, or relapsed after first-line treatment.

36. The method of claim 35, wherein the subject failed treatment, did not respond to treatment, progressed on treatment, or relapsed after treatment with an anti-PD-1 monoclonal antibody.

37. (canceled)

38. The method of claim 35, wherein the subject was on a PD-1 checkpoint inhibitor therapy as the last previous line of therapy within 90 days of being administered the combination of the anti-PD-1 antibody or an antigen-binding fragment thereof and the anti-CD30 antibody-drug conjugate.

39. The method of claim 35, wherein the subject was previously treated for the cancer with surgery.

40. (canceled)

41. The method of claim 1, wherein the cancer is metastatic.

42. The method of claim 1, wherein the cancer is NSCLC.

43-47. (canceled)

48. The method of claim 42, wherein the subject progressed after having developed a prior objective response of complete response or partial response (CR/PR) for at least 3 months or stable disease for at least 6 months.

49. The method of claim 42, wherein the subject does not have a known targetable EGFR, ALK, ROS1, or BRAF mutation.

50. The method of claim 1, wherein the cancer is melanoma.

51-55. (canceled)

56. The method of claim 50, wherein the subject does not have a targetable gene mutation.

57. The method of claim 50, wherein the subject is a BRAF-V660E/V600K subject who failed targeted therapy.

58-79. (canceled)

80. The method of claim 1, wherein the subject has not been previously treated with the anti-CD30 antibody-drug conjugate.

81. The method of claim 1, wherein the subject does not have known active central nervous system metastases and/or carcinomatous meningitis.

82-85. (canceled)

86. The method of claim 1, wherein the anti-PD-1 antibody or antigen-binding fragment thereof and the anti-CD30 antibody-drug conjugate are administered sequentially.

87. The method of claim 1, wherein the anti-PD-1 antibody or antigen-binding fragment thereof and the anti-CD30 antibody-drug conjugate are administered simultaneously.

88. The method of claim 1, wherein at least about 0.1%, at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, or at least about 80% of cancer cells from the subject express PD-L1.

89-93. (canceled)

94. The method of claim 1, wherein less than about 0.1%, less than about 0.5%, less than about 1%, less than about 2%, less than about 3%, less than about 4%, or less than about 5% of cancer cells from the subject express CD30.

95. The method of claim 1, wherein the cancer cells from the subject do not express CD30.

96-120. (canceled)


SEQUENCE LISTING (34 sequences)

SEQ ID NO: 1 - protein, 5 aa, synthetic construct
DYYIT

SEQ ID NO: 2 - protein, 17 aa, synthetic construct
WIYPGSGNTKYNEKFKG

SEQ ID NO: 3 - protein, 8 aa, synthetic construct
YGNYWFAY

SEQ ID NO: 4 - protein, 15 aa, synthetic construct
KASQSVDFDGDSYMN

SEQ ID NO: 5 - protein, 7 aa, synthetic construct
AASNLES

SEQ ID NO: 6 - protein, 9 aa, synthetic construct
QQSNEDPWT

SEQ ID NO: 7 - protein, 117 aa, synthetic construct
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, synthetic construct
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 30 aa, synthetic construct
QIQLQQSGPEVVKPGASVKISCKASGYTFT

SEQ ID NO: 10 - protein, 14 aa, synthetic construct
WVKQKPGQGLEWIG

SEQ ID NO: 11 - protein, 32 aa, synthetic construct
KATLTVDTSSSTAFMQLSSLTSEDTAVYFCAN

SEQ ID NO: 12 - protein, 11 aa, synthetic construct
WGQGTQVTVSA

SEQ ID NO: 13 - protein, 23 aa, synthetic construct
DIVLTQSPASLAVSLGQRATISC

SEQ ID NO: 14 - protein, 15 aa, synthetic construct
WYQQKPGQPPKVLIY

SEQ ID NO: 15 - protein, 32 aa, synthetic construct
GIPARFSGSGSGTDFTLNIHPVEEEDAATYYC

SEQ ID NO: 16 - protein, 10 aa, synthetic construct
FGGGTKLEIK

SEQ ID NO: 17 - protein, 5 aa, synthetic construct
NYYMY

SEQ ID NO: 18 - protein, 17 aa, synthetic construct
GINPSNGGTNFNEKFKN

SEQ ID NO: 19 - protein, 11 aa, synthetic construct
RDYRFDMGFDY

SEQ ID NO: 20 - protein, 15 aa, synthetic construct
RASKGVSTSGYSYLH

SEQ ID NO: 21 - protein, 9 aa, synthetic construct
QHSRDLPLT

SEQ ID NO: 22 - protein, 7 aa, synthetic construct
LASYLES

SEQ ID NO: 23 - protein, 30 aa, synthetic construct
QVQLVQSGVEVKKPGASVKVSCKASGYTFT

SEQ ID NO: 24 - protein, 14 aa, synthetic construct
WVRQAPGQGLEWMG

SEQ ID NO: 25 - protein, 32 aa, synthetic construct
RVTLTTDSSTTTAYMELKSLQFDDTAVYYCAR

SEQ ID NO: 26 - protein, 11 aa, synthetic construct
WGQGTTVTVSS

SEQ ID NO: 27 - protein, 23 aa, synthetic construct
EIVLTQSPATLSLSPGERATLSC

SEQ ID NO: 28 - protein, 15 aa, synthetic construct
WYQQKPGQAPRLLIY

SEQ ID NO: 29 - protein, 32 aa, synthetic construct
GVPARFSGSGSGTDFTLTISSLEPEDFAVYYC

SEQ ID NO: 30 - protein, 10 aa, synthetic construct
FGGGTKVEIK

SEQ ID NO: 31 - protein, 120 aa, synthetic construct [sequence omitted]

SEQ ID NO: 32 - protein, 111 aa, synthetic construct [sequence omitted]

SEQ ID NO: 33 - protein, 446 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC